SEQUENCE LISTING (21 sequences)

SEQ ID NO: 1 - protein, 379 aa, synthetic construct
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

SEQ ID NO: 2 - DNA, 1140 bp, synthetic construct
  source 1..1140: mol_type = other DNA
atgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctga

SEQ ID NO: 3 - DNA, 2292 bp, synthetic construct
  source 1..2292: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 2959 bp, synthetic construct
  source 1..2959: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctga

SEQ ID NO: 5 - DNA, 3543 bp, synthetic construct
  source 1..3543: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - DNA, 4979 bp, synthetic construct
  source 1..4979: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 6695 bp, synthetic construct
  source 1..6695: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggagatctacaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 6230 bp, synthetic construct
  source 1..6230: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 670 bp, synthetic construct
  source 1..670: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgg

SEQ ID NO: 10 - DNA, 1182 bp, synthetic construct
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 11 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 106 bp, synthetic construct
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 15 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 16 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 17 - DNA, 1009 bp, synthetic construct
  source 1..1009: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 18 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 1965 bp, synthetic construct
  source 1..1965: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatctta

SEQ ID NO: 20 - DNA, 3594 bp, synthetic construct
  source 1..3594: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 3194 bp, synthetic construct
  source 1..3194: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

(19) United States
(12) Patent Application Publication
(10) Pub. No.: US 2024/0390516 A1
(43) Pub. Date: Nov. 28, 2024

(54) ADENO-ASSOCIATED VECTORS AND VIRIONS TO TREAT GALACTOSEMIA AND METHODS OF USE AND MANUFACTURE

(71) Applicants: Jaguar Gene Therapy, LLC, Lake Forest, IL (US); Emory University, Atlanta, GA (US); University of Utah Research Foundation, Salt Lake City, UT (US)

(72) Inventors: Michael Hughes, London (GB); Kevin Foust, Ponway, CA (US); Sandeep Dhall, Columbia, MD (US); Judith L. Fridovich-Keil, Atlanta, GA (US); Shauna A. Rasmussen, Atlanta, GA (US); Jennifer M.I. Daenzer, Altanta, GA (US); Kent Lai, Salt Lake City, UT (US)

(21) Appl. No.: 18/688,724

(22) PCT Filed: Sep. 1, 2022
(86) PCT No.: PCT/US2022/075869

§ 371 Date: Mar. 1, 2024

Related U.S. Application Data
(60) Provisional application No. 63/239,650, filed on Sep. 1, 2021.
(60) Provisional application No. 63/331,868, filed on Apr. 17, 2022.
(60) Provisional application No. 63/331,870, filed on Apr. 17, 2022.
(60) Provisional application No. 63/342,128, filed on May 15, 2022.

(51) Int. Cl. A61K 48/00; A61K 38/45; A61P 3/00

(52) U.S. Cl. CPC A61K 48/0033; A61K 38/45; A61K 48/0066; A61K 48/0075; A61K 48/0083; A61P 3/00; C12N 2750/14143; C12N 2750/14171

(57) ABSTRACT

Provided are recombinant adeno-associated virus (r.AAV) vectors comprising a transgene to express galactose-1-phosphate uridylyl transferase (GALT); virions comprising said vectors (r.AAV virions); methods of their production; methods of their use, including methods for treating galactosemia, GALT-deficiency, symptoms therefrom: and kits. These recombinant adeno-associated virus (rAAV) vectors comprise a method of treating galactosemia in a subject in need.

### FIELD OF THE INVENTION

Provided are viral constructs, particles and compositions for use in the treatment of galactose-1-phosphate uridylyltransferase (GALT) deficiency.

### BACKGROUND

Galactosemia results from the inability to metabolize galactose, and type 1 galactosemia is specifically caused by pathogenic variants in the GALT gene encoding the enzyme galactose-1-phosphate uridylyltransferase (GALT). Accordingly, type 1 galactosemia can also be known as galactose-1-phosphate uridylyltransferase deficiency. GALT performs the second step of the Leloir pathway of galactose metabolism. In that step, GALT interconverts uridine diphosphate-glucose and galactose-1-phosphate into glucose-1-phosphate and uridine diphosphate-galactose in a reversible manner.

Type 1 galactosemia can be categorized into three types of disease. The first is classic galactosemia, which occurs when the individual has pathogenic variants in both of their GALT alleles resulting in lost expression of GALT or a complete or near complete (˜1% residual activity) loss of GALT catalytic activity. Classic galactosemia is an autosomal recessive disorder. The second category of type 1 galactosemia is described as “clinical variant” galactosemia in which patients maintain between 1% to 10% of normal enzyme activity levels in erythrocytes or other tissues. The third type is a biochemical variant galactosemia, called Duarte galactosemia, which occurs when an individual is a compound heterozygote for GALT alleles that combined express around 25% of the level of GALT activity seen in an individual having two normal GALT alleles. An example of a mutation causing classic galactosemia is a nucleotide substitution from A to G in exon 6 of the GALT gene, which substitutes an arginine in place of glutamine at position 188 in the protein.

Type 1 galactosemia disease process starts in utero (Holton 1995). Patients with Type 1 galactosemia with GALT enzyme activity <10% of normal levels are at risk of neonatal death if dietary galactose (primarily from lactose) is not removed. Signs of Type 1 galactosemia in neonates include difficulty feeding, failure to thrive, jaundice, and liver damage, sometimes leading to liver failure (Berry 2021; Rubio-Gozalbo et al 2019). The GalNet registry, which collected data from 509 patients in 15 countries from December 2014 to July 2018, showed that 26% of patients experienced cataracts in the neonatal period (Rubio-Gozalbo et al 2019). The timing, amount, and duration of exposure to galactose affect the extent of damage in infants with galactosemia, with maximum damage incurred by early exposure to larger amounts for a longer duration. Morbidity during the first 10 days of life was reported to reach up to 75% of neonates with classic galactosemia prior to the onset of newborn screening practices and dietary restriction, with most deaths attributed to sepsis by Escherichia coli. The damage incurred by the galactosemia-sepsis complex extends beyond hepatocellular necrosis, hemosiderosis, fatty regeneration, and acinar formation of hepatocytes to pancreatic islet hyperplasia, renal cortical necrosis, periventricular leukomalacia, and meningitis (Kotb et al 2019). Infants quickly switched from breast milk or standard formula to soy-based or other low-galactose baby formulas generally recover quickly, without the most severe acute complications such as sepsis, liver failure, and neonatal death (Berry 2021, Welling et al 2017).

There is evidence that the causes of long-term complications of patients with classic galactosemia begin during gestation and are present in very early childhood. Despite dietary intervention, patients still experience significant long-term complications of the disease due to endogenous galactose production of 1-2 grams per day (Bosch 2006; Berry 2021). Clinical evaluations of patients with Type 1 galactosemia show early onset of symptoms. These patients are more likely to have neurological deficits, motor, cognitive and speech/language pathology as compared to age matched controls, which is evident in children as young as 48 months of age (Ozgun et al 2019). Data from the GalNet registry also demonstrate that motor symptoms (tremor) are present in 23.8% of patients between the first year and pre-school age. These data support the need for an early intervention strategy to mitigate or significantly reduce long-term disease outcomes (Rubio-Gozalbo et al 2019) which is made feasible by the fact that there is newborn screening for classic galactosemia in many developed countries.

The GalNet registry also provides information on the overall impact of these long-term complications based on the 509 patients assessed (Rubio-Gozalbo et al 2019; Berry 2021). Neurological, cognitive, and behavioral complications were common in patients in the GalNet registry. Brain impairments, which is inclusive of multiple cognitive and mental complications, were reported in 85.0% of patients with global developmental delays reported in 52.2% of patients, and language and speech disorders reported in 66.4% of patients. Language and speech disorders were more commonly reported in young male patients (p=0.034) (Rubio-Gozalbo et al 2019). In total, 52.0% of patients had neurological complications, such as tremor (31.0%), general motor abnormality (27.0%), ataxia (12.2%), seizures (8.1%), and dystonia (7.5%). General motor abnormality was reported most frequently at preschool age, but ataxia, seizures, and dystonia were reported across the ages. In the GalNet registry, 44.4% of patients had mental (psychiatric) and behavioral problems, including anxiety disorder (22.3%), depression (12.5%), attention-deficit/hyperactivity disorder (7.3%), and autism spectrum disorder (6.0%) (Rubio-Gozalbo et al 2019). Growth is also severely delayed during childhood and early adolescence with a significant number of patients beneath the tenth percentile for height and beneath the fiftieth percentile for weight (Waggoner et al 1990).

One of the most common and debilitating complications of classic galactosemia patients is speech and sound disorders (Hughes et al 2009; Rubio-Gozalbo et al 2019; Waggoner et al 1990), with 24-63% of children with Type 1 galactosemia reporting a more severe childhood apraxia of speech (CAS) (Shriberg et al 2011a; Waggoner et al 1990; Waisbren et al 2012; Webb et al 2003); children with speech disorders secondary to galactosemia are conservatively at a six- to eight-fold greater risk and estimated to be as high as 180 times the risk for CAS as compared to children with speech disorders of unknown origin (Potter et al 2008; Shriberg et al 2011b). CAS is a motor speech disorder caused by disruptions in higher-level motor commands, neuromuscular system impairments, or both and is characterized by inconsistent consonant and vowel errors, difficult transitioning between articulatory movements, and inappropriate prosody during speech (Duffy 2005; American Speech-Language-Hearing Association 2007). CAS may result from disruption in higher-level motor commands primarily in the left hemisphere Broca's area, supplementary motor area, or insula and may involve the cerebellum and basal ganglia leading to difficulty in planning and programming the sequence of speech movements (Potter 2011). CAS is not a disorder that can be outgrown; rather children with CAS will not make progress without treatment. Approximately 50-78% of children with classic galactosemia were reported to have a developmental language disorder (DLD) (Potter et al 2008; Rubio-Gozalbo et al 2019; Waggoner et al 1990). Children with DLD have difficulty using and/or understanding language and have language abilities that fall behind those of other children their age, even with similar IQ ranges. Children with DLD may have difficulty socializing with their classmates, talking about how they feel, and learning in school and although DLD is usually first discovered and treated in childhood, it usually does not go away as a child grows up (Norbury et al 2016). Many of the neurological complications of classic galactosemia are multifactorial with patients often exhibiting multiple complications coincidentally (i.e., cognitive disability and speech delay). Improved therapies are required for these patients.

### SUMMARY OF INVENTION

Provided are nucleic acid vector constructs for production of recombinant adeno-associated virus (AAV) virions encoding GALT. The vector constructs are used to generate recombinant AAV virions or particles which are administered for rAAV gene therapy in a subject suffering from galactosemia to deliver to cells of the subject a nucleic acid encoding GALT, thereby providing increased GALT activity to the subject to treat and ameliorate disease.

Provided are AAV vectors comprising an expression cassette which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences. The hGALT may have the amino acid sequence of SEQ ID NO:1 and may be encoded by a nucleic comprising a nucleotide sequence having at least 85% identity to the nucleotide sequence of SEQ ID NO.: 2 or a sequence reverse complement thereof, which encodes an hGALT, and, may comprise the nucleotide sequence of SEQ ID NO: 2. The vector may also contain a WPRE element, which may be located between the hGALT coding sequence and the polyA signal sequence. The ITR sequences may be wild type sequences, such as AAV2 ITRs or may include a modified ITR sequence that results in a double-stranded “self complementary” AAV vector.

Particular expression cassettes (i.e., recombinant AAV genomes having the transgene, regulatory sequences and ITR sequences) are provided and have nucleotide sequences of SEQ ID NO.: 3 (pscAAV-CAG-hGALT), SEQ ID NO.: 4 (pAAV2ITR-CAG-hGALT), or SEQ ID NO.: 5 (pAAV2ITR-EF1a-hGALT) (or a sequence reverse complementary thereto), or may be at least 85% identical to the nucleotide sequence of SEQ ID NO.: 3 (pscAAV-CAG-hGALT), SEQ ID NO.: 4 (pAAV2ITR-CAG-hGALT), or SEQ ID NO.: 5 (pAAV2ITR-EF1a-hGALT), and encodes and expresses a human GALT.

Particular expression cassettes (not including any flanking ITRs sequences) are provided and have nucleotide sequences of SEQ ID NO.: 19 (pscAAV CAG-hGALT), SEQ ID NO.: 20 (pAAV2ITR-CAG-hGALT), or SEQ ID NO.: 21 (pAAV2ITR EF1a-hGALT) (or a sequence reverse complementary thereto), or may be at least 85% identical to the nucleotide sequence of SEQ ID NO.: 19 (pscAAV-CAG-hGALT), SEQ ID NO.: 20 (pAAV2ITR-CAG-hGALT), or SEQ ID NO.: 21 (pAAV2ITR-EF1a-hGALT), and encodes and expresses a human GALT.

Also provided are plasmids comprising these expression cassettes, including the plasmid pAAV2ITR-CAG-hGALT-KanR, depicted in FIG. 1, the nucleotide sequence of which is provided in FIG. 2 (also the nucleotide sequence of SEQ ID NO: 7 and the reverse complement thereof); the plasmid pAAV2ITR-EF1a-hGALT-KanR, depicted in FIG. 3, the nucleotide sequence of which is provided in FIG. 4 (also nucleotide sequence of SEQ ID NO: 8) and pscAAV-CAG-hGALT-KanR, depicted in FIG. 5, the nucleotide sequence of which is provided in FIG. 6 (also the nucleotide sequence of SEQ ID NO: 6).

Also provided are recombinant AAV virions comprising a recombinant AAV genome (an expression cassette) described herein encoding hGALT (for example, the expression cassettes of SEQ ID NO: 3, 4 or 5) and an AAV capsid. The AAV capsid may be an AAV9 capsid (amino acid sequence SEQ ID NO: 18).

Also provided are methods of treating galactosemia or in increasing galactose metabolism in a subject in need thereof, particularly, a human subject. Also included are methods of reducing a disease condition in a subject suffering from galactosemia by administering rAAV virions described herein, wherein the disease condition comprises jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency. Provided are pharmaceutical compositions including for use in treating galactosemia or increasing galactose metabolism in a subject in need thereof and methods of administration, including, but not limited to, intravenous administration or intrathecal administration.

Also provided are host cells for and methods of producing the recombinant AAV virions as described herein.

Also provided are methods of reducing galactose levels in a subject in need thereof, methods of reducing galactitol levels in a subject in need thereof, methods of reducing galactose-1-phosphate (Gal-1P) levels in a subject in need thereof, methods of inhibiting cataract formation or promoting cataract resorption in a subject in need thereof, methods of limiting the severity of cataract formation in a subject in need thereof and methods of preventing inhibited growth in a subject in need thereof.

Also provided are methods of increasing GALT enzyme activity in a subject in need thereof including in liver, brain and skeletal muscles.

### EMBODIMENTS

1. A recombinant adeno-associated virus (AAV) vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences.

2. The recombinant AAV vector of embodiment 1, the hGALT comprising the amino acid sequence of SEQ ID NO.: 1.

3. The recombinant AAV vector of embodiment 1 or embodiment 2, the nucleic acid that encodes GALT comprising a nucleic acid having at least 85% identity to the nucleotide sequence of SEQ ID NO.: 2 or a sequence reverse complementary thereto.

4. The recombinant AAV vector of any one of embodiments 1-3, the nucleic acid that encodes GALT comprising or consisting of the nucleotide sequence of SEQ ID NO.: 2 or a sequence reverse complementary thereto.

5. The recombinant AAV vector of any one of embodiments 1-4 wherein the promoter has a nucleotide sequence of SEQ ID NO: 9 or SEQ ID NO: 10, or the reverse complement thereof, and the polyA signal sequence has a nucleotide sequence of SEQ ID NO: 15 or SEQ ID NO: 16, or the reverse complement thereof.

6. The recombinant AAV vector of any one of embodiments 1-5, which comprises a WPRE element.

7. The recombinant AAV vector of any one of embodiments 1-6, wherein the ITRs comprise a 5′ AAV2 ITR having a nucleotide sequence of SEQ ID NO: 11 and a 3′ AAV2 ITR having a nucleotide sequence of SEQ ID NO: 12, or the reverse complement thereof.

8. The recombinant AAV vector of any one of embodiments 1-6 wherein the ITRs comprise a 5′ ITR having a nucleotide sequence of SEQ ID NO: 11 and a modified self-complementary 3′ ITR having a nucleotide sequence of SEQ ID NO: 13, or reverse complement thereof.

9. The recombinant AAV vector of any one of embodiments 1-8 comprising an expression cassette comprising a nucleic acid that has at least 85% identity to the nucleotide sequence of SEQ ID NO.: 3, SEQ ID NO.: 4, or SEQ ID NO.: 5, or a sequence reverse complementary thereto, and encodes a human GALT.

10. The recombinant AAV vector of any one of embodiments 1-8 comprising an expression cassette comprising a nucleic acid that has at least 85% identity to the nucleotide sequence of SEQ ID NO.: 19, SEQ ID NO.: 20, or SEQ ID NO.: 21, or a sequence reverse complementary thereto, and encodes a human GALT.

11. The recombinant AAV vector of any one of embodiments 1-9 comprising an expression cassette comprising a nucleic acid having a nucleotide sequence of SEQ ID NO.: 3, SEQ ID NO.: 4, or SEQ ID NO.: 5, or a sequence reverse complementary thereto.

12. The recombinant AAV vector of any one of embodiments 1-8 or embodiment 10 comprising an expression cassette comprising a nucleic acid having a nucleotide sequence of SEQ ID NO.: 19, SEQ ID NO.: 20, or SEQ ID NO.: 21, or a sequence reverse complementary thereto.

13. A recombinant self-complementary AAV (scAAV) vector comprising the recombinant AAV vector of any one of embodiments 1-12 comprising an scAAV ITR.

14. A recombinant AAV virion comprising: 1) an AAV capsid; and 2) the recombinant AAV vector of any one of embodiments 1-13; and the AAV capsid protein encapsulating the recombinant AAV vector.

15. The AAV virion of embodiment 14, wherein the AAV capsid has an amino acid sequence at least 85% identical to SEQ ID NO: 18 (AAV9).

16. The AAV virion of embodiment 15, wherein the AAV capsid has an amino acid sequence of SEQ ID NO: 18.

17. A method for treating galactosemia in a subject in need thereof, the method comprising administering to the subject a therapeutically effective amount of the AAV virion of any one of embodiments 14-16.

18. A method of increasing galactose metabolism in a subject in need thereof, the method comprising administering to the subject a therapeutically effective amount of the AAV virion of any one of embodiments 14-16.

19. A method of reducing a disease condition in a subject suffering from galactosemia, the method comprising administering to the subject a therapeutically effective amount of the AAV virion of any one of embodiments 14-16, the disease condition comprising jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency.

20. The method of any one of embodiments 17-19, said administering comprising intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation.

21. The method of any one of embodiments 17-19, the administering comprising intravenous administration, or intrathecal administration.

22. An AAV vector plasmid comprising 1) an origin of replication and 2) the recombinant AAV vector of any one of embodiments 1-13.

23. The AAV vector plasmid of embodiment 22, comprising a nucleic acid having at least 85% identity to the nucleotide sequence of SEQ ID NO.: 6, SEQ ID NO.: 7 or SEQ ID NO.: 8, which encodes a human GALT.

24. The AAV vector plasmid of embodiment 22, comprising a nucleic acid having at least 85% identity to the nucleotide sequence of SEQ ID NO.: 19, SEQ ID NO.: 20 or SEQ ID NO.: 21, which encodes a human GALT.

25. The AAV vector plasmid of embodiment 22 or 23 comprising the nucleotide sequence of SEQ ID NO.: 6, SEQ ID NO.: 7, or SEQ ID NO.: 8.

26. The AAV vector plasmid of embodiment 22 or 24 comprising the nucleotide sequence of SEQ ID NO.: 19, SEQ ID NO.: 20, or SEQ ID NO.: 21.

27. A cell comprising an AAV vector plasmid of any one of embodiments 22-26 and a second plasmid comprising nucleotide sequences encoding rep and cap; the cap encoding a VP1, a VP2, and a VP3; the rep encoding rep78, rep68, rep 52, and rep 40.

28. The cell of embodiment 27, the cap being AAV9 cap.

29. A method of producing an AAV virion, the method comprising culturing a host cell comprising the AAV vector plasmid of any one of embodiments 22-26, a second plasmid encoding the cap and rep; the cap encoding the VP1, the VP2, and the VP3; the rep encoding rep78, rep68, rep 52, and rep 40; and any additional adenoviral helper functions, under conditions sufficient to produce the AAV virion; and isolating the AAV virion produced by the host cell.

30. A method of reducing galactose levels in a subject in need thereof, the method comprising administering to the subject a therapeutically effective amount of the AAV virion of any one of embodiments 14-16.

31. A method of reducing galactitol levels in a subject in need thereof, the method comprising administering to the subject a therapeutically effective amount of the AAV virion of any one of embodiments 14-16.

32. A method of reducing galactose-1-phosphate (Gal-1P) in a subject in need thereof, the method comprising administering to the subject a therapeutically effective amount of the AAV virion of any one of embodiments 14-16.

33. The method of any one of embodiments 30-32, wherein galactose levels, galactitol levels and/or Gal-1P levels are reduced in liver, muscle, brain, eye, ovary, red blood cells and/or blood plasma.

34. The method of embodiment 33, wherein galactose and galactitol levels are reduced in blood plasma.

35. The method of embodiment 33, wherein galactose, galactitol and Gal-1P levels are reduced in red blood cells.

36. A method of increasing GALT protein expression in a subject in need thereof, the method comprising administering to the subject a therapeutically effective amount of the AAV virion of any one of embodiments 14-16.

37. A method of increasing GALT enzyme activity in a subject in need thereof, the method comprising administering to the subject a therapeutically effective amount of the AAV virion of any one of embodiments 14-16.

38. The method of any one of embodiments 36-37, wherein GALT protein expression and/or GALT enzyme activity is increased in liver, muscle, brain, eye, red blood cells and/or blood plasma.

39. The method of any one of embodiments 36-37, wherein GALT protein expression and/or GALT enzyme activity is increased in brain.

40. The method of embodiment 39, wherein GALT protein expression and/or GALT enzyme activity is increased in cortex and/or cerebral tissues.

41. The method of embodiment 40, wherein GALT protein expression and/or GALT enzyme activity is increased in neural and/or glial cells.

42. The method of embodiment 41, wherein GALT protein expression and/or GALT enzyme activity is increased in Purkinje neurons.

43. A method of improving motor and/or neuromuscular coordination comprising administering to the subject a therapeutically effective amount of the AAV virion of any one of embodiments 14-16.

44. A method of improving motor strength comprising administering to the subject a therapeutically effective amount of the AAV virion of any one of embodiments 14-16.

45. A method of improving spatial learning comprising administering to the subject a therapeutically effective amount of the AAV virion of any one of embodiments 14-16.

46. A method of reducing and/or rescuing ovarian failure comprising administering to the subject a therapeutically effective amount of the AAV virion of any one of embodiments 14-16.

47. A method of regulating follicle-stimulating hormone, luteinizing hormone and/or anti-Müllerian hormone comprising administering to the subject a therapeutically effective amount of the AAV virion of any one of embodiments 14-16.

48. A method of inhibiting cataract formation or promoting cataract resorption in a subject in need thereof, the method comprising administering to the subject a therapeutically effective amount of the AAV virion of any one of embodiments 14-16.

49. A method of limiting the severity of cataract formation in a subject in need thereof, the method comprising administering to the subject a therapeutically effective amount of the AAV virion of any one of embodiments 14-16.

50. The method of embodiment 48 or embodiment 49, wherein said therapeutically effective amount of the AAV virion is administered prior to the development of cataracts.

51. A method of reducing a pre-pubertal growth delay in a subject in need thereof, the method comprising administering to the subject a therapeutically effective amount of the AAV virion of any one of embodiments 14-16.

52. A method of improving weight gain in a subject in need thereof, the method comprising administering to the subject a therapeutically effective amount of the AAV virion of any one of embodiments 14-16.

53. A method of reducing the severity of a visceral neuromuscular deficit in a subject in need thereof, the method comprising administering to the subject a therapeutically effective amount of the AAV virion of any one of embodiments 14-16.

54. A method of reducing the severity of neurological and/or socioemotional deficits in a subject in need thereof, the method comprising administering to the subject a therapeutically effective amount of the AAV virion of any one of embodiments 14-16.

55. The method of embodiment 54, wherein the neurological and/or socioemotional deficit is locomotor function.

56. The method of embodiment 55, wherein the locomotor function is tremor and/or ataxia.

57. The method of embodiment 54, wherein the neurological and/or socioemotional deficit is anxiety.

58. The method of embodiment 54, wherein the neurological and/or socioemotional deficit is depression.

59. The method of any one of embodiments 17-21 or 30-58, wherein the AAV virion is administered prior to onset of puberty.

60. The method of embodiment 59, wherein the AAV virion is administered to a young pediatric subject or a pediatric subject.

61. The method of any one of embodiments 17-21 or 30-60, further comprising the step of monitoring levels of galactose, GAL-1P and/or galactitol.

### BRIEF DESCRIPTION OF THE DRAWINGS

FIG. 1 depicts a map of the plasmid pAAV2ITR-CAG-hGALT-KanR, which comprises two AAV 2 inverted-terminal repeats (ITR) (SEQ ID NO.:11 and 12) flanking an expressing region comprising a cytomegalovirus (CMV) early enhancer/chicken β-actin/rabbit β-globin splice acceptor (CAG) promoter (SEQ ID NO.: 9), a nucleic acid encoding human GALT (hGALT) (SEQ ID NO.:2), a woodchuck hepatitis virus post-transcriptional regulatory element (WPRE) (SEQ ID NO.:14), and a bovine growth hormone poly adenylation (bGH poly(A)) signal sequence (SEQ ID NO.:15). The plasmid further comprises an ampicillin resistance gene (AmpR) promoter controlling expression of a kanamycin resistance gene sequence (KanR), a CMV origin of replication (ori), and catabolite activator protein (CAP) binding site, a lactose (lac) promoter controlling a lac operator, and an M13 reverse primer region.

FIG. 2 provides the double stranded nucleotide sequence of the pAAV2ITR-CAG-hGALT-KanR plasmid (SEQ ID NOs.: 7), which includes the nucleotide sequence encoding the hGALT (SEQ ID NO.: 2) and the amino acid sequence of the hGALT (SEQ ID NO.: 1).

FIG. 3 depicts a map of the plasmid pAAV2ITR-EF1-hGALT-KanR, which comprises two AAV 2 ITRs flanking an expressing region comprising a elongation factor-1α (EF-1α) promoter (SEQ ID NO.:10), a nucleic acid encoding hGALT, WPRE, and bGH poly(A) signal sequence. The plasmid further comprises an AmpR promoter controlling expression of a KanR gene sequence, a CMV origin of replication (ori), and CAP binding site, a lac promoter controlling a lac operator, and an M13 reverse primer region.

FIG. 4 provides the double stranded nucleotide sequence of pAAV2ITR-EF1-hGALT-KanR plasmid (SEQ ID NOs.: 8), which includes the nucleotide sequence encoding the hGALT (SEQ ID NO.: 2) and the amino acid sequence of the hGALT (SEQ ID NO.: 1).

FIG. 5 depicts a map of the plasmid pscAAV-CAG-hGALT-KanR, which comprises one AAV 2 ITR upstream of an expressing region comprising the CAG promoter, a nucleic acid sequence encoding hGALT, WPRE, a simian virus 40 (SV40) poly(A) signal sequence (SEQ ID NO.:16), and a self-complementary AAV (scAAV) ITR (SEQ ID NO.: 13). The plasmid further comprises an AmpR promoter controlling expression of a KanR gene sequence, a CMV origin of replication (ori), and CAP binding site, a lac promoter controlling a lac operator, and an M13 reverse primer region.

FIG. 6 provides the double stranded nucleotide sequence of pscAAV-CAG-hGALT-KanR plasmid (SEQ ID NOs.: 6), which includes the nucleotide sequence encoding the hGALT (SEQ ID NO.: 2) and the amino acid sequence of the hGALT (SEQ ID NO.: 1).

FIGS. 7A-7G show the concentration of galactosemia biomarker Gal-1P in brain tissue (A,B), skeletal muscle tissue (C,D) and RBC's (E,F) from wild type (WT/WT) and GALT null (GG/GG) mice at 4 weeks and 12 weeks and liver tissue (G) at 12 weeks post-treatment with high dose AAV9-CAG-hGalt (1.15×10¹⁴ vg/kg), low dose AAV9-CAG-hGalt (3.74×10¹³ vg/kg) or AAV9-CAG-hGalt formulation buffer vehicle control. AAV9-CAG-hGalt significantly reduces Gal-1P levels in the brain, skeletal muscle, RBCs, and liver of GALT null mice.

FIGS. 8A-8I show the concentration of galactosemia biomarker galactose in brain tissue (A,B), skeletal muscle tissue (C,D), RBC's (E,F) and plasma (H,I) from wild type (WT/WT) and GALT null (GG/GG) mice at 4 weeks and 12 weeks and liver tissue (G) at 12 weeks post-treatment with high dose AAV9-CAG-hGalt (1.15×10¹⁴ vg/kg), low dose AAV9-CAG-hGalt (3.74×10¹³ vg/kg) or AAV9-CAG-hGalt formulation buffer vehicle control. AAV9-CAG-hGalt significantly reduces galactose levels in the brain, skeletal muscle, RBCs, plasma and liver of GALT null mice.

FIGS. 9A-9I show the concentration of galactosemia biomarker galactitol in brain tissue (A,B), skeletal muscle tissue (C,D), RBC's (E,F), and plasma (H,I) from wild type (WT/WT) and GALT null (GG/GG) mice at 4 weeks and 12 weeks and liver tissue (G) at 12 weeks post-treatment with high dose AAV9-CAG-hGalt (1.15×10¹⁴ vg/kg), low dose AAV9-CAG-hGalt (3.74×10¹³ vg/kg) or AAV9-CAG-hGalt formulation buffer vehicle control. AAV9-CAG-hGalt significantly reduces galactitol levels in the brain, skeletal muscle, RBCs, plasma and liver of GALT null mice.

FIGS. 10A-10C show the evaluation of GALT protein expression via immunohistochemistry liver (A), muscle (B) and brain (C) from wild type (WT/WT) and GALT null (GG/GG) mice at 4 weeks and 12 weeks post-treatment with high dose AAV9-CAG-hGalt (1.15×10¹⁴ vg/kg), low dose AAV9-CAG-hGalt (3.74×10¹³ vg/kg) or AAV9-CAG-hGalt formulation buffer vehicle control. AAV9-CAG-hGalt induced GALT enzyme expression in GALT null mouse pups.

FIGS. 11A-11C show the Experimental Design for behavioral studies (FIG. 11A), safety studies (FIG. 11B) and fertility studies (FIG. 11C) of Prophetic Example 2.

FIG. 12 shows GALT protein expression via immunohistochemistry (A-D) in wildtype (WT/WT) and GALT null (M3/M3) rats at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. Panel A shows GALT protein staining in liver tissue sections at 14- and 35-days post-treatment with quantification of GALT positive staining. Panel B shows GALT protein staining in skeletal muscle tissue 35 days post-treatment. Panel C shows GALT protein (brown) and astrocyte marker GFAP (purple) co-staining in the cortex of brain tissue 14- and 35-days post-treatment. Panel D shows GALT protein (brown) and either astrocyte marker GFAP (purple) co-staining in the cerebellum of brain tissue 14- and 35-days post-treatment. The administration of AAV9-CAG-hGalt induced GALT enzyme expression in GALT null rat pups.

FIG. 13 is an evaluation of GALT enzyme activity in wildtype (WT/WT) and GALT null (M3/M3) rats liver (A,B), brain (C,D) skeletal muscle (E,F), and eye tissue (G,H) at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. AAV9-CAG-hGalt induced GALT enzyme activity in GALT null rat pups.

FIG. 14 shows the concentration of galactosemia biomarker Gal-1P in liver (A,B), brain (C,D), skeletal muscle (E, F), ovary (G, H), and RBC (I, J) from wildtype (WT/WT) and GALT null (M3/M3) rats at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. Concentration of Gal-1P in the eye (K) at 14 days post-treatment. AAV9-CAG-hGalt significantly reduces Gal-1P levels in the liver, brain, muscle, ovary, RBC, and eye of GALT null rat pups.

FIG. 15 shows the concentration of galactosemia biomarker galactose in liver (A, B), brain (C, D), plasma (E, F), muscle (G, H), ovary (I, J), eye (K, L), and RBC (M, N) from wildtype (WT/WT) and GALT null (M3/M3) rats at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. AAV9-CAG-hGalt significantly reduces galactose levels in the liver, brain, plasma, muscle, ovaries, and eye in GALT null rat pups.

FIG. 16 shows the concentration of galactosemia biomarker galactitol in liver (A,B), brain (C,D), plasma (E,F), muscle (G, H), ovary (I, J), eye (K, L), and RBC (M, N) from wildtype (WT/WT) and GALT null (M3/M3) rats at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. AAV9-CAG-hGalt significantly reduces galactitol levels in the liver, brain, plasma, muscle, ovary, and eyes in GALT null rat pups.

FIG. 17 shows cataract scoring (A,B) and cataract incidence (C) of eyes from wildtype (WT/WT) and GALT null (M3/M3) rats at 14 days and 35 days post-treatment with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. Absence of cataract in an eye is scored as 0 and higher cataract score indicates increased cataract severity. N represents an individual eye. Cataracts were scored at time of sacrifice and therefore the data represent unique animals. AAV9-CAG-hGalt lowers cataract incidence and severity in GALT null rat pups

FIG. 18 shows the daily bodyweight of wildtype (WT/WT) and GALT null (M3/M3) rats from P2 to P37 treated with high dose AAV9-CAG-hGalt (1.16×10¹⁴ vg/kg), low dose AAV9-CAG-hGalt (3.82×10¹³ vg/kg) or AAV9-CAG-hGalt formulation buffer vehicle control. AAV9-CAG-hGalt improves weight gain in GALT null rat pups.

FIG. 19 is a diagram of the experimental plan for in-life and post-life assessments.

### DETAILED DESCRIPTION

### Definitions

When interpreting the description and claims, any one of “comprising,” “consisting of,” “consisting essentially of,” “is selected from the group consisting of,” “is at least selected from the group consisting of,” “is at least one selected from the group consisting of,” and “is, “being,” and “are,” or an equivalent thereof should be understood to contemplate any other and provide support for replacement of that which was recited with any other. For example, when “comprising A, B, or C” is recited, contemplated therein is replacing such, and provides support for embodiments which replace, with: “is A, B, or C,” “consists of A, B, and C,” “consists essentially of A, B, or C,” “is at least selected from the group consisting of A, B, and C,” “is at least one selected from the group consisting of A, B, and C,” or any equivalent thereof.

Further, recitation of “or” contemplates and supports, “one or more of,” “one or a combination of,” or “and,” as in “and/or.” For example, “A, B, or C” contemplates and supports embodiments with: A alone; B alone; C alone; the combination of A and B; the combination of A and C; the combination of B and C; and the combination of A, B, and C. Further to which, within recitation of “closed” language (e.g. consisting of), as well as within recitation of “open” language (e.g. comprising), the recitation of a list, as in “A, B, or C,” contemplates one or a combination within that list, unless otherwise specified. For example, “consisting of A, B, or C” contemplates and supports embodiments with: A alone; B alone; C alone; the combination of A and B; the combination of A and C; the combination of B and C; and the combination of A, B, and C. Recitation of “and/or” contemplates and supports not only the combination of all within the list (i.e. “A, B, and/or C” contemplates “A, B, and C”), but also “one or more of” or “one or a combination of.” For example “A, B, and C” contemplates: A alone; B alone; C alone; the combination of A, B and C; the combination of A and B; the combination of A and C; and the combination of B and C.

The recitation of a list of alternatives with an “and,” as in for example “selected from the group consisting of,” contemplates and provides support for combinations within that list, unless otherwise stated. For example, “is selected from the group consisting of A, B, and C” is to be understood to contemplate and support “is selected from the group consisting of A, B, C, and combinations thereof” and to be coextensive with “is at least one selected from the group consisting of A, B, and C” or such that “group” includes: A alone, B alone, C alone, A and B in combination, A and C in combination, B and C in combination, and A, B, and C in combination.

“Is at least selected from the group consisting of A, B, and C” is contemplated to include embodiments and supports embodiments wherein what is after “is” is open due to recitation of “at least” such it is coextensive with “comprises a member of the group consisting of A, B, and C” or such that “consisting of” modifies the meaning of “group” alone and not “is selected from the group.”

Further, recitation of a component in an embodiment also contemplates and supports exclusion, explicitly, of said component from the embodiment. For example, “comprising A, B, or C” supports embodiments, which comprise A or B, but specifically exclude C.

The articles “a” and “an” are used herein to refer to one or to more than one (i.e., to at least one) of the grammatical object of the article. By way of example, “an element” means one element or more than one element. For example, “comprising an A, a B, or a C” contemplates and supports embodiments comprising two or more A, two or more B, and two or more C.

Unless defined otherwise, all technical and scientific terms have the same meaning as commonly understood by one of ordinary skill in the art to which the embodiments pertain. The preferred materials and methods are described, but it is understood that any methods and materials similar or equivalent to those described can be used in the practice of embodiments. The terminology used herein is for the purpose of describing particular embodiments only and is not intended to be limiting. In describing and claiming the present invention, the following terminology will be used.

“About” as used herein when referring to a measurable value such as an amount, a temporal duration, and the like, is meant to encompass variations of .+−.20% or .+−.10%, more preferably .+−.5%, even more preferably .+−.1%, and still more preferably .+−.0.1% from the specified value, as such variations are appropriate to perform the embodiments.

As used herein, the term “cell line” refers to a population of cells capable of continuous or prolonged growth and division in vitro. Often, cell lines are clonal populations derived from a single progenitor cell. Spontaneous or induced changes can occur in karyotype during storage or transfer of such clonal populations. Therefore, cells derived from the cell line referred to may not be precisely identical to the ancestral cells or cultures, and the cell line referred to includes such variants.

The term “expression” as used herein is defined as the transcription and/or translation of a particular nucleotide sequence driven by its promoter.

As used herein, the term “host cell” refers to any cell that harbors, or is capable of harboring, a substance of interest. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV plasmid encoding a recombinant AAV genome comprising a transgene, an accessory function vector, or other transfer DNA associated with the production of recombinant AAV (rAAV) virions. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

“Identity” as used herein refers to the subunit sequence identity between two polymeric molecules particularly between two amino acid molecules, such as, between two polypeptide molecules or two nucleic acid molecule, such as polynucleotides. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. In the case of an insertion or deletion, identity is understood to realign those thereafter which would be identical and is considered to be not identical at the insertion or deletion.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “nucleic acid,” as used herein, is interchangeable with “polynucleotide” or “a specific sequence of nucleotide.” These terms refer to a discrete sequence that performs a specific function directly or indirectly in a cell. That function includes encoding a sequence of a gene that is transcribed into mRNA and translated into protein and regulating said transcription (i.e. as a promoter would) and/or translation (i.e. as microRNA would). A nucleic acid inherently has a sequence. Thereby, “a nucleic acid comprising SEQ ID NO.: X” can be used to contemplate and support “a nucleic acid comprising the sequence of SEQ ID NO.: X.” In recombinant molecular biology, discrete nucleic acids can be combined. In some embodiments, a nucleic acid that encodes a protein can be ligated to a promoter (which is a nucleic acid), and a cis-acting element of a viral vector (i.e. an inverted-terminal repeat (ITR), which is also a nucleic acid). For convenience, a “nucleic acid” might be used to refer to the discrete elements within the larger nucleic acid, which could be referred to as “a polynucleotide,” “an expression region” (i.e. a polynucleotide comprising a promoter and a nucleic acid that encodes a protein), or “a vector” (see definition below).

“Encoding” refers to the inherent property of a nucleic acid to serve as a template, whether directly (i.e. a sense strand) or indirectly (i.e. an antisense strand) for synthesis of peptide, polypeptides, proteins, or other nucleic acids (i.e. rRNA, tRNA, microRNA). A nucleic acid can “encode” whether it is the sense strand, antisense strand, or a double-stranded segment thereof. The sense strand directly encodes the rRNA, tRNA, microRNA, or mRNA. The mRNA then serves as the template for translation of a peptide, polypeptide, or protein. The anti-sense strand is generally considered to be the reverse complementary sequence and is sometimes called a “non-coding” strand in the art (although for present purposes “non-coding” is a misnomer because the non-coding strand still “encodes” the genetic information by perpetuating it during semi-conservative replication by acting as a template for the polymerization of a new, sense strand). Within semi-conservative replication two single strands in double-stranded nucleic acids are separated, and a new strand is polymerized from the information from each of the single-stranded nucleic acids (i.e. single-stranded template), regardless of whether one single-stranded template is the sense strand (e.g. that which is used to transcribe mRNA and thereby, or directly, encode the translate or protein) or the antisense strand. By perpetuating the genetic information, the antisense strand is still encoding the genetic information for, for example, a protein. Accordingly, “a nucleic acid encoding X”, includes sense and antisense sequences or strands whether X is a peptide, a polypeptide, or a protein or X is a sequence that encodes a rRNA, tRNA, microRNA, antisense RNA, etc.

Further to which, “nucleic acid encoding X,” includes RNA, DNA, and combinations thereof, since nucleic acids are synthesized from transcription, reverse-transcription, and replication, as naturally occurring processes and man-made processes (recombinant biology, molecular biology, etc.).

Accordingly, a recited nucleic acid sequence contemplates and supports the complementary version thereof, the reverse complementary version thereof, and double-stranded versions thereof. That is, “a nucleic acid comprising SEQ ID NO.: X” is to be understood, contemplate, and support “a nucleic acid comprising the reverse complementary of SEQ ID NO.: X” or, using the nomenclature regarding the prime symbol as in “′”, “a nucleic acid comprising SEQ ID NO.: X′,” unless otherwise specified. For example, “the nucleic acid comprising SEQ ID NO.: X” wherein SEQ ID NO.: X is 5′-ATGCC-3′ contemplates and supports the reverse complementary of SEQ ID NO.:X, and specifically 5′-GGCAT-3.

As noted above, a recited nucleic acid sequence contemplates and supports conversion between RNA and DNA versions thereof. For example, if SEQ ID NO.: X is “5′-ATGCC-3′,” contemplated and supported is 5′-AUGCC-3′, as well as the reverse complementary thereof, 5′-GGCAU-3.

With regard to an AAV vector or an AAV virion, the above-noted incorporation of reverse complementary sequences and double-stranded segments into the definition of “a nucleic acid” and the above-noted use of “encoding” as including sense and antisense strands, is intended to incorporate the means by which the AAV vector can introduce an exogenous nucleic acid sequence that encodes nucleic acid or a protein into the cell. It is further intended to incorporate, in some embodiments, processes whereby said introduction results in the expression of said nucleic acid (i.e. miRNA or antisense RNA) or protein (i.e. galactose-1-phosphate uridylyltransferase (GALT)).

Take for example, a nucleic acid encoding a protein, and an AAV vector comprising a nucleic acid encoding said protein. When a typical (i.e. naturally occurring) AAV vector encoding one sense or one antisense strand of the nucleic acid that encodes said protein enters the cell, the inverted-terminal repeats (ITRs) prime the synthesis of a sequence reverse complementary to the sense strand or antisense strand of the nucleic acid that encodes said protein. The polymerization thereby forms a segment of double-stranded DNA comprising the sense and antisense strands, regardless of whether the sense version or antisense version was first introduced to the cell. In this regard, the entire nucleic acid including ITRs and sense and antisense nucleic acids encoding a protein can be one single-stranded DNA, which loops upon itself to form a double-stranded segment, wherein the base-pairs the sense and antisense nucleic acids encoding the protein align.

From this segment of double-stranded DNA, transcription of mRNA and translation of said protein is achieved from said sense strand of DNA, regardless of whether the AAV vector comprised only the sense strand or only the antisense strand when first entering the cell. In this regard, “an AAV vector comprising a nucleic acid encoding protein X” includes, contemplates, and supports embodiments in which the nucleic acid is the sense strand encoding protein X, the antisense strand encoding protein X, a double-stranded nucleic acid encoding protein X, and a single stranded nucleic acid comprising sense and antisense strands wherein the sense and antisense strands form a segment of double-stranded nucleic acid.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

The term “promoter” as used herein is defined as a DNA sequence recognized by the synthetic machinery of the cell, or introduced synthetic machinery, required to initiate the specific transcription of a polynucleotide sequence. In some instances, this sequence may be the core promoter and in other instances, this sequence may also include, or be an enhancer alone and/or other regulatory elements which are required for expression of the gene product.

In certain instances the promoter may comprise enhancer elements, exons, and introns from one or a variety of viruses and animals, and thereby the term “promoter” shall be understood to not be limited to being a non-expressed sequence, nor exclude a non-expressed sequence that is between expressed sequences (i.e. introns), nor be limited to exclude an enhancer alone so long as the combination of sequences used to construct the promoter are capable of initiating the specific transcription of a polynucleotide sequence.

A “constitutive” promoter is a nucleotide sequence which, when operably linked with a polynucleotide that encodes or specifies a gene product, causes the gene product to be produced in a cell under most or all physiological conditions of the cell and without requiring the addition of exogenous factors or the introduction of a different phenotype to the cell. This constitutive promoter can be cell-specific so long as it is produced in the specific, or target, cell under most or all physiological conditions of the cell. For example, a telencephalic neuronal-specific promoter is calcium/calmodulin-dependent protein kinase II (CaMKII). The CAG promoter and an elongation factor 1 (EF1) promoter are examples of constitutive promoters in a broad range of target cell types.

An “inducible” promoter is a nucleotide sequence which, when operably linked with a polynucleotide which encodes or specifies a gene product, causes the gene product to be produced in a cell substantially only when an inducer which corresponds to the promoter is present in the cell. For example, the promoter in a cyclooxygenase-2 gene is considered to be an inducible promoter in the periphery.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

A “target gene” refers to a nucleic acid encoding a target protein to be expressed within a target cell upon entry of the vector carrying the target gene into the cell. The target gene includes naturally occurring polymorphisms (i.e. variants) and man-made modifications to the wild-type gene so long as the target protein is still expressed. An example of such man-made modifications includes codon-optimization.

A “target protein” refers to a man-made or naturally occurring protein of interest to be introduced by vector into a host cell. One some embodiments, the target protein, as encoded in the genome of the host cell, is not functional because of a polymorphism in the gene sequence resulting in some mistranscription, missense, or mistranslation of the gene whereby reduced or no target protein or inoperable target protein is produced (i.e. a polymorphism results in an early stop codon) or an attenuation in the activity of the target protein, as encoded by and expressed from the genome of a subject.

In some embodiments, the target protein comprises galactose-1-phosphate uridylyltransferase (GALT). GALT is the enzyme that interconverts (i.e. a reversible enzymatic reaction) uridine diphosphate-glucose and galactose-1-phosphate into glucose-1-phosphate and uridine diphosphate-galactose (i.e. the second step of the Leloir pathway of galactose metabolism). It is to be understood and contemplated that “GALT” encompasses naturally-occurring versions (i.e. human GALT) and non-naturally occurring GALT (i.e. amino-acid additions, deletions, or substitution of GALT, which increase or decrease the activity compared to that of naturally occurring GALT) so long as the enzyme referred to as GALT has at least the above-noted enzymatic activity. In some embodiments, the non-naturally occurring GALT has at least, or no more than, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the activity of the corresponding naturally-occurring GALT, wherein “corresponding” contemplates and provides support for that to which the additions, deletions, or substitutions were applied. In embodiments, the GALT is a human GALT and, in some embodiments, has the amino acid sequence of SEQ ID NO:1. In alternate embodiments, the GALT has an amino acid sequence that has at least 99%, 95%, 90%, 85% or 80% sequence identify to SEQ ID NO:1 and has GALT activity. In other embodiments, the hGALT is encoded by the nucleotide sequence of SEQ ID NO: 2, or a nucleotide sequence that is at least 99%, 95%, 90%, 85% or 80% identical to SEQ ID NO:2 and encodes hGALT having the amino acid sequence of SEQ ID NO: 2 or an hGALT which has an amino acid sequence that has at least 99%, 95%, 90%, 85% or 80% sequence identify to SEQ ID NO:1 and has GALT activity

In some embodiments, the subject is deficient in GALT, as encoded by and expressed from the genome of the subject, due for example to an autosomal recessive inheritance of two defective GALT genes. In some embodiments, the subject has classic galactosemia. In some embodiments, the subject has an attenuated activity in GALT. In some embodiments, the subject has Duarte galactosemia. In some embodiments, the subject has an unstable form of the GALT enzyme, whether it is a polymorphism causing lower activity in the enzyme in comparison to that of individuals who do not have the polymorphism. In some embodiments, the subject has a mutation in the promoter regulating GALT transcription, which causes reduced transcription of mRNA encoding GALT and thereby, potentially, reduced expression of GALT (i.e. reduced GALT protein). In some embodiments, the subject has the combination of one gene encoding a deficiency in GALT activity and another gene encoding an attenuation in GALT activity. That is in some embodiments, the subject's genotype is heterozygous for the classical variant and the Duarte variant. In some embodiments the subject has one or both of a mutation from A to G in exon 6 of the GALT gene, which changes Glu188 to an arginine, and a mutation from A to G in exon 10, which changes Asn314 to an aspartic acid.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “vector” is a nucleic acid capable of delivering a target gene to the interior of a cell, and includes not only the expression-region (i.e. a promoter and a nucleic acid encoding a protein or even a nucleic acid), but also some cis-acting genetic component. The cis-acting genetic component provides for packaging within a virion, expression in a cell, replication in a cell, or a combination thereof.

By way of example, inverted-terminal repeats (ITRs) from adeno-associated viruses (AAVs) constitute a vector when adjoined to the nucleic acid encoding a target protein because the ITRs will provide for the nucleic acid encoding the target protein to be packaged within an AAV virion. ITRs also provide other cis-acting functions for expression of the nucleic acid encoding the target protein in the host cell upon entry of the vector into the host cell. Such cis-acting functions of ITRs include aiding in concatemer formation for genomic insertion; initiation of second strand formation in the case of a single-stranded (ss) AAV (ssAAV) vector; or initiation of replication and transcription in the case of ssAAV and self-complementary (sc) AAV (scAAV) vectors. In this regard, the AAV ITRs can be characterized based on the nucleic acid sequences providing such cis-acting functions from the serotypes of AAVs. That is, an ITR isolated from an AAV2 serotype can be known as an AAV2 ITR, even though the ITR generally does not contribute to the serotype of an AAV.

Further it is understood that although the scAAV ITR (e.g. SEQ ID NO.:13) was developed by mutating or altering a wild type AAV2 ITR (e.g. SEQ ID NO.: 11, which is the 5′ flanking ITR) and specifically the terminal resolution site (trs) in the D-sequence, which is responsible for signaling for packaging (a packaging sequence), the scAAV ITR provides for a function not provided for by the wild-type AAV ITR, which is the generation of an AAV vector comprising an expressing region and a reverse complement thereof prior to packaging within the AAV virion. Without wishing to be bound to a particular theory, it is believed that when the producing cells express the AAV vector plasmid, an AAV vector comprising the expressing region and the two ITRs, one of which is the scAAV ITR, is produced. Because of the negating mutation to the trs in the D-sequence, DNA polymerase is able to polymerize from the expressing region and the remaining ITR their reverse complements. The scAAV vector is thereby obtained. In some embodiments, the scAAV vector is then packaged into a recombinant scAAV virion.

The scAAV vector may exhibit at least, or no more than, 50%, 60%, 70%, 80%, 90%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 110-fold, 120-fold, 130-fold, or 140-fold more efficient transduction than a corresponding vector comprising a wild-type AAV2 ITR and no complement of the expressing region. Without wishing to be bound to a particular theory, it is believed that the host-cell synthesis of a double-stranded segment in a ssAAV vector is rate limiting in ssAAV vector transduction, and that by providing an expressing region and the reverse complement thereof said scAAV ITR (and vector comprising said scAAV ITR) provides for the above-noted increase in efficiency of transduction.

By way of example, a plasmid can comprise an origin of replication (e.g., on from cytomegalovirus) which allows for the replication of the target gene within a cell, and such a plasmid is thereby a vector. A viral genetic code may provide a nucleic acid sequence or protein encoded therein that allows for insertion of the gene of interest into the host genome, thereby providing for the replication of the target gene during the replication of, and within, the host cell's genome.

“Expression vector” refers to a vector comprising an expressing region. An expressing region includes a recombinant polynucleotide comprising a nucleic acid that controls expression (i.e. a promoter) and a nucleic acid that encodes. The nucleic acid that encodes includes a nucleic acid that encodes a protein. Generally, the promoter is operatively linked to the nucleic acid that encodes the target protein in a manner that is capable of promoting expression of the protein upon entry of the vector into the host cell. In some embodiments, the promoter can be operably linked by ensuring that there is not codon misalignment.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The nucleotide and amino acid sequences provided herein are set out in Table 1.

### AAV Vectors

In some embodiments, a recombinant adeno-associated virus (AAV) vector is provided, which comprises an expressing region and at least two inverted-terminal repeats (ITRs); the expressing region comprises a promoter and a nucleic acid that encodes a galactose-1-phosphate uridylyl transferase (GALT); the promoter is operably linked to the expression of GALT; and the at least two ITRs flank the expressing region, the expressing region may further include other regulatory sequences such as enhancers, polyA signals, intron sequences and WPRE sequences.

In some embodiments, the GALT comprises an amino acid sequence comprising at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; the amino acid sequence of SEQ ID NO.: 1. In some embodiments, the GALT comprises the amino acid sequence of SEQ ID NO.: 1. In some embodiments, the nucleic acid that encodes GALT comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 2 or a reverse complementary sequence thereto. In some embodiments, the nucleic acid that encodes GALT comprises or consists of SEQ ID NO.: 2 or a reverse complementary sequence thereto.

In some embodiments, the ITR, or at least two ITRs, comprises an AAV1 ITR, AAV2 ITR, AAV3 ITR, AAV4 ITR, AAV5 ITR, AAV6 ITR, AAV7 ITR, AAV8 ITR, or an AAV9 ITR, and in embodiments may be an scAAV ITR. In some embodiments, the ITR, or at least two ITRs, comprises a AAV2 ITR or a scAAV ITR. In some embodiments, the ITR, or at least two ITRs, comprises AAV2 ITR and a scAAV ITR. In some embodiments, the scAAV ITR is an AAV2 ITR lacking at least one functional terminal resolution site (trs) in the D-sequence. In some embodiments, the scAAV ITR lacks at least one functional trs. In some embodiments, the scAAV ITR has at least one substitution, addition, or deletion in the at least one trs, wherein the at least one substitution, addition, or deletion confers lack of function to the at least one trs. In some embodiments, the AAV ITR has at least one a D-sequence deleted. In some embodiments, the D-sequence deleted is at the 3′ end of the ITR (ssD[−]). In some embodiments, the D-sequence deleted is a the 5′ end of the ITR (ssD[+]). In some embodiments, the ssD[−] sequence has at least one substitution, deletion, or addition that prevents binding of the 52-kDa-FK506-binding protein (FKBP52). In some embodiments, the AAV ITR has a D-sequence replaced with a transcription factor binding site. In some embodiments, the transcription factor binding site comprises an S-sequence. In some embodiments, the S-sequence comprises a Foxd3 binding site or a NF-pE1 binding site. In some embodiments, the transcription factor binding site or the S-sequence comprises a GATA-1 and GATA-2 binding site. In some embodiments, the ITR, or at least two ITRs, comprise a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 11, SEQ ID NO.: 13, or a reverse complementary sequence thereto, including SEQ ID NO: 12. In some embodiments, the ITR, or at least two ITRs, comprise SEQ ID NO.: 11, SEQ ID NO.: 12, or a reverse complementary sequence thereto. In fact the flanking ITRs may be reverse complements of each other such that the ITR at the 5′ end of the expression cassette has a nucleotide sequence of SEQ ID NO: 11 and the ITR at the 3′ end of the expression cassette has a nucleotide sequence of SEQ ID NO: 12 (or the reverse complement of each). In the case of a self-complementary vectors, the ITR at the 5′ end of the expression cassette has a mutant sequence of SEQ ID NO:13 and the ITR at the 3′ end of the expression cassette is an unmodified ITR, for example, and AAV2 ITR having a nucleotide sequence of SEQ ID NO: 11 at the 5′ end of the expression cassette.

In some embodiments, the sequence encoding GALT is operably linked to a promoter, including a constitutive promoter. In some embodiments the promoter is a CAG promoter. A CAG promoter is a composite, synthetic promoter which contains the CMV early enhancer element, the chicken β-actin promoter and the first exon and first intron of the chicken β-actin gene and the splice acceptor of the rabbit β globin gene. See, e.g., Miyazaki et al, Gene 79:269-277 (1989) and Niwa et al, Gene 108:193-199 (1991). In certain embodiments, the CAG “promoter” has a nucleotide sequence of SEQ ID NO:9, or may be an at least 200, 300, 400, 500 or 600 nucleotide fragment thereof with promoter activity that promotes expression of the target gene in the appropriate tissues (or a reverse complement thereof as appropriate). In other embodiments, the promoter is an EF1a or EF1α promoter, which may or may not include the EF-1α intron A sequence. In certain embodiments, the EF1a or EF1α promoter has a nucleotide sequence of SEQ ID NO: 10 (or a nucleotide sequence of the first 230 nucleotides of SEQ ID NO: 10, which does not include the EF1α intron A. Alternately, the EF1α promoter is an at least 100, 200, 300, 400, 500, 600, 700, 800, 900 or 1000 nucleotide fragment of SEQ ID NO:10 and has promoter activity that promotes expression of the target gene in the appropriate tissues (or a reverse complement thereof as appropriate). In some embodiments, the promoter comprises a nucleic acid sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 9, SEQ ID NO.: 10, or a reverse complementary sequence thereto and promotes GALT gene expression in the appropriate tissues. In some other embodiments, the promoter comprises or consists of a Rous sarcoma virus (RSV) LTR promoter, a cytomegalovirus (CMV) promoter, a simian virus (SV40) promoter, a dihydrofolate reductase promoter, a β-actin promoter, a phosphoglycerol kinase (PGK) promoter, a P5 promoter, a Ubc promoter, a tetracycline response element promoter, a UAS promoter, an Ac5 promoter, a polyhedrin promoter, a calmodulin-dependent protein kinase II-α (CaMKIIα) promoter, a galactose promoter, the GALT promoter, a GDS promoter, an alcohol dehydrogenase promoter, an H1 promoter, a U6 promoter, or an Alpha-1-antitrypsin promoter. In some embodiments, the β-actin promoter is a chicken β-actin (“CBA”) promoter or a human β-actin promoter.

In some embodiments, the expressing region is in an anti-sense (e.g. reverse complementary) orientation or in sense orientation. In some embodiments, the vector comprises two or more expressing regions. In some embodiments, the two or more expressing regions comprises one in antisense orientation and another in sense orientation (i.e. as an scAAV would).

In some embodiments, the expressing region further comprises a nucleic acid that encodes a polyadenylation (poly(A)) signal 3′ of the target gene coding sequence such that the expressed mRNA has a polyA tail. In some embodiments, the nucleic acid that encodes the poly(A) signal comprises a bovine growth hormone (bGH) poly(A) tail signal or a simian virus 40 (SV40) poly(A) tail signal. In some embodiments, the polyA signal has a nucleotide sequence of SEQ ID NO 15 (bGH polyA signal) or SEQ ID NO: 16 (SV40 polyA signal) (or a reverse complement thereof) or the polyA signal has at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 15, SEQ ID NO.: 16, or a reverse complementary sequence thereto.

In some embodiments, the expression cassette further comprises regulatory elements that may enhance the expression of the target gene. In one embodiment, the expressing region or expression cassette further comprises a woodchuck hepatitis virus post-transcriptional regulatory element (WPRE). In some embodiments, the WPRE is downstream (3′ of) the GALT coding sequence and upstream (5′ of) the poly(A) tail signal. In some embodiments, the WPRE comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO.: 14 (or reverse complement thereof) that enhances target gene expression. In some embodiments, the WPRE comprises SEQ ID NO.: 14 (or a reverse complement thereof). In other embodiments, the expression cassette comprises an intron or a chimeric intron sequence that promotes target gene expression. The intron sequence may be inserted between the promoter and the hGALT coding sequence. In certain embodiments, the intron has a nucleotide sequence of SEQ ID NO: 17 (or reverse complement thereof), or a nucleotide sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO.: 17 (or reverse complement thereof) that enhances target gene expression

In some embodiments, the recombinant AAV vector may be packaged into a recombinant AAV virion.

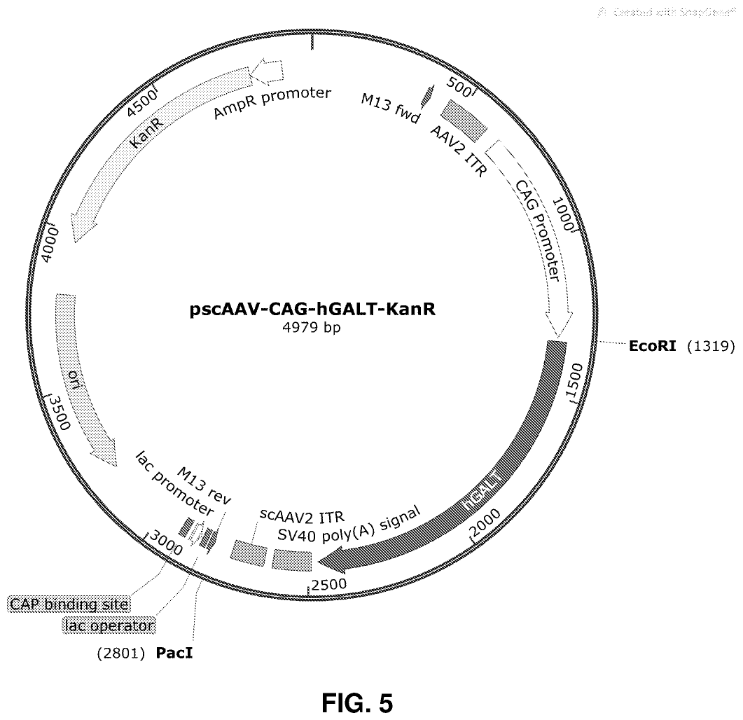

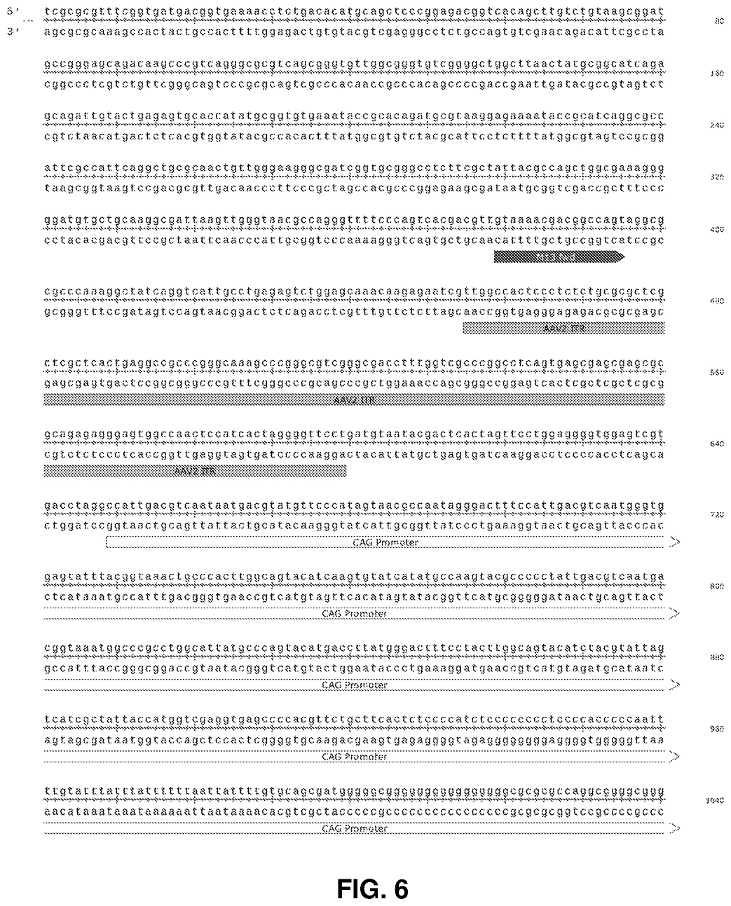

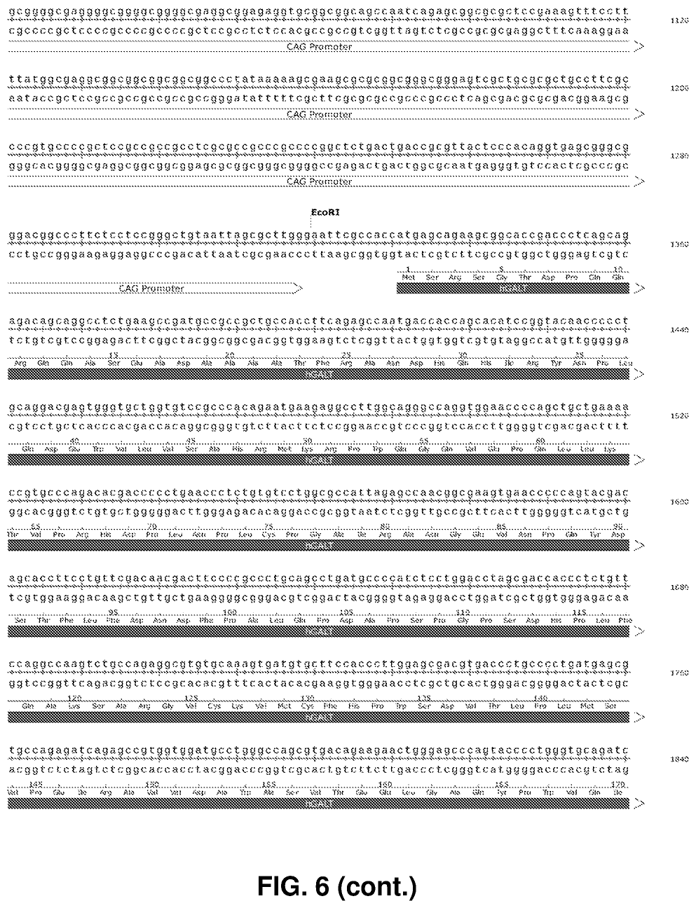

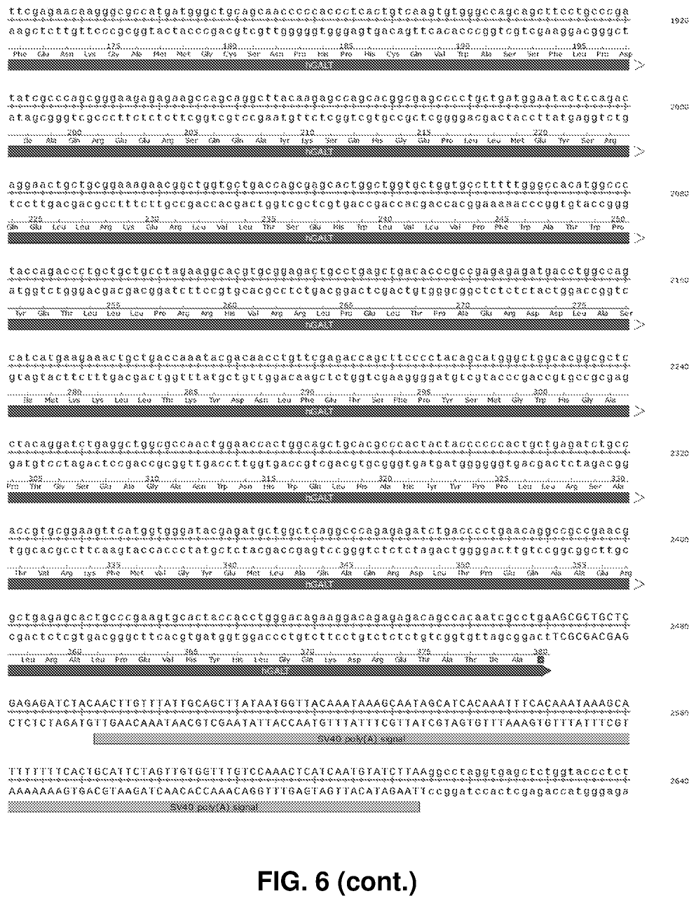

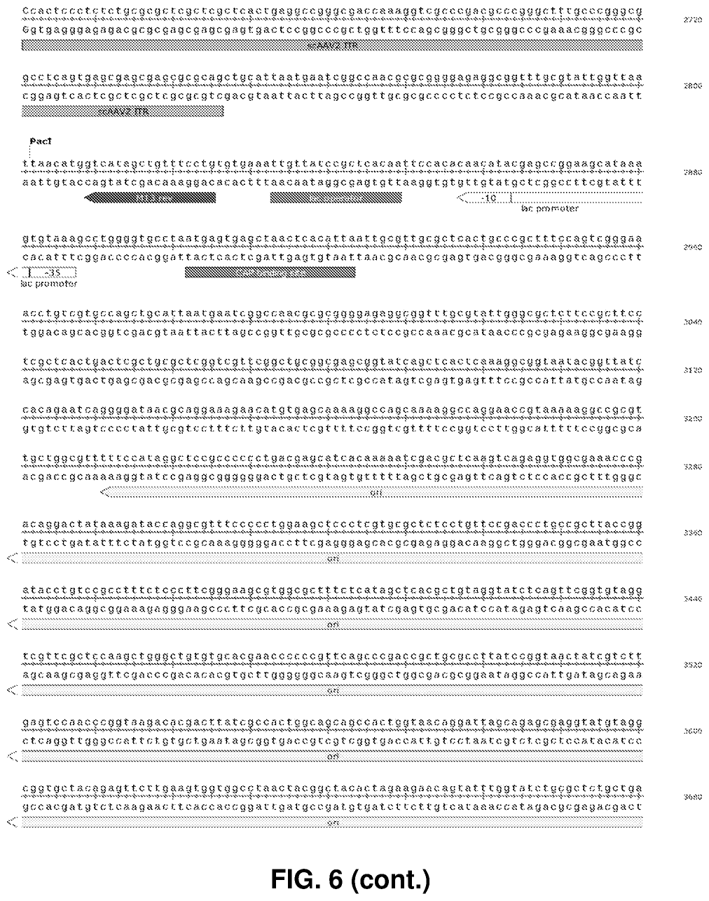

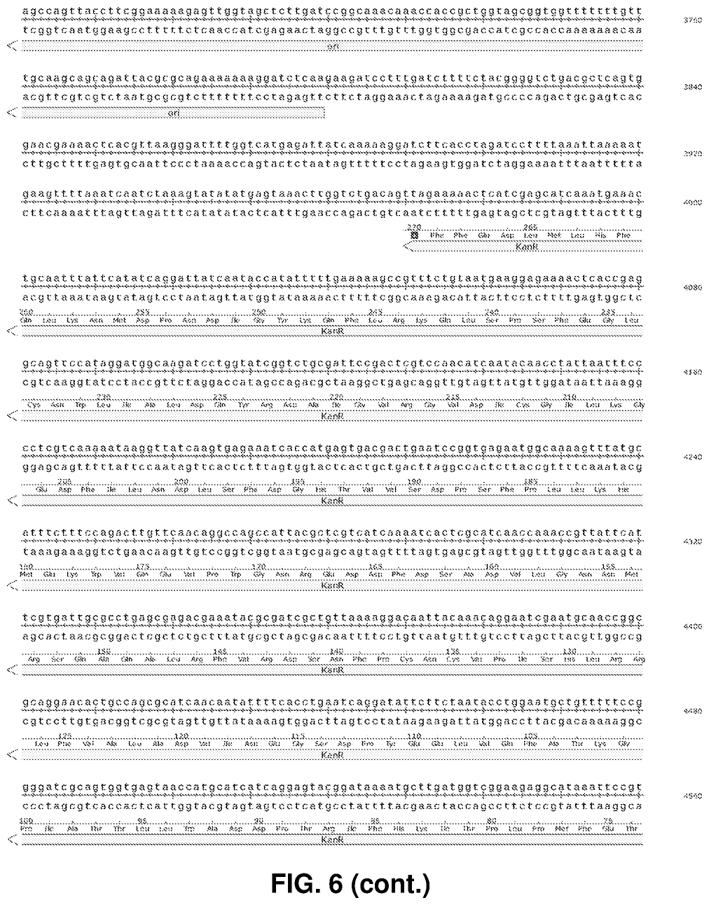

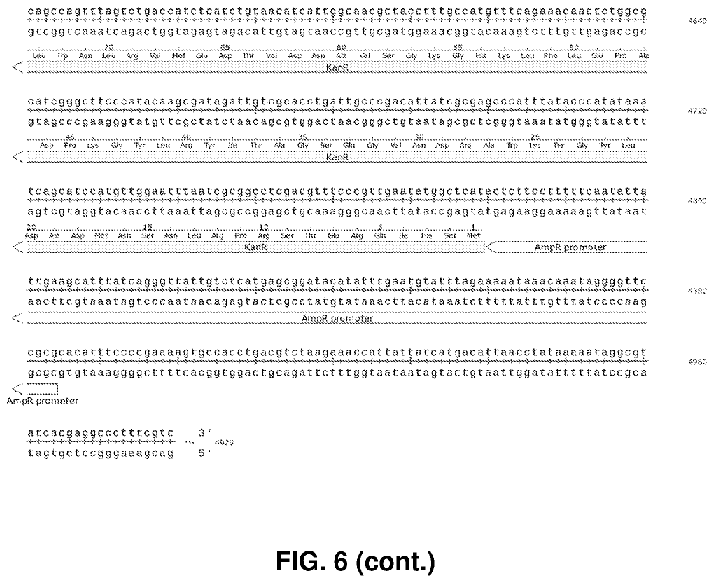

In some embodiments, a recombinant self-complementary AAV (scAAV) vector is provided. In some embodiments, the scAAV comprises the expressing region, at least three ITRs, and a nucleic acid reverse complementary to expressing region. In some embodiments, the at least three ITRs comprise at least one scAAV ITR. In some embodiments, the scAAV vector is in the following order: at least one of the three ITRs, the expressing region, the scAAV ITR, the nucleic acid reverse complementary to the expressing region, and at least one of the three ITRs. In some embodiments, the at least three ITRs comprise at least two scAAV ITRs. In some embodiments, the scAAV vector is in the following order: at least one of the at least two scAAV ITRs, the expressing region, at least one of the ITRs, the nucleic acid reverse complementary to the expressing region, and at least another of the at least two scAAV ITRs. In some embodiments, a scAAV vector plasmid is provided; the scAAV plasmid encoding the scAAV. In some embodiments, the scAAV vector plasmid encoding the scAAV will comprise at least two ITRs and the expressing region, wherein at least one of the at least two ITRs is the scAAV ITR. In embodiments, as discussed above, the 5′ ITR may have a nucleotide sequence of SEQ ID NO:11 and the modified ITR may be at the ′3 end of the expression cassette and have a nucleotide sequence of SEQ ID NO: 13. FIGS. 5 and 6 depict a plasmid and its nucleotide sequence, respectively, which can produce an scAAV.

Provided, thus, are expression cassettes that can be incorporated into an AAV vector for gene replacement expression of the target gene. In particular embodiments, the expression cassette may have elements arranged as follows: 5′AAV2ITR-CAG promoter sequence-hGALT coding sequence-SV40 polyA signal sequence-3′scAAV2 ITR. In embodiments, the expression cassette has a nucleotide sequence of SEQ ID NO: 3. In other embodiments, the gene expression cassette has elements arranged as follows: 5′AAV2ITR-CAG Promoter (CMV enhancer-CBA promoter-Intron sequence)-hGALT coding sequence-WPRE sequence-bGH polyA signal sequence-3′AAV2ITR sequence. In embodiments, the expression cassette has a nucleotide sequence of SEQ ID NO:4 (or the reverse complement thereof). In another embodiment, the expression cassette may have elements arranged as follows: 5′ITR-EF1α promoter sequence-hGALT coding sequence-WPRE sequence-bGH polyA signal sequence-3′AAV2 ITR. In embodiments, the expression cassette has a nucleotide sequence of SEQ ID NO: 5 (or reverse complement thereof). In embodiments, the expression cassette has a nucleotide sequence of SEQ ID NO:5 (or the reverse complement thereof).

In some embodiments, the scAAV vector is then packaged into a recombinant AAV virion.

In some embodiments, the expressing region comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 3, SEQ ID NO.: 4, SEQ ID NO.: 5, SEQ ID NO.: 19, SEQ ID NO.: 20, or SEQ ID NO.: 21 or a reverse complementary sequence thereto, and is an expression cassette that expresses hGALT in appropriate human tissues. In some embodiments, the expressing region or expression cassette comprises SEQ ID NO.: 3, SEQ ID NO.: 4, SEQ ID NO.: 5, SEQ ID NO.: 19, SEQ ID NO.: 20, or SEQ ID NO.: 21 or a reverse complementary sequence thereto.

### AAV Vector Plasmids

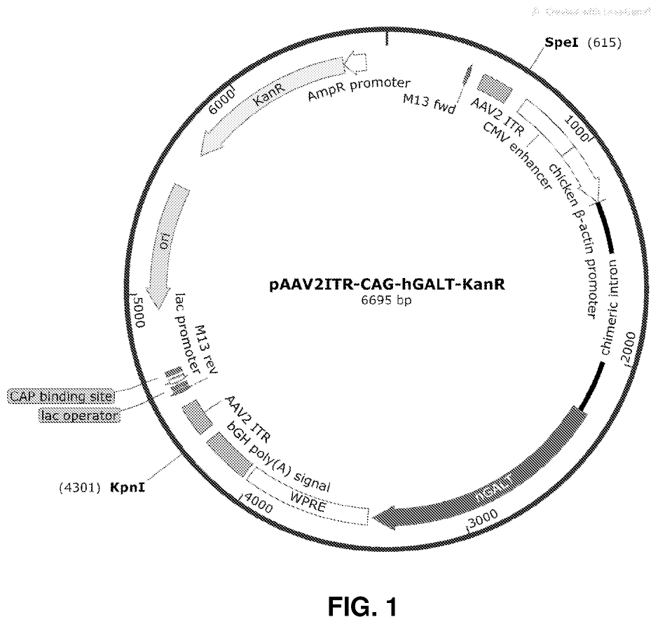

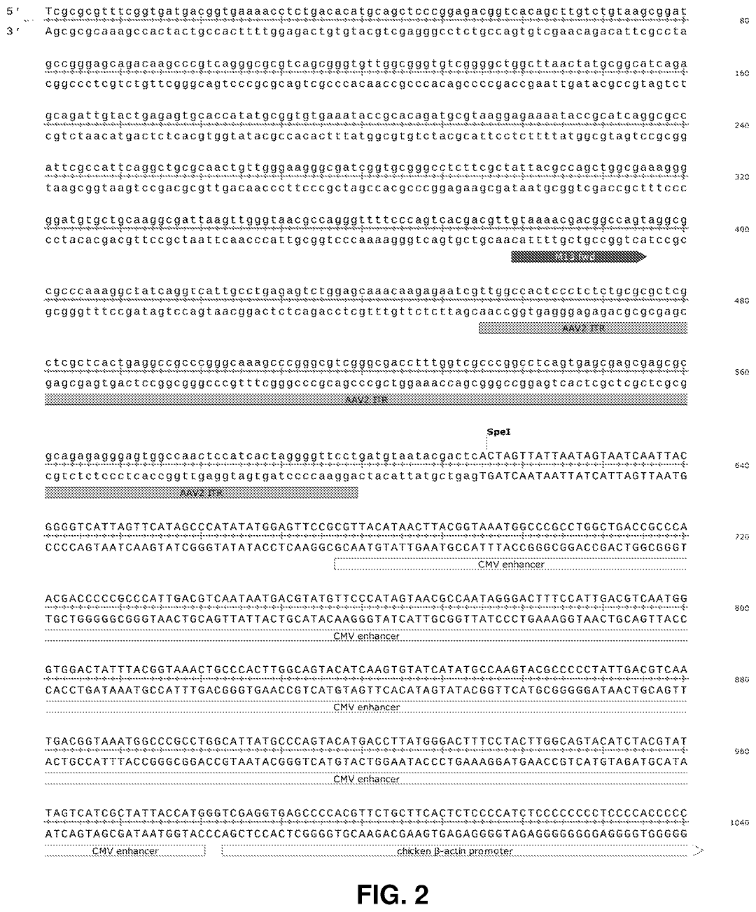

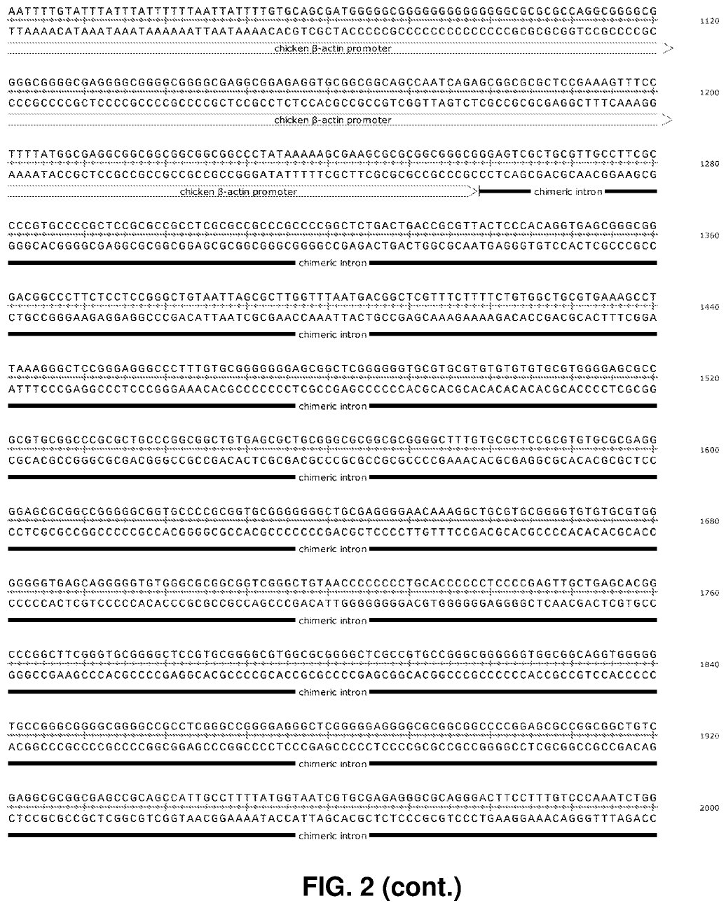

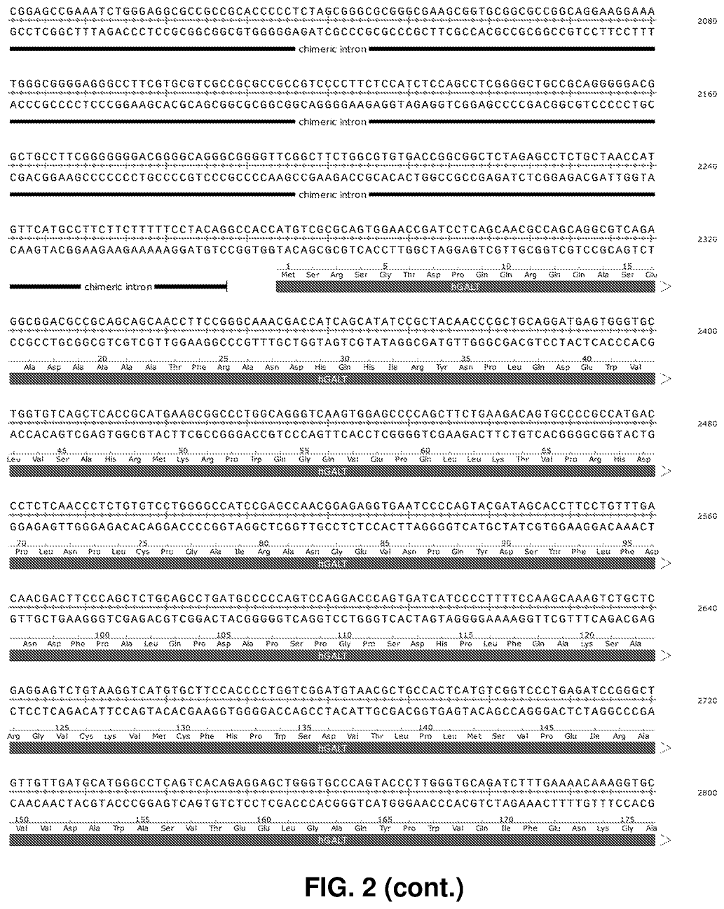

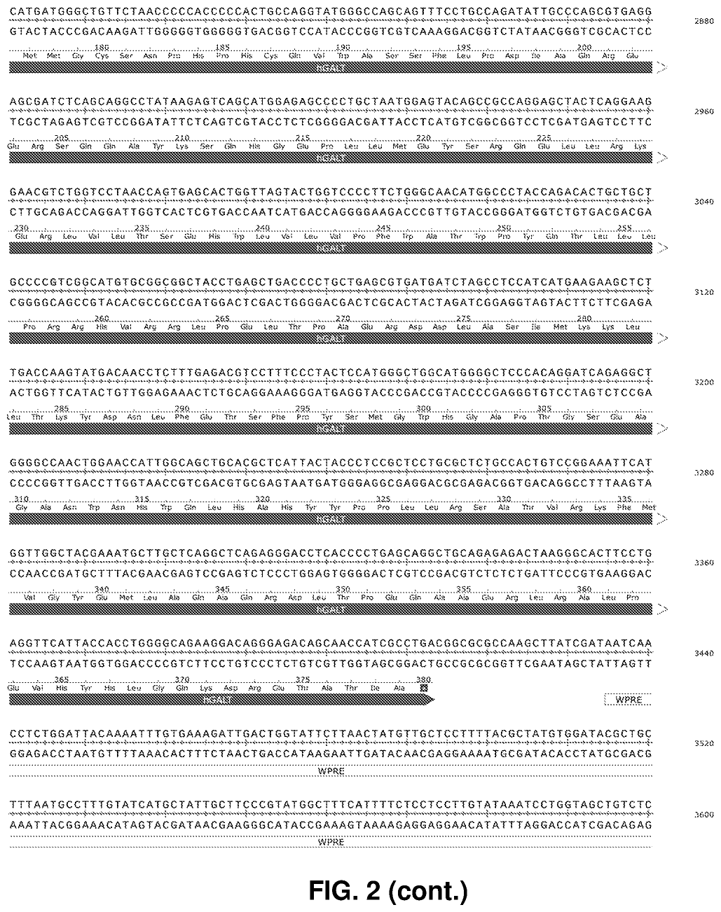

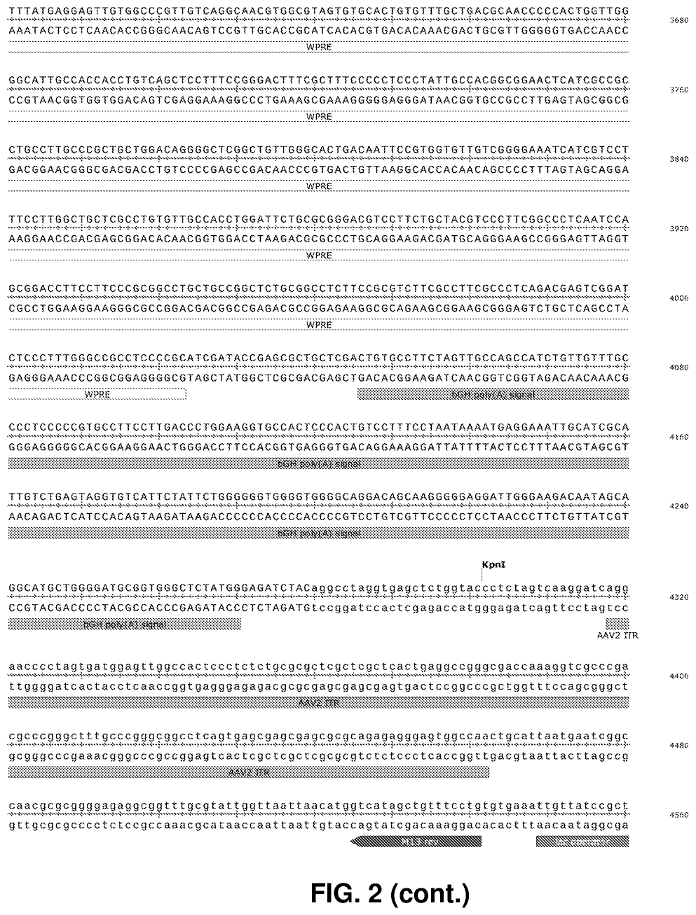

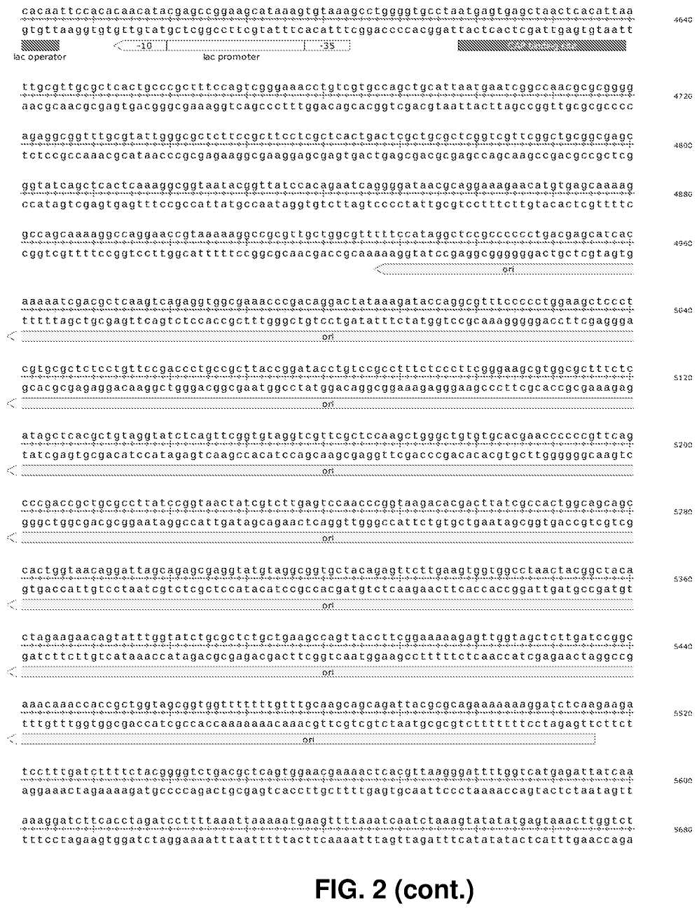

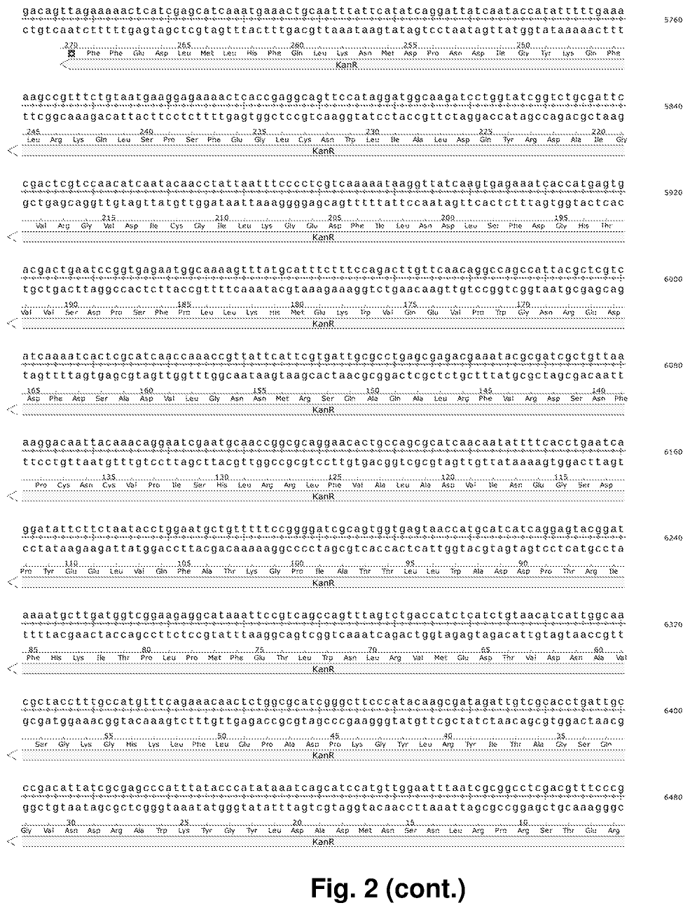

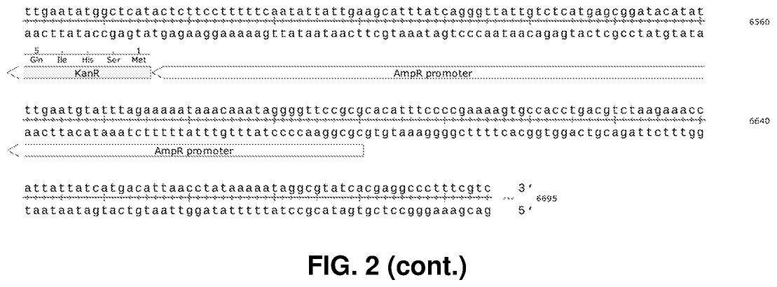

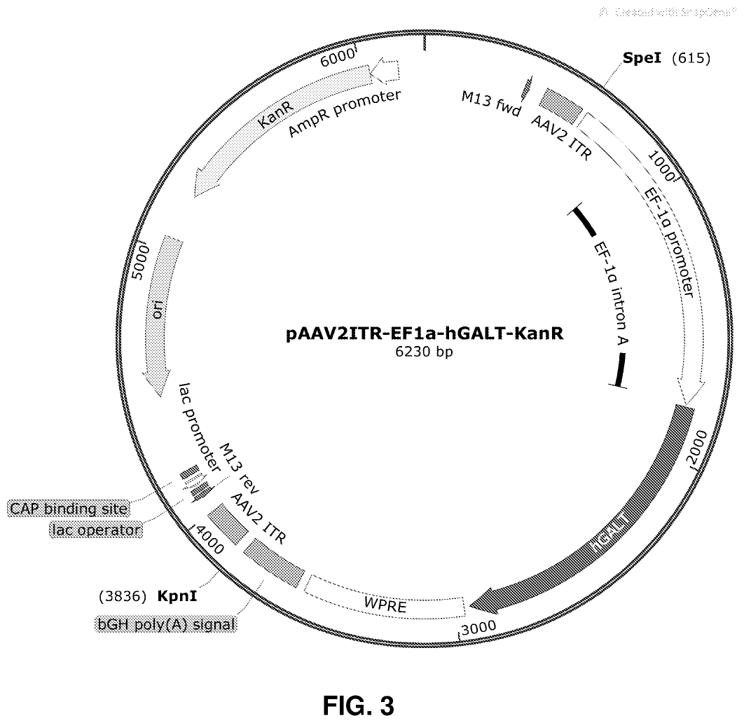

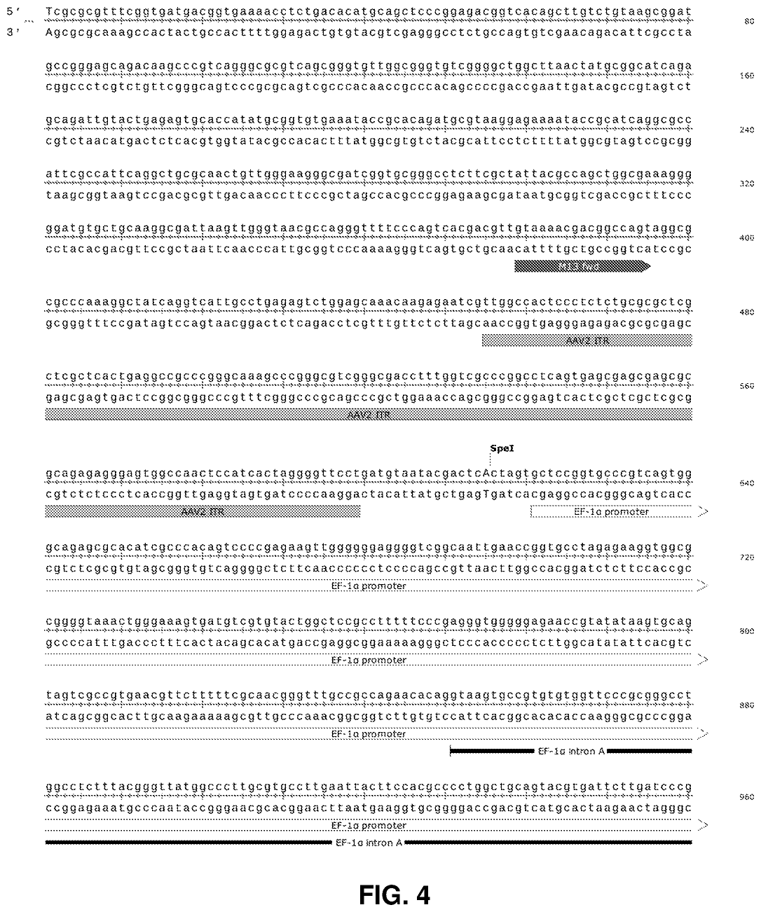

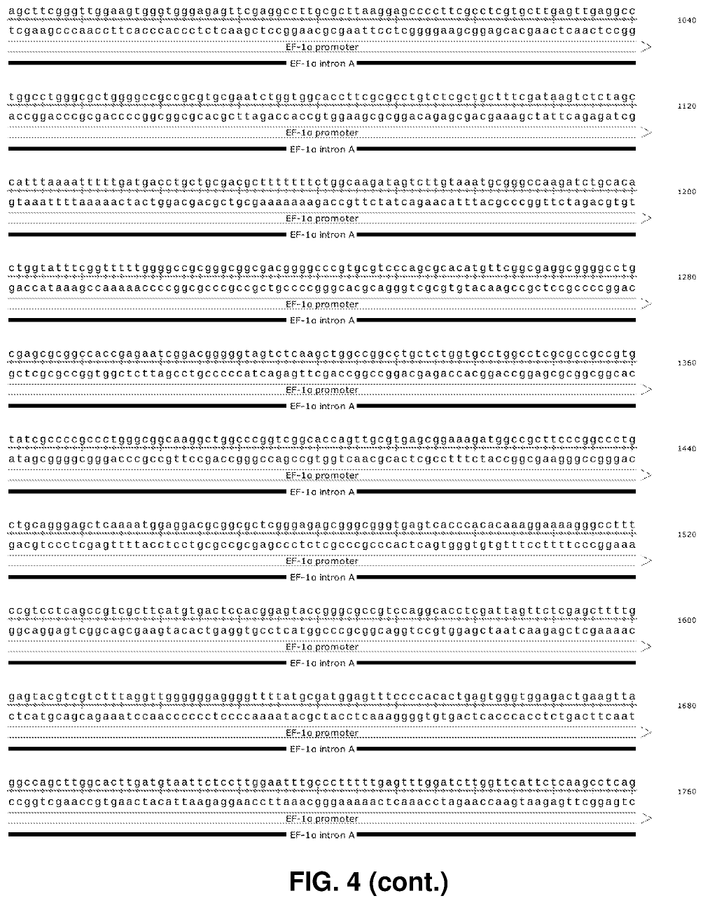

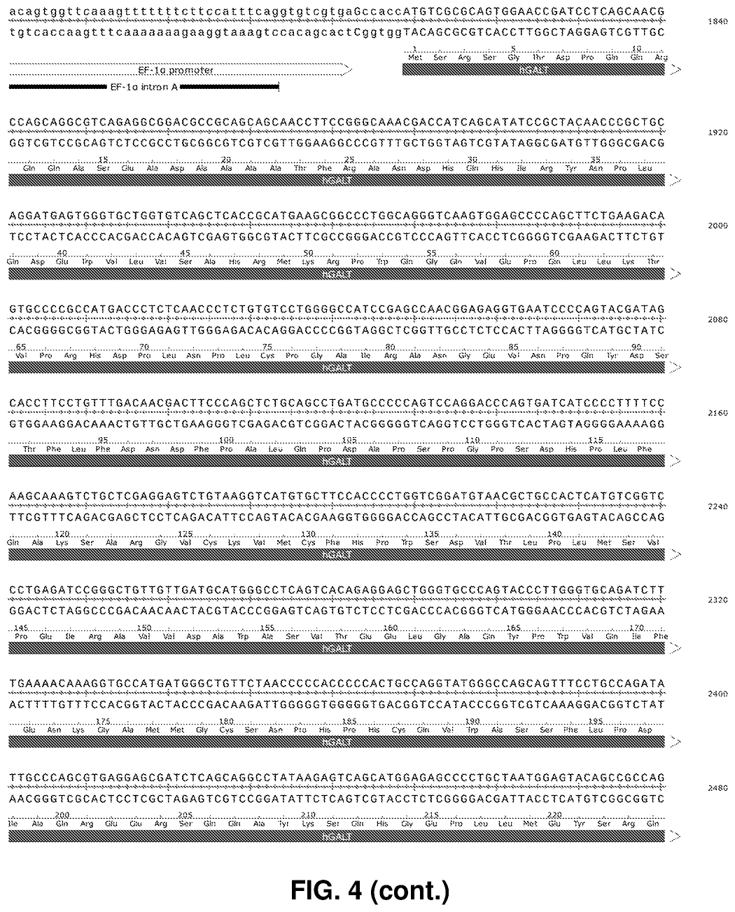

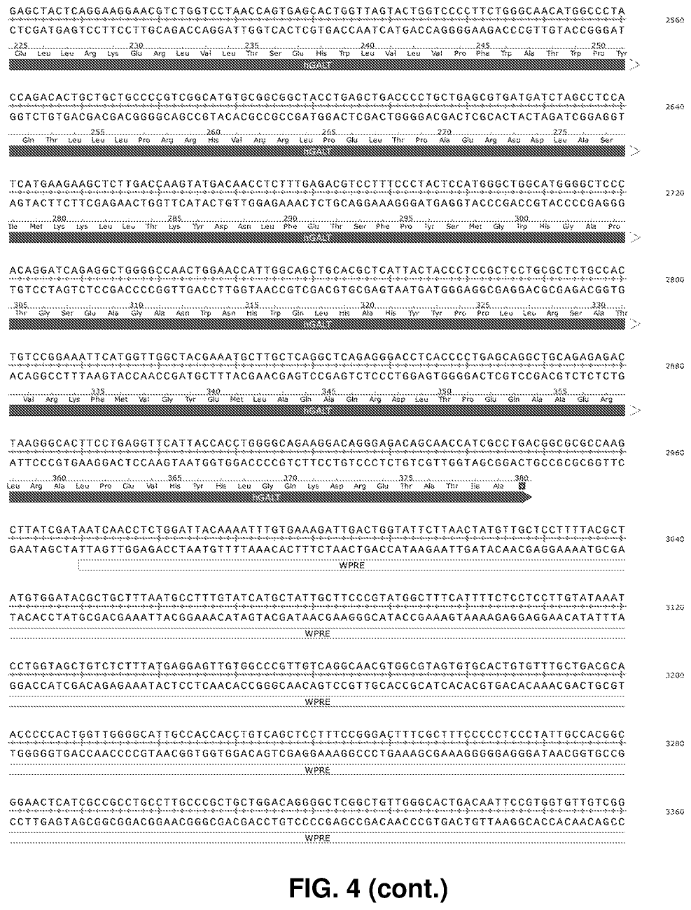

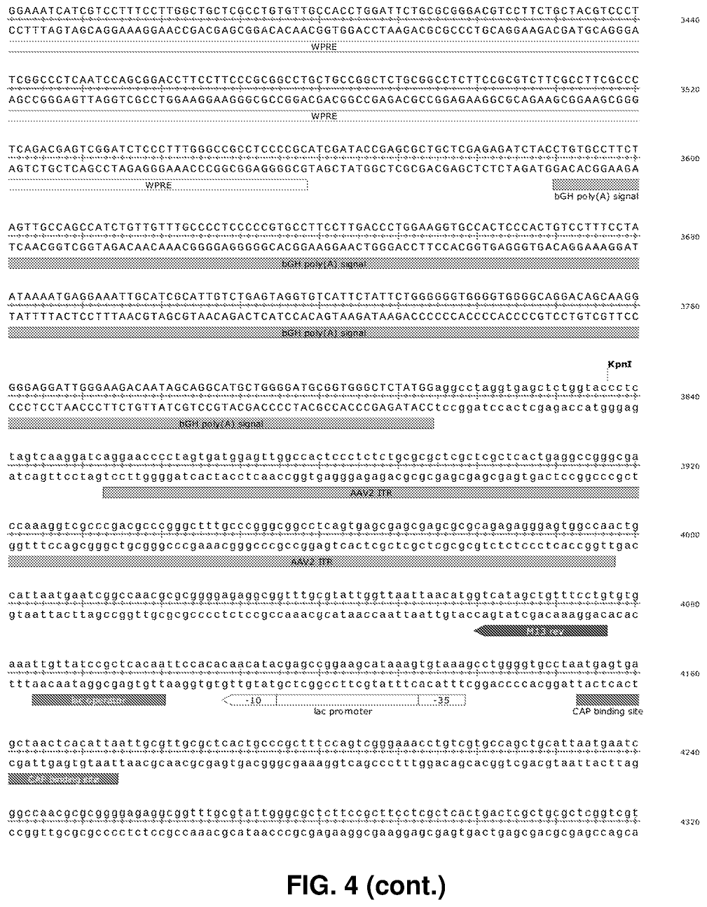

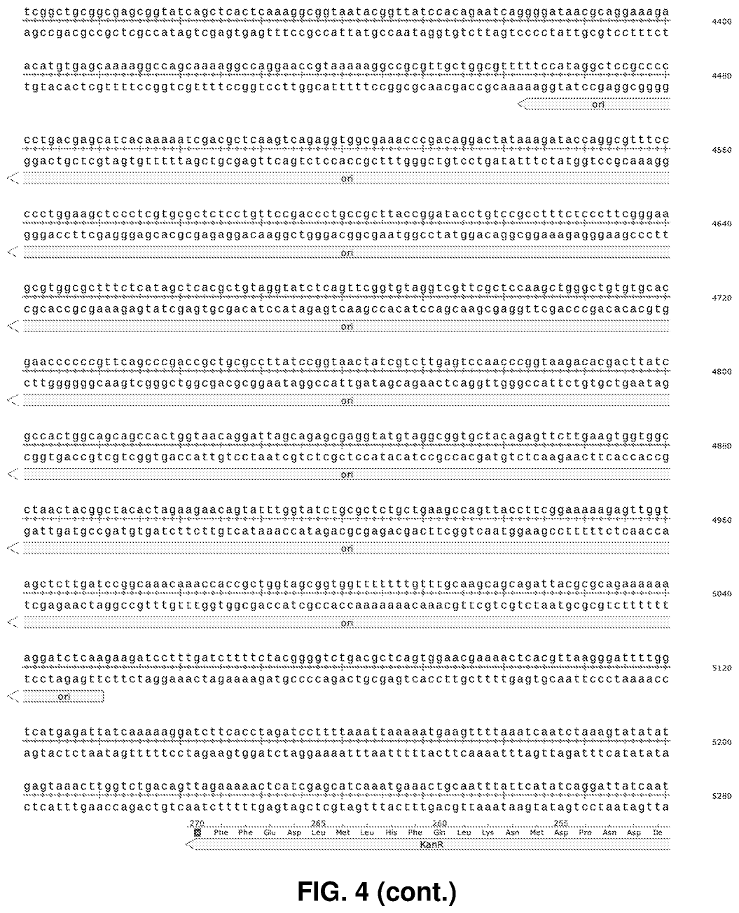

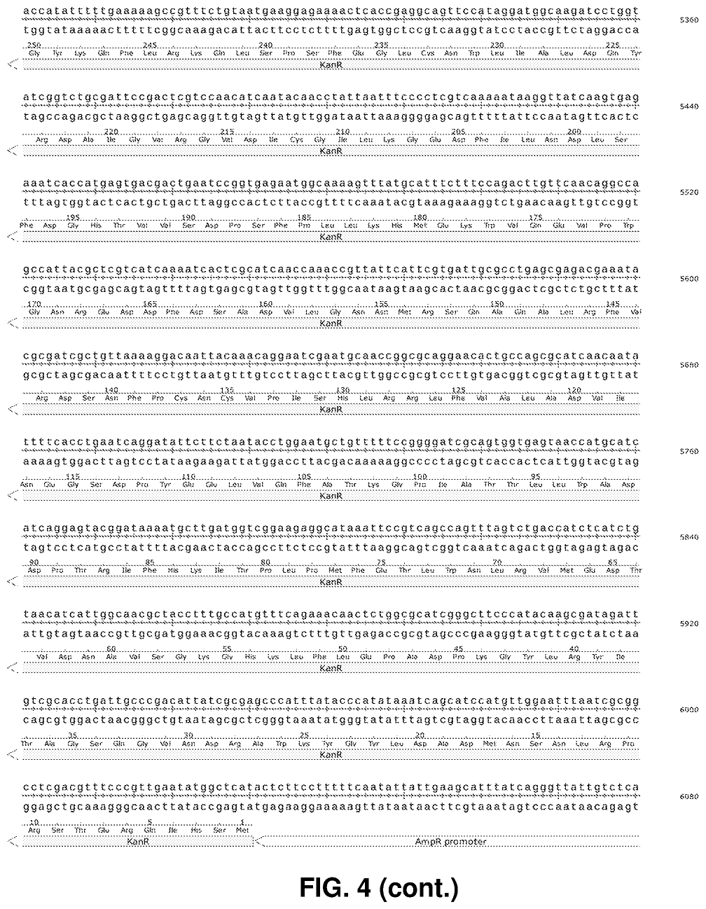

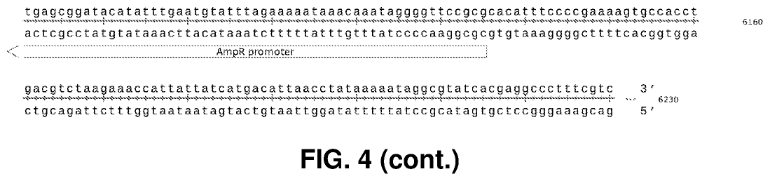

In some embodiments, an AAV vector plasmid is provided that may be used to prepare a recombinant AAV viral particle having a recombinant genome comprising a nucleotide sequence encoding the target gene operably linked to regulatory elements that promote expression in appropriate tissues. The plasmids provided herein generally have an origin of replication and selectable markers to permit reproduction of the plasmid and use in host cells for generating the recombinant AAV viral particles described herein. Exemplary plasmids, and their sequences, as depicted in FIGS. 1-6. The plasmids provided herein include plasmids comprising the expression cassettes described herein. In particular, the AAV vector plasmids may comprise an expression cassette having a nucleotide sequence of SEQ ID NO: 3, 4 or 5. In certain embodiments, the AAV vector plasmid comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 3, SEQ ID NO.: 4, SEQ ID NO.: 5, SEQ ID NO.: 19, SEQ ID NO.: 20 or SEQ ID NO.: 21. An AAV vector plasmid may have a nucleotide sequence of SEQ ID NO.: 6, SEQ ID NO.: 7, or SEQ ID NO.: 8.

In some embodiments, the AAV vector plasmid further comprises a bacterial expressing region. In some embodiments, the bacterial expressing region comprises a bacterial promoter and a nucleic acid that encodes a bacterial selecting region. In some embodiments, the nucleic acid that encodes the bacterial selecting region is operably linked to the bacterial promoter. In some embodiments, the nucleic acid that encodes the bacterial selecting region comprises a nucleic acid that encodes an antibiotic resistance gene or protein. In some embodiments, the antibiotic resistance gene or protein comprises an ampicillin resistance gene (AmpR) or a kanamycin resistance gene sequence (KanR). In some embodiments the bacteria promoter comprises AmpR promoter or a KanR promoter. In some embodiments, the AAV vector plasmid further comprises an origin of replication. In some embodiments, the origin of replication comprises a CMV origin of replication (ori). In some embodiments, the AAV vector plasmid further comprises a eukaryotic expressing region. In some embodiments, the eukaryotic expressing region comprises a eukaryotic promoter and a nucleic acid that encodes a eukaryotic selecting region. In some embodiments, the nucleic acid that encodes a eukaryotic selecting region is operably linked to the eukaryotic promoter. In some embodiments, the eukaryotic promoter comprises nucleic catabolite activator protein (CAP) binding site or a lactose (lac) promoter. In some embodiments, the eukaryotic selecting region comprises a lac operator. In some embodiments the plasmid comprises an M13 reverse primer region.

### AAV Virion

In some embodiments, a recombinant AAV virion is provided. In some embodiments, the AAV virion comprises an AAV capsid protein and the recombinant AAV vector that comprises the nucleotide sequence encoding hGALT operably linked to regulatory elements. In some embodiments, the recombinant AAV vector is a recombinant scAAV vector. In some embodiments, the recombinant AAV or scAAV vector is a single-stranded DNA. In some embodiments, the AAV capsid protein encapsulates the recombinant AAV vector.

In some embodiments, the AAV capsid protein comprises a VP1, a VP2, and a VP3. The capsid preferably has tropism for appropriate cells and tissues, such as, for example, nervous tissue, CNS, liver, etc. In some embodiments, the capsid protein comprises an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AAV-DJ capsid protein, an AAV-DJ/8 capsid protein, an AAV-Rh10 capsid protein, an AAV-retro capsid protein, an AAV-PHP.B capsid protein, an AAV8-PHP.eB capsid protein, or an AAV-PHP.S capsid protein. In certain embodiments, the rAAV particle has an AAV9 capsid protein, for example, having an amino acid sequence of SEQ ID NO:18. Alternatively, the capsid protein has an amino acid sequence that is 99%, 98%, 95%, 90% or 85% identical to the AAV9 capsid and has the tropism and transduction activity of the AAV9 capsid protein.

In some aspects, the isolated nucleic acids and/or rAAVs described herein can be modified and/or selected to enhance the targeting of the isolated rAAVs to a target tissue (e.g., CNS). Non-limiting methods of modifications and/or selections include AAV capsid serotypes (e.g., AAV9), tissue-specific promoters, and/or targeting peptides. In some aspects, the isolated nucleic acids and rAAVs disclosed herein can comprise AAV capsid serotypes with enhanced targeting to CNS tissues (e.g., AAV9). In some aspects, the isolated nucleic acids and rAAVs described herein can comprise tissue-specific promoters. In some aspects, the isolated nucleic acids and rAAVs described herein can comprise AAV capsid serotypes with enhanced targeting to CNS tissues and tissue-specific promoters. While AAV9 targets CNS tissue, the rAAV9 vectors may also transduce other non-CNS tissues and, thus, the transgenes, under the control of a promoter such as the CAG promoter may be expressed both in the CNS and other tissues outside the CNS.

Methods for obtaining recombinant AAVs having a desired capsid protein can be obtained from U.S. Patent Application Publication Number 2003/0138772, for example. Typically the methods involve culturing a host cell which contains a nucleic acid sequence encoding an AAV capsid protein or fragment thereof; a functional rep gene; sufficient helper functions to permit packaging of the recombinant AAV vector into the AAV capsid proteins; and a recombinant AAV vector plasmid comprising the AAV vector. Typically, capsid proteins are structural proteins encoded by the cap gene of an AAV. In some aspects, wherein the capsid protein comprises VP1, VP2, and VP3, said VP1, VP2, and VP3 are transcribed from a single cap gene via alternative splicing. In some aspects, the molecular weights of VP1, VP2 and VP3 are respectively about 87 kDa, about 72 kDa and about 62 kDa. In some aspects, upon translation, capsid proteins form a spherical 60-mer protein shell around the viral genome. In some aspects, capsid proteins protect a viral genome, deliver a genome and/or interact with a host cell. In some aspects, capsid proteins deliver the viral genome to a host in a tissue specific manner.

In some aspects, components to be cultured in the host cell to package a recombinant AAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components. In particular embodiments, provided are host cells comprising the recombinant AAV constructs or plasmids comprising the target gene sequence, a plasmid providing the AAV rep and cap gene sequences, and a construct providing adenoviral helper proteins as needed to produce the recombinant viral particle.

In some aspects, such a stable host cell can contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In some aspects, a selected stable host cell can contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions useful for producing the rAAV described herein can be delivered to the packaging host cell using any appropriate genetic element (vector, e.g. plasmid). The selected genetic element can be delivered by any suitable method, including those described herein. The methods used to construct any of compositions disclosed herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some aspects, recombinant AAVs can be produced using the triple transfection method (described in detail in U.S. Pat. No. 6,001,650). Typically, the recombinant AAVs can be produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation with the cap gene encoding the capsid proteins of desired serotype, for example, encoding the AAV9 capsid. In some aspects, the AAV helper function vector can support efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells. Disclosed herein are transfected host cells. The term “transfection” is used to refer to the uptake of foreign DNA by a cell, and a cell has been “transfected” when exogenous DNA has been introduced through the cell membrane. Examples of methods of transfection include Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

In one aspect, a cell is provided. In some embodiments, the cell comprises an AAV second plasmid and an AAV vector plasmid. In some embodiments, the AAV second plasmid comprises rep and cap. In some embodiments, the cap encodes a VP1, a VP2, and a VP3. In some embodiments, the rep encodes rep78, rep68, rep 52, and rep 40. In some embodiments, the AAV vector plasmid comprises the recombinant AAV vector or the recombinant scAAV vector. In some embodiments, the cap is AAV9 cap. In some embodiments, the AAV vector plasmid comprises the expression cassette of SEQ ID NO.: 6, SEQ ID NO.: 7, or SEQ ID NO.: 8.

In another aspect, a method of producing the AAV virion is provided. In some embodiments, the method comprises transfecting a cell with a second plasmid and at least one of a vector plasmid or the AAV vector construct. In some embodiments, the vector plasmid or AAV vector construct comprises the recombinant AAV vector or the recombinant scAAV vector. In some embodiments, the second plasmid comprises cap and rep. In some embodiments, the cap encodes the VP1, the VP2, and the VP3. In some embodiments, the rep encodes rep78, rep68, rep 52, and rep 40. In some embodiments, the vector plasmid or the AAV vector construct comprises the expression cassette having the nucleotide sequence of SEQ ID NO.: 3, SEQ ID NO.: 4, SEQ ID NO.: 5, SEQ ID NO.: 19, SEQ ID NO.: 20 or SEQ ID NO.: 21 or having the nucleotide sequence of SEQ ID NO.: 6, SEQ ID NO.: 7, or SEQ ID NO.: 8.

### Methods of Treatment

In some embodiments, the AAV virion will transduce the cells, or the cells of the tissue or organ. In some embodiments, upon transduction, the AAV vector will be released from encapsulation from the capsid protein. In some embodiments, the AAV vector will be released into the cytosol or nucleus of the cell. In some embodiments, the cell's transcription machinery will bind to the promoter of the AAV vector. In some embodiments, the cell will express the nucleic acid encoding the protein. In some embodiments, the cell will express the protein. In some embodiments, the cell will express the nucleic acid encoding the GALT. In some embodiments, the cell will express the GALT. In some embodiments, the GALT will undergo its enzymatic activity (i.e. conduct at least the forward reaction or the reverse reaction of the interconversion of uridine diphosphate-glucose and galactose-1-phosphate into glucose-1-phosphate and uridine diphosphate-galactose).

In some embodiments, a method for treating at least one of galactosemia, insufficient galactose metabolism, GALT-deficiency, GALT-insufficiency, or symptoms of insufficient galactose metabolism in a subject in need thereof are provided. In some embodiments, a method of increasing galactose metabolism in a subject is provided. In some embodiments, a method of reducing a disease condition in a subject, who suffers from galactosemia, are provided. In some embodiments, said disease condition comprises jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency. In some embodiments, the method comprises administering to the subject a therapeutically effective amount of the AAV virion.

In some embodiments, a subject has Type 1 galactosemia. In some embodiments, a subject does not express GALT protein or expresses undetectable levels of GALT protein. In some embodiments, a subject lacks GALT enzymatic activity or has undetectable levels of GALT enzymatic activity. In some embodiments, a subject has increased levels of galactose compared to a subject that does not have galactosemia. In some embodiments, a subject has increased levels of GAL-1P compared to a subject that does not have galactosemia. In some embodiments, a subject has increased levels of galactitol compared to a subject that does not have galactosemia. In some embodiments, a subject has lower levels of GALT protein expression compared to a subject that does not have galactosemia. In some embodiments, a subject has reduced levels of GALT enzymatic activity compared to a subject that does not have galactosemia.

In some embodiments, a method of reducing galactose levels in a subject in need thereof is provided. “Reduced levels” include relative to a subject that has not been treated with the hGALT encoding gene therapy, including a subject that has been on a low galactose diet, or relative to levels characteristic of a subject of similar age and weight as determined by a natural history study of galactosemia patients and wherein the reduction that makes a therapeutic difference to the patient by ameliorating one or more symptoms of the disease. Reduced levels may be by 10%, 20%, 30%, 40% 50%, 60%, 70%, 80%, 85%, 90%, 91%, 92%, 93%, 94%, 95%, 96%, 97%, 98% or 99% relative to the reference. Reduced levels may be by about 10% to about 90%, about 20% to about 90%, about 40% to about 90%, about 50% to about 60%, about 70% to about 90%, about 80% to about 90% or about 90% to about 99% relative to the reference.

In some embodiments, levels of galactose are reduced in the liver by about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in the muscle by about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in the brain by about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in RBCs by about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in the ovary by about 30%, about 35%, about 40%, about 45%, about 50%, about 55%, about 60%, about 65%, about 70%, about 75%, about 80%, about 85% or about 90% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in the eye by about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease. In some embodiments, levels of galactose are reduced in the plasma by about 90%, about 91%, about 92%, about 93%, about 94%, about 95%, about 96%, about 97%, about 98% or about 99% compared to either the subject prior to administration or compared to a similarly situated subject identified in a natural history study wherein the reduction that makes a therapeutic difference to the patient by ameliorating one or more symptoms of the disease.

In some embodiments, a method of reducing galactitol levels by administration of a pharmaceutical composition comprising recombinant AAVs as described herein to a subject in need thereof is provided. In some embodiments, a method of reducing GAL-1P levels by administration of a pharmaceutical composition comprising recombinant AAVs as described herein to a subject in need thereof is provided.

In some embodiments, levels of galactose are reduced in liver, muscle, brain and/or blood plasma and wherein the reduction that makes a therapeutic difference to the patient by ameliorating one or more symptoms of the disease. In some embodiments, levels of galactose are reduced in liver, muscle, brain, eye, ovary, red blood cells, and/or blood plasma. In some embodiments, levels of galactitol are reduced in liver, muscle, brain and/or blood plasma. In some embodiments, levels of galactitol are reduced in liver, muscle, brain, eye, ovary, red blood cells, and/or blood plasma. In some embodiments, levels of GAL-1P are reduced in liver, muscle, brain and/or blood plasma and wherein the reduction that makes a therapeutic difference to the patient by ameliorating one or more symptoms of the disease. In some embodiments, levels of GAL-1P are reduced in liver, muscle, brain, eye, ovary, red blood cells and/or blood plasma and wherein the reduction that makes a therapeutic difference to the patient by ameliorating one or more symptoms of the disease. The reduced levels may be achieved within 1 month, 2 months, 3 months, 6 months, 1 year or 2 years after the gene therapy administration.

In some embodiments, levels of galactose are reduced in liver, muscle, brain and/or blood plasma compared to a similarly-situated subject and wherein the reduction that makes a therapeutic difference to the patient by ameliorating one or more symptoms of the disease. In some embodiments, levels of galactitol are reduced in liver, muscle, brain and/or blood plasma compared to a similarly-situated subject who has not been administered the gene therapy therapeutic described herein. In some embodiments, levels of GAL-1P are reduced in liver, muscle, brain and/or blood plasma compared to a similarly-situated subject who has not been administered the gene therapy therapeutic described herein. In some embodiments, levels of galactose are reduced in liver, muscle, brain, eye, ovary and/or blood plasma compared to a similarly-situatued subject and wherein the reduction that makes a therapeutic difference to the patient by ameliorating one or more symptoms of the disease. In some embodiments, levels of galactitol are reduced in liver, muscle, brain, eye, ovary, red blood cell, and/or blood plasma compared to a similarly-situatued subject who has not been administered the gene therapy therapeutic described herein. In some embodiments, levels of GAL-1P are reduced in liver, muscle, brain, eye, ovary and/or blood plasma compared to a similarly-situated subject who has not been administered the gene therapy therapeutic described herein. A “similarly-situated subject” means a subject having similar galactose levels in all tissues or in one or more of liver, muscle, brain, eye, ovary and/or blood plasma (within about 20%, 15%, 10%, 9%, 8%, 7%, 6%, 5%, 4%, 3%, 2%, 1% or less than about 1%) as the subject being treated prior to the gene therapy administration. In some embodiments, a similarly situated subject will have the same mutation leading to reduced or absent GALT protein expression and/or GALT enzyme activity as the subject being treated.

In some embodiments, levels of galactose are reduced in liver, muscle, brain and/or blood plasma compared to similarly-situated subject on a low galactose diet but not treated with the gene therapy therapeutic described herein. In some embodiments, levels of galactitol are reduced in liver, muscle, brain and/or blood plasma compared to a similarly-situated subject on a low galactose diet but not treated with the gene therapy therapeutic described herein. In some embodiments, levels of GAL-1P are reduced in liver, muscle, brain and/or blood plasma compared to a similarly-situated subject on a low galactose diet but not treated with the gene therapy therapeutic described herein.

In some embodiments, levels of galactose are reduced in liver, muscle, brain, eye, ovary and/or blood plasma compared to similarly-situated subject on a low galactose diet but not treated with the gene therapy therapeutic described herein. In some embodiments, levels of galactitol are reduced in liver, muscle, brain, eye, ovary and/or blood plasma compared to a similarly-situated subject on a low galactose diet but not treated with the gene therapy therapeutic described herein. In some embodiments, levels of GAL-1P are reduced in liver, muscle, brain, eye, ovary and/or blood plasma compared to a similarly-situatued subject on a low galactose diet but not treated with the gene therapy therapeutic described herein.

In some embodiments, levels of galactose are reduced in liver, muscle, brain and/or blood plasma compared to the levels in a similarly situated subject as identified in a natural history study. In some embodiments, levels of galactitol are reduced in liver, muscle, brain and/or blood plasma compared to the levels in a similarly situated subject as identified in a natural history study. In some embodiments, levels of GAL-1P are reduced in liver, muscle, brain and/or blood plasma compared to the levels in a similarly situated subject as identified in a natural history study.

In some embodiments, levels of galactose are reduced in liver, muscle, brain, eye, ovary, red blood cell and/or blood plasma compared to the levels in a similarly situated subject as identified in a natural history study. In some embodiments, levels of galactitol are reduced in liver, muscle, brain, eye, ovary, red blood cell and/or blood plasma compared to the levels in a similarly situated subject as identified in a natural history study. In some embodiments, levels of GAL-1P are reduced in liver, muscle, brain, eye, ovary, red blood cell and/or blood plasma compared to the levels in a similarly situated subject as identified in a natural history study.

In some embodiments, levels of Gal-1P are reduced in liver by about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study and wherein the reduction that makes a therapeutic difference to the patient by ameliorating one or more symptoms of the disease. In some embodiments, levels of Gal-1P are reduced in muscle by about 40%, about 65%, about 70%, about 75%, about 80% or about 85%, about 90%, about 95%, about 96%, about 97%, about 98% or about 99% compared to either the subject prior to administration or compared to a similarly situated subject identified in a natural history study. In some embodiments, levels of Gal-1P are reduced in brain by about 30%, about 40%, about 65%, about 70%, about 75%, about 80%, about 85%, about 90%, about 95%, or about 99% compared to either the subject prior to administration or compared to a similarly situated subject identified in a natural history study and wherein the reduction that makes a therapeutic difference to the patient by ameliorating one or more symptoms of the disease. In some embodiments, levels of Gal-1P are reduced in RBCs by about 40%, about 45%, about 50%, about 55%, about 60%, about 65% or about 70% compared to either the subject prior to administration or compared to a similarly situated subject identified in a natural history study and wherein the reduction that makes a therapeutic difference to the patient by ameliorating one or more symptoms of the disease. In some embodiments, levels of Gal-1P are reduced in ovary by about 65%, about 70%, about 75%, about 80%, about 85%, about 90%, about 95%, or about 99% compared to either the subject prior to administration or compared to a similarly situated subject identified in a natural history study and wherein the reduction that makes a therapeutic difference to the patient by ameliorating one or more symptoms of the disease. In some embodiments, levels of Gal-1P are reduced in eye by about 35%, about 40%, about 45% or about 50% compared to either the subject prior to administration or compared to a similarly situated subject identified in a natural history study and wherein the reduction that makes a therapeutic difference to the patient by ameliorating one or more symptoms of the disease.

In some embodiments, levels of galactitol are reduced in liver by about 25%, about 30%, about 35%, about 40%, about 45%, about 50%, about 55%, about 60%, about 65%, about 70%, about 75%, about 80%, about 85% or about 90% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in muscle by about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in brain by about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in plasma by about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in RBCs by about 70%, about 75%, about 80%, about 85%, about 90%, about 91%, about 92%, about 93%, about 94%, about 95%, about 96%, about 97%, about 98% or about 99% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in ovary by about 30%, about 35%, about 40%, about 45%, about 50%, about 55%, about 60%, about 65%, about 70%, about 75% or about 80% compared to either the subject prior to administration or compared to a similarly situated subject identified in a natural history study and wherein the reduction that makes a therapeutic difference to the patient by ameliorating one or more symptoms of the disease. In some embodiments, levels of galactitol are reduced in eye by about 60%, about 65%, about 70%, about 75%, about 80%, about 85%, about 90% or about 95% compared to either the subject prior to administration or compared to a similarly situated subject identified in a natural history study and wherein the reduction that makes a therapeutic difference to the patient by ameliorating one or more symptoms of the disease.

In some embodiments, a method of increasing GALT protein expression in a subject in need thereof is provided. Increased levels may be by about 10%, 20%, 30%, 40% 50%, 60%, 70%, 80% 90%, 100%, 110%, 120%, 130%, 140%, 150%, 160%, 170%, 180%, 190% or 200% relative to the reference. Increased levels may be by about 10% to about 200%, about 20% to about 200%, about 40% to about 200%, about 50% to about 200%, about 70% to about 200%, about 80% to about 200%, about 90% to about 200%, about 100% to about 200%, about 110% to about 200%, about 120% to about 200%, about 130% to about 200%, about 150% to about 200%, about 160% to about 200%, about 170% to about 200% or about 180% to about 200% relative to the reference. In some embodiments, GALT protein expression is increased in liver, muscle, brain, eye and/or blood plasma as compared to the GALT protein expression levels either prior to the gene therapy administration or relative to the levels in a similarly situated subject as determined in a natural history study. In some embodiments, GALT protein expression is increased in brain. In some embodiments, GALT protein expression is increased in cortex and/or cerebral tissues. In some embodiments, GALT protein expression is increased in neuronal cells and/or glial cells. In some embodiments, GALT protein expression is increased in Purkinje neurons. In some embodiments, GALT protein expression is increased in blood plasma. In some embodiments, GALT protein expression is increased in red blood cells. In all cases, the increase is relative to the GALT protein expression levels either prior to the gene therapy administration or relative to the levels in a similarly situated subject as determined in a natural history study and wherein the reduction that makes a therapeutic difference to the patient by ameliorating one or more symptoms of the disease.

In some embodiments, a method of increasing GALT enzyme activity in a subject in need thereof is provided. In some embodiments, GALT enzyme activity is increased in liver, muscle, eye, blood plasma and/or brain. In some embodiments, GALT enzyme activity is increased in brain. In some embodiments, GALT enzyme activity is increased in cortex and/or cerebral tissues. In some embodiments, GALT enzyme activity is increased in neuronal cells and/or glial cells. In some embodiments, GALT enzyme activity is increased in Purkinje neurons. In some embodiments, GALT enzyme activity is increased in liver. In some embodiments, GALT enzyme activity is increased in muscle. In some embodiments, GALT enzyme activity is increased in blood plasma. In some embodiments, GALT enzyme activity is increased in red blood cells. In all cases, the increase is relative to the GALT protein expression levels either prior to the gene therapy administration or relative to the levels in a similarly situated subject as determined in a natural history study and wherein the reduction that makes a therapeutic difference to the patient by ameliorating one or more symptoms of the disease.

In some embodiments, GALT enzyme activity is increased in liver by about 1000%, about 1500%, about 2000%, about 2500%, about 3000%, about 3500%, about 4000%, about 4500%, about 5000%, about 5500%, about 6000%, about 6500%, about 7000%, or about 7500% compared to a WT/WT subject without a GALT enzyme deficiency. In some embodiments, GALT enzyme activity is increased in brain by about 150%, about 200%, about 250%, about 300%, about 350%, about 400%, about 450%, about 500%, or about 550% compared to a WT/WT subject without a GALT enzyme deficiency. In some embodiments, GALT enzyme activity is increased in skeletal muscle by about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500, or about 10,000% compared to a WT/WT subject without a GALT enzyme deficiency. In some embodiments, GALT enzyme activity is increased in eye by about 90%, about 100%, about 130%, about 150%, about 200%, about 250% or about 300% compared to a WT/WT subject without a GALT enzyme deficiency.

In some embodiments, GALT enzyme activity is increased in liver by about 35%, about 40%, about 50%, about 55%, about 60%, about 65%, about 70%, about 75%, about 80%, about 85%, about 90%, about 95%, about 100%, about 105%, about 110%, about 115%, about 120%, about 125%, about 130%, about 140%, about 150%, about 160%, about 170%, about 180%, about 190%, or about 200% compared to a WT/WT subject without a GALT enzyme deficiency. In some embodiments, GALT enzyme activity is increased in brain by about 25%, about 30%, about 35%, about 40%, about 45%, about 50%, about 55%, about 60%, about 65%, about 70%, about 75%, about 80%, about 85%, about 90%, about 95%, or about 100% compared to a WT/WT subject without a GALT enzyme deficiency. In some embodiments, GALT enzyme activity is increased in muscle by about 95%, about 100%, about 105%, about 110%, about 115%, about 120%, about 125%, about 130%, about 140%, about 150%, about 160%, about 170%, about 180%, about 190%, about 200%, about 210%, about 220%, about 230%, about 240% or about 250% compared to a WT/WT subject without a GALT enzyme deficiency.

In some embodiments, a method of improving motor and/or neuromuscular coordination in a subject in need thereof comprising administering to the subject a therapeutically effective amount of an AAV virion of the present disclosure is provided in which the improvement of motor and/or neuromuscular deficit is reduced relative to a similarly situated subject that has not been administered the gene therapy therapeutic. Motor and/or neuromuscular coordination may be assessed according to methods known in the art.

In some embodiments, a method of improving motor strength in a subject in need thereof comprising administering to the subject a therapeutically effective amount of an AAV virion of the present disclosure is provided in which the improvement of motor strength is improved relative to a similarly situated subject that has not been administered the gene therapy therapeutic. Motor strength may be assessed according to methods known in the art.

In some embodiments, a method of improving spatial learning comprising administering to the subject a therapeutically effective amount of an AAV virion of the present disclosure is provided in which the improvement in spatial learning is improved relative to a similarly situated subject that has not been administered the gene therapy therapeutic. Spatial learning may be assessed according to methods known in the art.

In some embodiments, a method of reducing and/or rescuing ovarian failure comprising administering to the subject a therapeutically effective amount of an AAV virion of the present disclosure is provided in which the reduction or rescuing of ovarian failure is relative to a similarly situated subject that has not been administered the gene therapy therapeutic. Ovarian failure may be assessed according to methods known in the art.

In some embodiments, a method of regulating follicle-stimulating hormone, luteinizing hormone and/or anti-Müllerian hormone comprising administering to the subject a therapeutically effective amount of an AAV virion of the present disclosure is provided in which regulation of follicle-stimulating hormone, luteinizing hormone and/or anti-Müllerian hormone is relative to a similarly situated subject that has not been administered the gene therapy therapeutic. Regulating follicle-stimulating hormone, luteinizing hormone and/or anti-Müllerian hormone may be assessed according to methods known in the art.

In some embodiments, a method of inhibiting cataract formation or promoting cataract resorption in a subject in need thereof is provided. In some embodiments, a method of limiting the severity or risk of cataract formation or promoting cataract resorption in a subject in need thereof is provided in which the severity and/or risk of cataract formation is reduced compared to prior to gene therapy administration or reduced relative to a similarly situated subject as identified in a natural history study. In some embodiments, a method of limiting the severity or risk of cataract formation or promoting cataract resorption in a subject in need thereof is provided in which the severity and/or risk of cataract formation is reduced relative to a similarly situated subject that has not been administered the gene therapy therapeutic. In some embodiments, the AAV virion is administered prior to the development of cataracts.

In some embodiments, a method of improving (i.e., increasing the rate of or the amount of) weight gain in a subject in need thereof is provided either relative to the rate of weight gain in the subject prior to the gene therapy administration or relative to the rate or amount of weight gain in a similarly situated subject who has not been administered the gene therapy therapeutic. In some embodiments, a method of reducing a pre-pubertal growth delay in a subject in need thereof is provided.

In some embodiments, a method of reducing the severity of a visceral neuromuscular deficit is provided in which the severity of the visceral neuromuscular deficit is reduced compared to prior to the gene therapy administration or reduced relative to a similarly situated subject as identified in a natural history study. In some embodiments, a method of reducing the severity of a visceral neuromuscular deficit in a subject in need thereof is provided in which the severity and/or risk of visceral neuromuscular deficit is reduced relative to a similarly situated subject that has not been administered the gene therapy therapeutic. Visceral neuromuscular deficit may be assessed according to methods known in the art.

In some embodiments, a method of reducing the severity of neurological and/or socioemotional deficits is provided in which the severity of neurological and/or socioemotional deficits is reduced compared to prior to the gene therapy administration or reduced relative to the levels in a similarly situated subject as identified in a natural history study. In some embodiments, a method of reducing the severity of neurological and/or socioemotional deficits is provided in which the severity is reduced relative to a similarly situated subject that has not been administered the gene therapy therapeutic. Neurological and/or socioemotional deficits include locomotor function/activity, such as tremor and ataxia, anxiety and/or depression. Neurological and/or socioemotional deficits may be assessed according to methods known in the art.

In some embodiments, the AAV virion is administered prior to the onset of puberty. In some embodiments, AAV virion is administered to a young pediatric subject. In some embodiments, a “young pediatric subject” is about 4 years old, about 3.5 years old, about 3 years old, about 2.5 years old, about 2 years old, about 1.5 years old, about 1 year old or about 6 months old.

In some embodiments, the AAV virion is administered to a pediatric subject about 5 years old, about 6 years old, about 7 years old, about 8 years old, about 9 years old, about 10 years old, about 11 years old, about 12 years old, about 13 years old, about 14 years old, about 15 years old, about 16 years old or about 17 years old.

In some embodiments, the methods described herein further comprise the step of monitoring levels of galactose, GAL-1P and/or galactitol. Methods of measuring galactose levels, GAL-1P and/or galactitol are known in the art and include immunohistochemistry, enzymatic assays, mass spectrometry (GC/MS and LC/MS), etc.

In some embodiments, the subject has a GALT, which comprises a mutation of at least one of: Q188R, N314D, L218L, S135L, K285N, L195P, T138M, Y209C, and IVS2-2A>G. In some embodiments, the subject has a deletion of about 5 kb in one or both of the genes encoding GALT, wherein the deletion first identified in individuals of Ashkenazi Jewish ancestry. In some embodiments, the subject has classic galactosemia, clinical variant galactosemia, or Duarte galactosemia.

In some embodiments, the administering or treating comprises: intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation. In some embodiments, the method comprises contacting the AAV virion, or an effective amount thereof, with at least one of the liver, the spleen, a muscle, a kidney, the blood, a lens, an eye, the cerebellum, the brainstem, the basal ganglia, the hypothalamus, the preoptic area, a hippocampus, a striatum, a cortex, a motor cortex, a prefrontal cortex, a somatosensory cortex, a temporal cortex, a visual cortex, an occipital lobe, a temporal lobe, a parietal lobe, a frontal lobe, a bone, a reproductive organ, an ovary, a testis, a skin surface, a prostate, a uterus, or a pancreas of the subject.

In some embodiments, the administration, treating, contacting, or effective amount comprises at least, or no more than, 10⁴, 10⁵, 10⁶, 10⁷, 10⁸, 10⁹, 10¹⁰, 10¹¹, 10¹², 10¹³, 10¹⁴, or 10¹⁵ plague forming units (PFU), PFU/mL, or virions of the AAV.

In some embodiments, the administering or treating can comprise at least, or no more than, 1, 2, 3, 4, 5, 6, 7, 8, 9, or 10 administrations or treatments. In some embodiments, successful treatment and/or repair is determined when one or more of the following is detected: alleviation or amelioration of one or more of symptoms of the treated subject's disease, disorder, or condition, diminishment of extent of the subject's disease, disorder, or condition, stabilized (i.e., not worsening) state of a disease, disorder, or condition, delay or slowing of the progression of the disease, disorder, or condition, and amelioration or palliation of the disease, disorder, or condition. In some embodiments, success of treatment is determined by detecting the presence repaired target polynucleotide in one or more cells, tissues, or organs isolated from the subject. In some embodiments, success of treatment is determined by detecting the presence of polypeptide encoded by the repaired target polynucleotide in one or more cells, tissues, or organs isolated from the subject.

In some embodiments, the recombinant AAV (rAAV) virion can be administered in a composition at a concentration of at least, or no more than, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% or 1.0% weight by weight, weight by volume, or mole by mole.

In some embodiments, the composition optionally comprises a suitable carrier. Suitable carriers can be selected for the indication for which the rAAV is directed. For example, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also include, in addition to the rAAV virion and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In particular embodiments, the rAAV virion is administered in a pharmaceutical composition comprising phosphate buffered saline (PBS), pH 7.3 and 0.001% of a pharmaceutically acceptable non-ionic surfactant, such as, for example, pluronic F-68 (PF68), or other appropriate pharmaceutically acceptable buffers or excipients. The formulation may be frozen until ready for use and then thawed and administered. In some embodiments, the pharmaceutical composition can comprise 10 mM Tris, 150 mM NaCl, 0.02% poloxamer 188, 1 mM MgCl2, adjusted to a pH of 8.0. In some embodiments, the composition can further comprise sugar, sorbitol, or trehalose. In some embodiments, the sugar is at least, or no more than, 0.0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% weight by weight, weight by volume, or mole by mole. In some embodiments, the sugars is sucrose, glucose, or lactose. In some embodiments, the sorbitol is at least, or no more than, 0.0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% weight by weight, weight by volume, or mole by mole. In some embodiments, the trehalose is at least, or no more than, 0.0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% weight by weight, weight by volume, or mole by mole.

In some aspects, the compositions disclosed herein can comprise an rAAV virion alone, or in combination with one or more other viruses (e.g., a second rAAV virion encoding having one or more different transgenes). In some aspects, a composition can comprise 1, 2, 3, 4, 5, 6, 7, 8, 9, 10, or more different rAAVs virion each having one or more different transgenes.

Recombinant AAV virions can be administered in sufficient amounts to transfect the cells of a desired tissue and to provide sufficient levels of gene transfer and expression without undue adverse effects. In some aspects, acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. In some aspects, the route of administration can be by intracerebroventricular injection. Routes of administration may be combined, if desired.

The dose of rAAV virions required to achieve a particular “therapeutic effect,” e.g., the units of dose in genome copies/per kilogram of body weight (GC/kg) or vector genomes (vg) per kilogram of body weight (kg), the units of dose in genome copies per brain volume, and units of dose in genome copies per CSF volume, will vary based on several factors including, but not limited to: the route of rAAV virion administration, the level of gene or RNA expression required to achieve a therapeutic effect, the specific disease or disorder being treated, and the stability of the gene or RNA product.

In some embodiments, an effective amount of an rAAV virion can be an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV virion can be in the range from about 1 ml to about 100 ml of solution containing from about 10⁶ to 10¹⁶ genome copies (e.g., from 1×10⁶ to 1×10¹⁶, inclusive). In methods disclosed herein, the therapeutically effective dose is between 6×10¹³ vg/kg to 6×10¹⁴ vg/kg, including 7×10¹³ vg/kg, 8×10¹³ vg/kg, 9×10¹³ vg/kg, 1×10¹⁴ vg/kg, 2×10¹⁴ vg/kg, 3×10¹⁴ vg/kg, 4×10¹⁴ vg/kg, or 5×10¹⁴ vg/kg (or alternatively, genome copies per brain volume, CSF volume or other measurement appropriate for ICV or ICM delivery). In some aspects, a dosage between about 10¹¹ to 10¹² per kg or appropriate measurement rAAV genome copies can be appropriate. In some aspects, a dosage of between about 10¹¹ to 10¹³ per kg or appropriate measurement rAAV genome copies can be appropriate. In some aspects, a dosage of between about 10¹¹ to 10¹⁴ per kg or appropriate measurement rAAV genome copies can be appropriate. In some aspects, a dosage of between about 10¹¹ to 10¹⁵ per kg or appropriate measurement rAAV genome copies can be appropriate. In some aspects, a dosage of about 1×10¹⁴ vector genome (vg) copies per kg or appropriate measurement can be appropriate. In some aspects, the dosage can vary or be reduced when specifically targeting one or more brain region(s). In some aspects, a dosage between about 10⁷ to 10⁸ rAAV genome copies per kg or appropriate measurement can be appropriate. In some aspects, a dosage of between about 10⁸ to 10⁹ rAAV genome copies per kg or appropriate measurement can be appropriate. In some aspects, a dosage of between about 10⁹ to 10¹⁰ rAAV genome copies per kg or appropriate measurement can be appropriate. In some aspects, a dosage of between about 10¹⁰ to 10¹¹ rAAV genome copies per kg or other appropriate measurement can be appropriate.

In some aspects, a potential side-effect for administering an AAV virion to a subject can be an immune response in the subject to the AAV virion, including inflammation, and, and may depend on the route of administration, and in particularly, when the administration of an AAV virion is systemic. In some aspects, a subject can be immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, an interleukin-6 inhibitor, an anti-interleukin-6 antibody, an interleukin-6 receptor inhibitor, an anti-interleukin-6 receptor antibody, and any combination thereof.

In some aspects, methods disclosed herein can further comprise the step of inducing immunosuppression (e.g., administering one or more immunosuppressive agents) in a subject prior to the subject being administered an rAAV virion (e.g., an rAAV virion or pharmaceutical composition as disclosed herein). In some aspects, a subject can be immunosuppressed (e.g., immunosuppression is induced in the subject) between about 30 days and about 0 days (e.g., any time between 30 days until administration of the rAAV virion, inclusive) prior to administration of the rAAV virion to the subject. In some aspects, the subject can be pre-treated with immune suppression agent (e.g., rituximab, sirolimus, and/or prednisone) for at least 7 days.

In some aspects, immunosuppression of a subject maintained during and/or after administration of a rAAV virion or pharmaceutical composition. In some aspects, a subject can be immunosuppressed (e.g., administered one or more immunosuppressants) for between 1 day and 1 year after administration of the rAAV virion or pharmaceutical composition.

In some aspects, rAAV virion compositions can be formulated to reduce aggregation of AAV virions in the composition, particularly where high rAAV virion concentrations are present (e.g., 10¹³ GC/ml or more). Methods for reducing aggregation of rAAVs include, for example, addition of surfactants, pH adjustment, salt concentration adjustment, etc. (See, e.g., Wright F R, et al., Molecular Therapy (2005) 12, 171-178.)

In some aspects, these formulations can contain at least about 0.1% of the active compound or more, although the percentage of the active ingredient(s) may, of course, be varied and can be conveniently be between about 1 or 2% and about 70% or 80% or more of the weight or volume of the total formulation. Naturally, the amount of active compound in each therapeutically-useful composition can be prepared in such a way that a suitable dosage can be obtained in any given unit dose of the compound. Factors such as solubility, bioavailability, biological half-life, route of administration, product shelf life, as well as other pharmacological considerations can be contemplated by one skilled in the art of preparing such pharmaceutical formulations, and as such, a variety of dosages and treatment regimens can be desirable.

In some aspects, it will be desirable to deliver the rAAV virions in suitably formulated pharmaceutical compositions as disclosed herein either subcutaneously, intrapancreatically, intranasally, parenterally, intravenously, intramuscularly, intrathecally, or orally, intraperitoneally, intracerebroventricularly, or by inhalation. In some aspects, the administration modalities as described in U.S. Pat. Nos. 5,543,158; 5,641,515 and 5,399,363 can be used to deliver rAAVs. In some embodiments, a preferred mode of administration can be by intracerebroventricular or intrathecal injection.

The pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form can be sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride can be included. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions can be suitable for intravenous, intramuscular, subcutaneous, intracerebroventricular, and intraperitoneal administration. In this connection, a sterile aqueous medium can be employed. For example, one dosage can be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions can be prepared by incorporating the active rAAV virion in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes can be formulated for delivery either encapsulated in a lipid particle, a liposome, a vesicle, a nanosphere, or a nanoparticle or the like.

Such formulations can be used for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-lives (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes can be formed from phospholipids that can be dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core.

Alternatively, nanocapsule formulations of the rAAV virions can be used. Nanocapsules can generally entrap substances in a stable and reproducible way. To avoid side effects due to intracellular polymeric overloading, such ultrafine particles (sized around 0.1 p.m) should be designed using polymers able to be degraded in vivo. Biodegradable polyalkyl-cyanoacrylate nanoparticles that meet these requirements are contemplated for use.

In addition to the methods of delivery described above, the following techniques can also be used as alternative methods of delivering the rAAV compositions to a host.

Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some aspects, the methods can include administering one or more additional therapeutic agents to a subject who has been administered an rAAV or pharmaceutical composition as described herein.

In some aspects, administering the rAAV virions described to a subject promotes expression of GALT by 10-fold compared to a control. In some aspects, administering the rAAV virions described herein to a subject promotes expression of GALT by 5-fold to 100-fold compared to control (e.g., 5-fold to 10-fold, 10-fold to 15-fold, 10-fold to 20-fold, 15-fold to 25-fold, 20-fold to 30-fold, 25-fold to 35-fold, 30-fold to 40-fold, 35-fold to 45-fold, 40-fold to 60-fold, 50-fold to 75-fold, 60-fold to 80-fold, 75-fold to 100-fold compared to a control).

In some aspects, administering the rAAV virions described herein to a subject promotes expression of GALT in a subject (e.g., promotes expression of GALT in the CNS of a subject) by between a 5% and 200% increase (e.g., 5-50%, 25-75%, 50-100%, 75-125%, 100-200%, or 100-150% etc.) compared to a control subject.

As used herein, the term “treating” refers to the application or administration of a composition (e.g., an isolated nucleic acid or rAAV as described herein) to a subject who has a disease or disorder associated with low levels of GALT expression (e.g., GALT deficiency), with the purpose to cure, heal, alleviate, relieve, alter, remedy, ameliorate, improve, or affect the disorder, the symptom of the disease, or the predisposition toward a disease.

Alleviating a disease associated with low levels of GALT expression (e.g., GALT deficiency) includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In particular, administration of the rAAV virion described herein to a human subject suffering from GALT deficiency will within 10 weeks, 15 weeks, 20 weeks, 25 weeks, 30 weeks, 40 weeks, 50 weeks or 1 year after the administration will result in reduction in one or more biomarkers or hallmarks of the disease.

“Development” or “progression” of a disease means initial manifestations and/or ensuing progression of the disease. Development of the disease can be detectable and assessed using standard clinical techniques as well known in the art. However, development also refers to progression that can be undetectable. As used herein the terms development or progression refer to the biological course of the symptoms. “Development” includes occurrence, recurrence, and onset. As used herein “onset” or “occurrence” of a disease can be associated with low levels of GALT expression (e.g., GALT deficiency).

In some aspects, a subject has or is suspected of having a disease or disorder associated with low levels of GALT expression (e.g., GALT deficiency). In some aspects, a subject having a disease or disorder associated with low levels of GALT expression (e.g., GALT deficiency) comprises at least one GALT allele having a loss-of-function mutation (e.g., associated with GALT deficiency). In some aspects, a GALT allele having a loss-of-function mutation (e.g., associated with GALT deficiency) comprises a frameshift mutation, a splice site mutation, a missense mutation, a truncation mutation or a nonsense mutation. A subject may have two GALT alleles having the same loss-of-function mutations (homozygous state) or two GALT alleles having different loss-of-function mutations (compound heterozygous state). In certain aspects, the subject is a carrier of an GALT deficiency and, in certain aspects, is heterozygous for a loss of function allele described herein.

In some aspects, the rAAV virions disclosed herein can be administered in sufficient amounts to transduce the cells of a desired tissue and to provide sufficient levels of gene transfer and expression without undue adverse effects. Pharmaceutically acceptable routes of administration include, but are not limited to, direct delivery to the selected organ (e.g., to the central nervous system), by ICV or administration to the cisterna magna, oral, inhalation (including intranasal and intratracheal delivery), intraocular, intracerebroventricular, intravenous, intramuscular, subcutaneous, intradermal, and other parental routes of administration. Routes of administration can be combined, if desired.

### Kits

Disclosed herein are kits comprising any of the agents described herein. In some aspects, any of the agents disclosed herein can be assembled into pharmaceutical or diagnostic or research kits to facilitate their use in therapeutic, diagnostic or research applications. A kit can include one or more containers housing the components of the disclosure and instructions for use. Specifically, such kits may include one or more agents described herein, along with instructions describing the intended application and the proper use of these agents. In some aspects, the agents in a kit can be in a pharmaceutical formulation and dosage suitable for a particular application and for a method of administration of the agents. Kits for research purposes can contain the components in appropriate concentrations or quantities for running various experiments.

Also disclosed herein are kits for producing a rAAV virions. In some aspects, the kit can comprise a container housing an isolated nucleic acid encoding a GALT1 protein or a portion thereof. In some aspects, the kits can further comprise instructions for producing the rAAV virion. In some aspects, the kit further comprises at least one container housing a recombinant AAV vector, wherein the recombinant AAV vector comprises a transgene (i.e., GALT).

In some aspects, the kits can comprise a container housing a recombinant AAV virion as described supra. In some aspects, the kits can further comprises a container housing a pharmaceutically acceptable carrier. For example, a kit can comprise one container housing a rAAV virion and a second container housing a buffer suitable for injection of the rAAV virion into a subject. In some aspects, the container can be a syringe.

In some aspects, the kits can be designed to facilitate use of the methods described herein by researchers and can take many forms. Each of the compositions of the kit, where applicable, may be provided in liquid form (e.g., in solution), or in solid form, (e.g., a dry powder). In some aspects, some of the compositions can be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions can be associated with the kit, for example, audiovisual (e.g., videotape, DVD, etc.), internet, and/or web-based communications, etc. The written instructions can be in a form prescribed by a governmental agency regulating the manufacture, use or sale of pharmaceuticals or biological products, which instructions can also reflect approval by the agency of manufacture, use or sale for animal administration.

The kits disclosed herein can also contain any one or more of the components described herein in one or more containers. In some aspects, the kits can include instructions for mixing one or more components of the kit and/or isolating and mixing a sample and applying to a subject. The kits can include a container housing agents described herein. The agents can be in the form of a liquid, gel or solid (powder). The agents can be prepared sterilely, packaged in syringe and shipped refrigerated. Alternatively, it can be housed in a vial or other container for storage. A second container can have other agents prepared sterilely. Alternatively, the kits can include the active agents premixed and shipped in a syringe, vial, tube, or other container. The kits can have one or more or all of the components required to administer the agents to an animal, such as a syringe, topical application devices, or iv needle tubing and bag, particularly in the case of the kits for producing specific somatic animal models.

In some aspects, the method disclosed herein can involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some aspects, RNA from the transfected cells can provide a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells can also be infected with a helper virus, such as an Adenovirus or a Herpes Virus. In some aspects, the helper functions can be provided by an adenovirus. The adenovirus can be a wild-type adenovirus, and can be of human or non-human origin, for example, non-human primate (NHP) origin. Similarly, adenoviruses known to infect non-human animals (e.g., chimpanzees, mouse) can also be employed in the methods of the disclosure (See, e.g., U.S. Pat. No. 6,083,716). In addition to wild-type adenoviruses, recombinant viruses or non-viral vectors (e.g., plasmids, episomes, etc.) carrying the necessary helper functions can be utilized. Such recombinant viruses are known in the art and may be prepared according to published techniques. See, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells can also be transfected with a vector (e.g., helper vector) which provides helper functions to the AAV. The vector providing helper functions can provide adenovirus functions, including, e.g., E1a, E1b, E2a, E4ORF6. The sequences of adenovirus gene providing these functions can be obtained from any known adenovirus serotype, such as serotypes 2, 3, 4, 7, 12 and 40, and further including any of the presently identified human types known in the art. Thus, in some aspects, the methods involve transfecting the cell with a vector expressing one or more genes necessary for AAV replication, AAV gene transcription, and/or AAV packaging.

In some aspects, an isolated capsid gene can be used to construct and package recombinant AAV vectors, using methods well known in the art, to determine functional characteristics associated with the novel capsid protein encoded by the gene. For example, isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the isolated capsid gene can be determined by examining the expression of the reporter gene in various tissues (e.g., heart, liver, kidneys) of the animal. Other methods for characterizing isolated capsid genes are disclosed herein and still others are well known in the art.

The kits disclosed can have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits can also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit can involve methods for detecting a latent AAV in a cell. In addition, kits of the disclosure can include instructions, a negative and/or positive control, containers, diluents and buffers for the sample, sample preparation tubes and a printed or electronic table of reference AAV sequence for sequence comparisons.

TABLE 1
Sequences

| | SEQ | |
|---|---|---|
| | ID | |
| Name | NO: | Sequence |
| hGALT | 1 | MSRSGTDPQQRQQASEADAAAATFRANDHQHIRYNPLQDEWVLVSAHRMKRPWQGQVE |
| protein | | PQLLKTVPRHDPLNPLCPGAIRANGEVNPQYDSTFLFDNDFPALQPDAPSPGPSDHPL |
| | | FQAKSARGVCKVMCFHPWSDVTLPLMSVPEIRAVVDAWASVTEELGAQYPWVQIFENK |
| | | GAMMGCSNPHPHCQVWASSFLPDIAQREERSQQAYKSQHGEPLLMEYSRQELLRKERL |
| | | VLTSEHWLVLVPFWATWPYQTLLLPRRHVRRLPELTPAERDDLASIMKKLLTKYDNLF |
| | | ETSFPYSMGWHGAPTGSEAGANWNHWQLHAHYYPPLLRSATVRKFMVGYEMLAQAQRD |
| | | LTPEQAAERLRALPEVHYHLGQKDRETATIA |
| Wild type | 2 | atgagcagaagcggcaccgaccctcagcagagacagcaggcctctgaagccgatgccg |
| hGALT | | ccgctgccaccttcagagccaatgaccaccagcacatccggtacaaccccctgcagga |
| coding | | cgagtgggtgctggtgtccgcccacagaatgaagaggccttggcagggccaggtggaa |
| sequence | | ccccagctgctgaaaaccgtgcccagacacgaccccctgaaccctctgtgtcctggcg |
| | | ccattagagccaacggcgaagtgaacccccagtacgacagcaccttcctgttcgacaa |
| | | cgacttccccgccctgcagcctgatgccccatctcctggacctagcgaccaccctctg |
| | | ttccaggccaagtctgccagaggcgtgtgcaaagtgatgtgcttccacccttggagcg |
| | | acgtgaccctgcccctgatgagcgtgccagagatcagagccgtggtggatgcctgggc |
| | | cagcgtgacagaagaactgggagcccagtacccctgggtgcagatcttcgagaacaag |
| | | ggcgccatgatgggctgcagcaacccccaccctcactgtcaagtgtgggccagcagct |
| | | tcctgcccgatatcgcccagcgggaagagagaagccagcaggcttacaagagccagca |
| | | cggcgagcccctgctgatggaatactccagacaggaactgctgcggaaagaacggctg |
| | | gtgctgaccagcgagcactggctggtgctggtgcctttttgggccacatggccctacc |
| | | agaccctgctgctgcctagaaggcacgtgcggagactgcctgagctgacacccgccga |
| | | gagagatgacctggccagcatcatgaagaaactgctgaccaaatacgacaacctgttc |
| | | gagaccagcttcccctacagcatgggctggcacggcgctcctacaggatctgaggctg |
| | | gcgccaactggaaccactggcagctgcacgcccactactaccccccactgctgagatc |
| | | tgccaccgtgcggaagttcatggtgggatacgagatgctggctcaggcccagagagat |
| | | ctgacccctgaacaggccgccgaacggctgagagcactgcccgaagtgcactaccacc |
| | | tgggacagaaggacagagagacagccacaatcgcctga |
| pscAAV- | 3 | ttggccactccctctctgcgcgctcgctcgctcactgaggccgcccgggcaaagcccg |
| CAG- | | ggcgtcgggcgacctttggtcgcccggcctcagtgagcgagcgagcgcgcagagaggg |
| hGALT | | agtggccaactccatcactaggggttcctgatgtaatacgactcactagttcctggag |
| expression | | gggtggagtcgtgacctaggccattgacgtcaataatgacgtatgttcccatagtaac |
| cassette | | gccaatagggactttccattgacgtcaatgggtggagtatttacggtaaactgcccac |
| (including | | ttggcagtacatcaagtgtatcatatgccaagtacgccccctattgacgtcaatgacg |
| flanking | | gtaaatggcccgcctggcattatgcccagtacatgaccttatgggactttcctacttg |
| ITRs) | | gcagtacatctacgtattagtcatcgctattaccatggtcgaggtgagccccacgttc |
| | | tgcttcactctccccatctcccccccctccccacccccaattttgtatttatttattt |
| | | tttaattattttgtgcagcgatgggggcggggggggggggggggcgcgcgccaggcgg |
| | | ggcggggcggggcgaggggcggggcggggcgaggcggagaggtgcggcggcagccaat |
| | | cagagcggcgcgctccgaaagtttccttttatggcgaggcggcggcggcggcggccct |
| | | ataaaaagcgaagcgcgcggcgggcgggagtcgctgcgcgctgccttcgccccgtgcc |
| | | ccgctccgccgccgcctcgcgccgcccgccccggctctgactgaccgcgttactccca |
| | | caggtgagcgggcgggacggcccttctcctccgggctgtaattagcgcttgggaattc |
| | | gccaccatgagcagaagcggcaccgaccctcagcagagacagcaggcctctgaagccg |
| | | atgccgccgctgccaccttcagagccaatgaccaccagcacatccggtacaaccccct |
| | | gcaggacgagtgggtgctggtgtccgcccacagaatgaagaggccttggcagggccag |
| | | gtggaaccccagctgctgaaaaccgtgcccagacacgaccccctgaaccctctgtgtc |
| | | ctggcgccattagagccaacggcgaagtgaacccccagtacgacagcaccttcctgtt |
| | | cgacaacgacttccccgccctgcagcctgatgccccatctcctggacctagcgaccac |
| | | cctctgttccaggccaagtctgccagaggcgtgtgcaaagtgatgtgcttccaccctt |
| | | ggagcgacgtgaccctgcccctgatgagcgtgccagagatcagagccgtggtggatgc |
| | | ctgggccagcgtgacagaagaactgggagcccagtacccctgggtgcagatcttcgag |
| | | aacaagggcgccatgatgggctgcagcaacccccaccctcactgtcaagtgtgggcca |
| | | gcagcttcctgcccgatatcgcccagcgggaagagagaagccagcaggcttacaagag |
| | | ccagcacggcgagcccctgctgatggaatactccagacaggaactgctgcggaaagaa |
| | | cggctggtgctgaccagcgagcactggctggtgctggtgcctttttgggccacatggc |
| | | cctaccagaccctgctgctgcctagaaggcacgtgcggagactgcctgagctgacacc |
| | | cgccgagagagatgacctggccagcatcatgaagaaactgctgaccaaatacgacaac |
| | | ctgttcgagaccagcttcccctacagcatgggctggcacggcgctcctacaggatctg |
| | | aggctggcgccaactggaaccactggcagctgcacgcccactactaccccccactgct |
| | | gagatctgccaccgtgcggaagttcatggtgggatacgagatgctggctcaggcccag |
| | | agagatctgacccctgaacaggccgccgaacggctgagagcactgcccgaagtgcact |
| | | accacctgggacagaaggacagagagacagccacaatcgcctgaagcgctgctcgaga |
| | | gatctacaacttgtttattgcagcttataatggttacaaataaagcaatagcatcaca |
| | | aatttcacaaataaagcatttttttcactgcattctagttgtggtttgtccaaactca |
| | | tcaatgtatcttaaggcctaggtgagctctggtaccctctccactccctctctgcgcg |
| | | ctcgctcgctcactgaggccgggcgaccaaaggtcgcccgacgcccgggctttgcccg |
| | | ggcggcctcagtgagcgagcgagcgcgcag |
| pAAV2IT | 4 | ttggccactccctctctgcgcgctcgctcgctcactgaggccgcccgggcaaagcccg |
| R-CAG- | | ggcgtcgggcgacctttggtcgcccggcctcagtgagcgagcgagcgcgcagagaggg |
| hGALT | | agtggccaactccatcactaggggttcctgatgtaatacgactcactagttattaata |
| expression | | gtaatcaattacggggtcattagttcatagcccatatatggagttccgcgttacataa |
| cassette | | cttacggtaaatggcccgcctggctgaccgcccaacgacccccgcccattgacgtcaa |
| (including | | taatgacgtatgttcccatagtaacgccaatagggactttccattgacgtcaatgggt |
| flanking | | ggactatttacggtaaactgcccacttggcagtacatcaagtgtatcatatgccaagt |
| ITRs) | | acgccccctattgacgtcaatgacggtaaatggcccgcctggcattatgcccagtaca |
| | | tgaccttatgggactttcctacttggcagtacatctacgtattagtcatcgctattac |
| | | catgggtcgaggtgagccccacgttctgcttcactctccccatctcccccccctcccc |
| | | acccccaattttgtatttatttattttttaattattttgtgcagcgatgggggcgggg |
| | | ggggggggggcgcgcgccaggcggggcggggcggggcgaggggcggggcggggcgagg |
| | | cggagaggtgcggcggcagccaatcagagcggcgcgctccgaaagtttccttttatgg |
| | | cgaggcggcggcggcggcggccctataaaaagcgaagcgcgcggcgggcgggagtcgc |
| | | tgcgttgccttcgccccgtgccccgctccgcgccgcctcgcgccgcccgccccggctc |
| | | tgactgaccgcgttactcccacaggtgagcgggcgggacggcccttctcctccgggct |
| | | gtaattagcgcttggtttaatgacggctcgtttcttttctgtggctgcgtgaaagcct |
| | | taaagggctccgggagggccctttgtgcgggggggagcggctcggggggtgcgtgcgt |
| | | gtgtgtgtgcgtggggagcgccgcgtgcggcccgcgctgcccggcggctgtgagcgct |
| | | gcgggcgcggcgcggggctttgtgcgctccgcgtgtgcgcgaggggagcgcggccggg |
| | | ggcggtgccccgcggtgcgggggggctgcgaggggaacaaaggctgcgtgcggggtgt |
| | | gtgcgtgggggggtgagcagggggtgtgggcgcggcggtcgggctgtaacccccccct |
| | | gcacccccctccccgagttgctgagcacggcccggcttcgggtgcggggctccgtgcg |
| | | gggcgtggcgcggggctcgccgtgccgggcggggggtggcggcaggtgggggtgccgg |
| | | gcggggcggggccgcctcgggccggggagggctcgggggaggggcgcggcggccccgg |
| | | agcgccggcggctgtcgaggcgcggcgagccgcagccattgccttttatggtaatcgt |
| | | gcgagagggcgcagggacttcctttgtcccaaatctggcggagccgaaatctgggagg |
| | | cgccgccgcaccccctctagcgggcgcgggcgaagcggtgcggcgccggcaggaagga |
| | | aatgggcggggagggccttcgtgcgtcgccgcgccgccgtccccttctccatctccag |
| | | cctcggggctgccgcagggggacggctgccttcgggggggacggggcagggcggggtt |
| | | cggcttctggcgtgtgaccggcggctctagagcctctgctaaccatgttcatgccttc |
| | | ttctttttcctacaggccaccatgtcgcgcagtggaaccgatcctcagcaacgccagc |
| | | aggcgtcagaggcggacgccgcagcagcaaccttccgggcaaacgaccatcagcatat |
| | | ccgctacaacccgctgcaggatgagtgggtgctggtgtcagctcaccgcatgaagcgg |
| | | ccctggcagggtcaagtggagccccagcttctgaagacagtgccccgccatgaccctc |
| | | tcaaccctctgtgtcctggggccatccgagccaacggagaggtgaatccccagtacga |
| | | tagcaccttcctgtttgacaacgacttcccagctctgcagcctgatgcccccagtcca |
| | | ggacccagtgatcatccccttttccaagcaaagtctgctcgaggagtctgtaaggtca |
| | | tgtgcttccacccctggtcggatgtaacgctgccactcatgtcggtccctgagatccg |
| | | ggctgttgttgatgcatgggcctcagtcacagaggagctgggtgcccagtacccttgg |
| | | gtgcagatctttgaaaacaaaggtgccatgatgggctgttctaacccccacccccact |
| | | gccaggtatgggccagcagtttcctgccagatattgcccagcgtgaggagcgatctca |
| | | gcaggcctataagagtcagcatggagagcccctgctaatggagtacagccgccaggag |
| | | ctactcaggaaggaacgtctggtcctaaccagtgagcactggttagtactggtcccct |
| | | tctgggcaacatggccctaccagacactgctgctgccccgtcggcatgtgcggcggct |
| | | acctgagctgacccctgctgagcgtgatgatctagcctccatcatgaagaagctcttg |
| | | accaagtatgacaacctctttgagacgtcctttccctactccatgggctggcatgggg |
| | | ctcccacaggatcagaggctggggccaactggaaccattggcagctgcacgctcatta |
| | | ctaccctccgctcctgcgctctgccactgtccggaaattcatggttggctacgaaatg |
| | | cttgctcaggctcagagggacctcacccctgagcaggctgcagagagactaagggcac |
| | | ttcctgaggttcattaccacctggggcagaaggacagggagacagcaaccatcgcctg |
| | | a |
| pAAV2IT | 5 | ttggccactccctctctgcgcgctcgctcgctcactgaggccgcccgggcaaagcccg |
| R-EF1a- | | ggcgtcgggcgacctttggtcgcccggcctcagtgagcgagcgagcgcgcagagaggg |
| hGALT | | agtggccaactccatcactaggggttcctgatgtaatacgactcactagtgctccggt |
| expression | | gcccgtcagtgggcagagcgcacatcgcccacagtccccgagaagttggggggagggg |
| cassette | | tcggcaattgaaccggtgcctagagaaggtggcgcggggtaaactgggaaagtgatgt |
| (including | | cgtgtactggctccgcctttttcccgagggtgggggagaaccgtatataagtgcagta |
| flanking | | gtcgccgtgaacgttctttttcgcaacgggtttgccgccagaacacaggtaagtgccg |
| ITRs) | | tgtgtggttcccgcgggcctggcctctttacgggttatggcccttgcgtgccttgaat |
| | | tacttccacgcccctggctgcagtacgtgattcttgatcccgagcttcgggttggaag |
| | | tgggtgggagagttcgaggccttgcgcttaaggagccccttcgcctcgtgcttgagtt |
| | | gaggcctggcctgggcgctggggccgccgcgtgcgaatctggtggcaccttcgcgcct |
| | | gtctcgctgctttcgataagtctctagccatttaaaatttttgatgacctgctgcgac |
| | | gctttttttctggcaagatagtcttgtaaatgcgggccaagatctgcacactggtatt |
| | | tcggtttttggggccgcgggcggcgacggggcccgtgcgtcccagcgcacatgttcgg |
| | | cgaggcggggcctgcgagcgcggccaccgagaatcggacgggggtagtctcaagctgg |
| | | ccggcctgctctggtgcctggcctcgcgccgccgtgtatcgccccgccctgggcggca |
| | | aggctggcccggtcggcaccagttgcgtgagcggaaagatggccgcttcccggccctg |
| | | ctgcagggagctcaaaatggaggacgcggcgctcgggagagcgggcgggtgagtcacc |
| | | cacacaaaggaaaagggcctttccgtcctcagccgtcgcttcatgtgactccacggag |
| | | taccgggcgccgtccaggcacctcgattagttctcgagcttttggagtacgtcgtctt |
| | | taggttggggggaggggttttatgcgatggagtttccccacactgagtgggtggagac |
| | | tgaagttaggccagcttggcacttgatgtaattctccttggaatttgccctttttgag |
| | | tttggatcttggttcattctcaagcctcagacagtggttcaaagtttttttcttccat |
| | | ttcaggtgtcgtgagccaccatgtcgcgcagtggaaccgatcctcagcaacgccagca |
| | | ggcgtcagaggcggacgccgcagcagcaaccttccgggcaaacgaccatcagcatatc |
| | | cgctacaacccgctgcaggatgagtgggtgctggtgtcagctcaccgcatgaagcggc |
| | | cctggcagggtcaagtggagccccagcttctgaagacagtgccccgccatgaccctct |
| | | caaccctctgtgtcctggggccatccgagccaacggagaggtgaatccccagtacgat |
| | | agcaccttcctgtttgacaacgacttcccagctctgcagcctgatgcccccagtccag |
| | | gacccagtgatcatccccttttccaagcaaagtctgctcgaggagtctgtaaggtcat |
| | | gtgcttccacccctggtcggatgtaacgctgccactcatgtcggtccctgagatccgg |
| | | gctgttgttgatgcatgggcctcagtcacagaggagctgggtgcccagtacccttggg |
| | | tgcagatctttgaaaacaaaggtgccatgatgggctgttctaacccccacccccactg |
| | | ccaggtatgggccagcagtttcctgccagatattgcccagcgtgaggagcgatctcag |
| | | caggcctataagagtcagcatggagagcccctgctaatggagtacagccgccaggagc |
| | | tactcaggaaggaacgtctggtcctaaccagtgagcactggttagtactggtcccctt |
| | | ctgggcaacatggccctaccagacactgctgctgccccgtcggcatgtgcggcggcta |
| | | cctgagctgacccctgctgagcgtgatgatctagcctccatcatgaagaagctcttga |
| | | ccaagtatgacaacctctttgagacgtcctttccctactccatgggctggcatggggc |
| | | tcccacaggatcagaggctggggccaactggaaccattggcagctgcacgctcattac |
| | | taccctccgctcctgcgctctgccactgtccggaaattcatggttggctacgaaatgc |
| | | ttgctcaggctcagagggacctcacccctgagcaggctgcagagagactaagggcact |
| | | tcctgaggttcattaccacctggggcagaaggacagggagacagcaaccatcgcctga |
| | | cggcgcgccaagcttatcgataatcaacctctggattacaaaatttgtgaaagattga |
| | | ctggtattcttaactatgttgctccttttacgctatgtggatacgctgctttaatgcc |
| | | tttgtatcatgctattgcttcccgtatggctttcattttctcctccttgtataaatcc |
| | | tggtagctgtctctttatgaggagttgtggcccgttgtcaggcaacgtggcgtagtgt |
| | | gcactgtgtttgctgacgcaacccccactggttggggcattgccaccacctgtcagct |
| | | cctttccgggactttcgctttccccctccctattgccacggcggaactcatcgccgcc |
| | | tgccttgcccgctgctggacaggggctcggctgttgggcactgacaattccgtggtgt |
| | | tgtcggggaaatcatcgtcctttccttggctgctcgcctgtgttgccacctggattct |
| | | gcgcgggacgtccttctgctacgtcccttcggccctcaatccagcggaccttccttcc |
| | | cgcggcctgctgccggctctgcggcctcttccgcgtcttcgccttcgccctcagacga |
| | | gtcggatctccctttgggccgcctccccgcatcgataccgagcgctgctcgagagatc |
| | | tacctgtgccttctagttgccagccatctgttgtttgcccctcccccgtgccttcctt |
| | | gaccctggaaggtgccactcccactgtcctttcctaataaaatgaggaaattgcatcg |
| | | cattgtctgagtaggtgtcattctattctggggggtggggtggggcaggacagcaagg |
| | | gggaggattgggaagacaatagcaggcatgctggggatgcggtgggctctatggaggc |
| | | ctaggtgagctctggtaccctctagtcaaggatcaggaacccctagtgatggagttgg |
| | | ccactccctctctgcgcgctcgctcgctcactgaggccgggcgaccaaaggtcgcccg |
| | | acgcccgggctttgcccgggcggcctcagtgagcgagcgagcgcgcagagagggagtg |
| | | gccaa |
| pscAAV- | 6 | tcgcgcgtttcggtgatgacggtgaaaacctctgacacatgcagctcccggagacggt |
| CAG- | | cacagcttgtctgtaagcggatgccgggagcagacaagcccgtcagggcgcgtcagcg |
| hGALT | | ggtgttggcgggtgtcggggctggcttaactatgcggcatcagagcagattgtactga |
| plasmid | | gagtgcaccatatgcggtgtgaaataccgcacagatgcgtaaggagaaaataccgcat |
| | | caggcgccattcgccattcaggctgcgcaactgttgggaagggcgatcggtgcgggcc |
| | | tcttcgctattacgccagctggcgaaagggggatgtgctgcaaggcgattaagttggg |
| | | taacgccagggttttcccagtcacgacgttgtaaaacgacggccagtaggcgcgccca |
| | | aaggctatcaggtcattgcctgagagtctggagcaaacaagagaatcgttggccactc |
| | | cctctctgcgcgctcgctcgctcactgaggccgcccgggcaaagcccgggcgtcgggc |
| | | gacctttggtcgcccggcctcagtgagcgagcgagcgcgcagagagggagtggccaac |
| | | tccatcactaggggttcctgatgtaatacgactcactagttcctggaggggtggagtc |
| | | gtgacctaggccattgacgtcaataatgacgtatgttoccatagtaacgccaataggg |
| | | actttccattgacgtcaatgggggagtatttacggtaaactgcccacttggcagtac |
| | | atcaagtgtatcatatgccaagtacgccccctattgacgtcaatgacggtaaatggcc |
| | | cgcctggcattatgcccagtacatgaccttatgggactttcctacttggcagtacatc |
| | | tacgtattagtcatcgctattaccatggtcgaggtgagccccacgttctgcttcactc |
| | | tccccatctcccccccctccccacccccaattttgtatttatttattttttaattatt |
| | | ttgtgcagcgatgggggcggggggggggggggggcgcgcgccaggcggggcggggcgg |
| | | ggcgaggggcggggcggggcgaggcggagaggtgcggcggcagccaatcagagcggcg |
| | | cgctccgaaagtttccttttatggcgaggcggcggcggcggcggccctataaaaagcg |
| | | aagcgcgcggcgggcgggagtcgctgcgcgctgccttcgccccgtgccccgctccgcc |
| | | gccgcctcgcgccgcccgccccggctctgactgaccgcgttactcccacaggtgagcg |
| | | ggcgggacggcccttctcctccgggctgtaattagcgcttgggaattcgccaccatga |
| | | gcagaagcggcaccgaccctcagcagagacagcaggcctctgaagccgatgccgccgc |
| | | tgccaccttcagagccaatgaccaccagcacatccggtacaaccccctgcaggacgag |
| | | tgggtgctggtgtccgcccacagaatgaagaggccttggcagggccaggtggaacccc |
| | | agctgctgaaaaccgtgcccagacacgaccccctgaaccctctgtgtcctggcgccat |
| | | tagagccaacggcgaagtgaacccccagtacgacagcaccttcctgttcgacaacgac |
| | | ttccccgccctgcagcctgatgccccatctcctggacctagcgaccaccctctgttcc |
| | | aggccaagtctgccagaggcgtgtgcaaagtgatgtgcttccacccttggagcgacgt |
| | | gaccctgcccctgatgagcgtgccagagatcagagccgtggtggatgcctgggccagc |
| | | gtgacagaagaactgggagcccagtacccctgggtgcagatcttcgagaacaagggcg |
| | | ccatgatgggctgcagcaacccccaccctcactgtcaagtgtgggccagcagcttcct |
| | | gcccgatatcgcccagcgggaagagagaagccagcaggcttacaagagccagcacggc |
| | | gagcccctgctgatggaatactccagacaggaactgctgcggaaagaacggctggtgc |
| | | tgaccagcgagcactggctggtgctggtgcctttttgggccacatggccctaccagac |
| | | cctgctgctgcctagaaggcacgtgcggagactgcctgagctgacacccgccgagaga |
| | | gatgacctggccagcatcatgaagaaactgctgaccaaatacgacaacctgttcgaga |
| | | ccagcttcccctacagcatgggctggcacggcgctcctacaggatctgaggctggcgc |
| | | caactggaaccactggcagctgcacgcccactactaccccccactgctgagatctgcc |
| | | accgtgcggaagttcatggtgggatacgagatgctggctcaggcccagagagatctga |
| | | cccctgaacaggccgccgaacggctgagagcactgcccgaagtgcactaccacctggg |
| | | acagaaggacagagagacagccacaatcgcctgaagcgctgctcgagagatctacaac |
| | | ttgtttattgcagcttataatggttacaaataaagcaatagcatcacaaatttcacaa |
| | | ataaagcatttttttcactgcattctagttgtggtttgtccaaactcatcaatgtatc |
| | | ttaaggcctaggtgagctctggtaccctctccactccctctctgcgcgctcgctcgct |
| | | cactgaggccgggcgaccaaaggtcgcccgacgcccgggctttgcccgggcggcctca |
| | | gtgagcgagcgagcgcgcagctgcattaatgaatcggccaacgcgcggggagaggcgg |
| | | tttgcgtattggttaattaacatggtcatagctgtttcctgtgtgaaattgttatccg |
| | | ctcacaattccacacaacatacgagccggaagcataaagtgtaaagcctggggtgcct |
| | | aatgagtgagctaactcacattaattgcgttgcgctcactgcccgctttccagtcggg |
| | | aaacctgtcgtgccagctgcattaatgaatcggccaacgcgcggggagaggcggtttg |
| | | cgtattgggcgctcttccgcttcctcgctcactgactcgctgcgctcggtcgttcggc |
| | | tgcggcgagcggtatcagctcactcaaaggcggtaatacggttatccacagaatcagg |
| | | ggataacgcaggaaagaacatgtgagcaaaaggccagcaaaaggccaggaaccgtaaa |
| | | aaggccgcgttgctggcgtttttccataggctccgcccccctgacgagcatcacaaaa |
| | | atcgacgctcaagtcagaggtggcgaaacccgacaggactataaagataccaggcgtt |
| | | tccccctggaagctccctcgtgcgctctcctgttccgaccctgccgcttaccggatac |
| | | ctgtccgcctttctcccttcgggaagcgtggcgctttctcatagctcacgctgtaggt |
| | | atctcagttcggtgtaggtcgttcgctccaagctgggctgtgtgcacgaaccccccgt |
| | | tcagcccgaccgctgcgccttatccggtaactatcgtcttgagtccaacccggtaaga |
| | | cacgacttatcgccactggcagcagccactggtaacaggattagcagagcgaggtatg |
| | | taggcggtgctacagagttcttgaagtggtggcctaactacggctacactagaagaac |
| | | agtatttggtatctgcgctctgctgaagccagttaccttcggaaaaagagttggtagc |
| | | tcttgatccggcaaacaaaccaccgctggtagcggtggtttttttgtttgcaagcagc |
| | | agattacgcgcagaaaaaaaggatctcaagaagatcctttgatcttttctacggggtc |
| | | tgacgctcagtggaacgaaaactcacgttaagggattttggtcatgagattatcaaaa |
| | | aggatcttcacctagatccttttaaattaaaaatgaagttttaaatcaatctaaagta |
| | | tatatgagtaaacttggtctgacagttagaaaaactcatcgagcatcaaatgaaactg |
| | | caatttattcatatcaggattatcaataccatatttttgaaaaagccgtttctgtaat |
| | | gaaggagaaaactcaccgaggcagttccataggatggcaagatcctggtatcggtctg |
| | | cgattccgactcgtccaacatcaatacaacctattaatttcccctogtcaaaaataag |
| | | gttatcaagtgagaaatcaccatgagtgacgactgaatccggtgagaatggcaaaagt |
| | | ttatgcatttctttccagacttgttcaacaggccagccattacgctcgtcatcaaaat |
| | | cactcgcatcaaccaaaccgttattcattcgtgattgcgcctgagcgagacgaaatac |
| | | gcgatcgctgttaaaaggacaattacaaacaggaatcgaatgcaaccggcgcaggaac |
| | | actgccagcgcatcaacaatattttcacctgaatcaggatattcttctaatacctgga |
| | | atgctgtttttccggggatcgcagtggtgagtaaccatgcatcatcaggagtacggat |
| | | aaaatgcttgatggtcggaagaggcataaattccgtcagccagtttagtctgaccatc |
| | | tcatctgtaacatcattggcaacgctacctttgccatgtttcagaaacaactctggcg |
| | | catcgggcttcccatacaagcgatagattgtcgcacctgattgcccgacattatcgcg |
| | | agcccatttatacccatataaatcagcatccatgttggaatttaatcgcggcctcgac |
| | | gtttcccgttgaatatggctcatactcttcctttttcaatattattgaagcatttatc |
| | | agggttattgtctcatgagcggatacatatttgaatgtatttagaaaaataaacaaat |
| | | aggggttccgcgcacatttccccgaaaagtgccacctgacgtctaagaaaccattatt |
| | | atcatgacattaacctataaaaataggcgtatcacgaggccctttcgtc |
| pAAV2IT | 7 | tcgcgcgtttcggtgatgacggtgaaaacctctgacacatgcagctcccggagacggt |
| R-CAG- | | cacagcttgtctgtaagcggatgccgggagcagacaagcccgtcagggcgcgtcagcg |
| hGALT | | ggtgttggcgggtgtcggggctggcttaactatgcggcatcagagcagattgtactga |
| plasmid | | gagtgcaccatatgcggtgtgaaataccgcacagatgcgtaaggagaaaataccgcat |
| | | caggcgccattcgccattcaggctgcgcaactgttgggaagggcgatcggtgcgggcc |
| | | tcttcgctattacgccagctggcgaaagggggatgtgctgcaaggcgattaagttggg |
| | | taacgccagggttttcccagtcacgacgttgtaaaacgacggccagtaggcgcgccca |
| | | aaggctatcaggtcattgcctgagagtctggagcaaacaagagaatcgttggccactc |
| | | cctctctgcgcgctcgctcgctcactgaggccgcccgggcaaagcccgggcgtcgggc |
| | | gacctttggtcgcccggcctcagtgagcgagcgagcgcgcagagagggagtggccaac |
| | | tccatcactaggggttcctgatgtaatacgactcactagttattaatagtaatcaatt |
| | | acggggtcattagttcatagcccatatatggagttccgcgttacataacttacggtaa |
| | | atggcccgcctggctgaccgcccaacgacccccgcccattgacgtcaataatgacgta |
| | | tgttcccatagtaacgccaatagggactttccattgacgtcaatgggtggactattta |
| | | cggtaaactgcccacttggcagtacatcaagtgtatcatatgccaagtacgcccccta |
| | | ttgacgtcaatgacggtaaatggcccgcctggcattatgcccagtacatgaccttatg |
| | | ggactttcctacttggcagtacatctacgtattagtcatcgctattaccatgggtcga |
| | | ggtgagccccacgttctgcttcactctccccatctcccccccctccccacccccaatt |
| | | ttgtatttatttattttttaattattttgtgcagcgatgggggcgggggggggggggg |
| | | cgcgcgccaggcggggcggggcggggcgaggggcggggcggggcgaggcggagaggtg |
| | | cggcggcagccaatcagagcggcgcgctccgaaagtttccttttatggcgaggcggcg |
| | | gcggcggcggccctataaaaagcgaagcgcgcggcgggcgggagtcgctgcgttgcct |
| | | tcgccccgtgccccgctccgcgccgcctcgcgccgcccgccccggctctgactgaccg |
| | | cgttactcccacaggtgagcgggcgggacggcccttctcctccgggctgtaattagcg |
| | | cttggtttaatgacggctcgtttcttttctgtggctgcgtgaaagccttaaagggctc |
| | | cgggagggccctttgtgcgggggggagcggctcggggggtgcgtgcgtgtgtgtgtgc |
| | | gtggggagcgccgcgtgcggcccgcgctgcccggcggctgtgagcgctgcgggcgcgg |
| | | cgcggggctttgtgcgctccgcgtgtgcgcgaggggagcgcggccgggggcggtgccc |
| | | cgcggtgcgggggggctgcgaggggaacaaaggctgcgtgcggggtgtgtgcgtgggg |
| | | gggtgagcagggggtgtgggcgcggcggtcgggctgtaacccccccctgcacccccct |
| | | ccccgagttgctgagcacggcccggcttcgggtgcggggctccgtgcggggcgtggcg |
| | | cggggctcgccgtgccgggcggggggtggcggcaggtgggggtgccgggcggggcggg |
| | | gccgcctcgggccggggagggctcgggggaggggcgcggcggccccggagcgccggcg |
| | | gctgtcgaggcgcggcgagccgcagccattgccttttatggtaatcgtgcgagagggc |
| | | gcagggacttcctttgtcccaaatctggcggagccgaaatctgggaggcgccgccgca |
| | | ccccctctagcgggcgcgggcgaagcggtgcggcgccggcaggaaggaaatgggcggg |
| | | gagggccttcgtgcgtcgccgcgccgccgtccccttctccatctccagcctcggggct |
| | | gccgcagggggacggctgccttcgggggggacggggcagggcggggttcggcttctgg |
| | | cgtgtgaccggcggctctagagcctctgctaaccatgttcatgccttcttctttttcc |
| | | tacaggccaccatgtcgcgcagtggaaccgatcctcagcaacgccagcaggcgtcaga |
| | | ggcggacgccgcagcagcaaccttccgggcaaacgaccatcagcatatccgctacaac |
| | | ccgctgcaggatgagtgggtgctggtgtcagctcaccgcatgaagcggccctggcagg |
| | | gtcaagtggagccccagcttctgaagacagtgccccgccatgaccctctcaaccctct |
| | | gtgtcctggggccatccgagccaacggagaggtgaatccccagtacgatagcaccttc |
| | | ctgtttgacaacgacttcccagctctgcagcctgatgcccccagtccaggacccagtg |
| | | atcatccccttttccaagcaaagtctgctcgaggagtctgtaaggtcatgtgcttcca |
| | | cccctggtcggatgtaacgctgccactcatgtcggtccctgagatccgggctgttgtt |
| | | gatgcatgggcctcagtcacagaggagctgggtgcccagtacccttgggtgcagatct |
| | | ttgaaaacaaaggtgccatgatgggctgttctaacccccacccccactgccaggtatg |
| | | ggccagcagtttcctgccagatattgcccagcgtgaggagcgatctcagcaggcctat |
| | | aagagtcagcatggagagcccctgctaatggagtacagccgccaggagctactcagga |
| | | aggaacgtctggtcctaaccagtgagcactggttagtactggtccccttctgggcaac |
| | | atggccctaccagacactgctgctgccccgtcggcatgtgcggcggctacctgagctg |
| | | acccctgctgagcgtgatgatctagcctccatcatgaagaagctcttgaccaagtatg |
| | | acaacctctttgagacgtcctttccctactccatgggctggcatggggctcccacagg |
| | | atcagaggctggggccaactggaaccattggcagctgcacgctcattactaccctccg |
| | | ctcctgcgctctgccactgtccggaaattcatggttggctacgaaatgcttgctcagg |
| | | ctcagagggacctcacccctgagcaggctgcagagagactaagggcacttcctgaggt |
| | | tcattaccacctggggcagaaggacagggagacagcaaccatcgcctgacggcgcgcc |
| | | aagcttatcgataatcaacctctggattacaaaatttgtgaaagattgactggtattc |
| | | ttaactatgttgctccttttacgctatgtggatacgctgctttaatgcctttgtatca |
| | | tgctattgcttcccgtatggctttcattttctcctccttgtataaatcctggtagctg |
| | | tctctttatgaggagttgtggcccgttgtcaggcaacgtggcgtagtgtgcactgtgt |
| | | ttgctgacgcaacccccactggttggggcattgccaccacctgtcagctcctttccgg |
| | | gactttcgctttccccctccctattgccacggcggaactcatcgccgcctgccttgcc |
| | | cgctgctggacaggggctcggctgttgggcactgacaattccgtggtgttgtcgggga |
| | | aatcatcgtcctttccttggctgctcgcctgtgttgccacctggattctgcgcgggac |
| | | gtccttctgctacgtcccttcggccctcaatccagcggaccttccttcccgcggcctg |
| | | ctgccggctctgcggcctcttccgcgtcttcgccttcgccctcagacgagtcggatct |
| | | ccctttgggccgcctccccgcatcgataccgagcgctgctcgactgtgccttctagtt |
| | | gccagccatctgttgtttgcccctcccccgtgccttccttgaccctggaaggtgccac |
| | | tcccactgtcctttcctaataaaatgaggaaattgcatcgcattgtctgagtaggtgt |
| | | cattctattctggggggtggggtggggcaggacagcaagggggaggattgggaagaca |
| | | atagcaggcatgctggggatgcggtgggctctatgggagatctacaggcctaggtgag |
| | | ctctggtaccctctagtcaaggatcaggaacccctagtgatggagttggccactccct |
| | | ctctgcgcgctcgctcgctcactgaggccgggcgaccaaaggtcgcccgacgcccggg |
| | | ctttgcccgggcggcctcagtgagcgagcgagcgcgcagagagggagtggccaactgc |
| | | attaatgaatcggccaacgcgcggggagaggcggtttgcgtattggttaattaacatg |
| | | gtcatagctgtttcctgtgtgaaattgttatccgctcacaattccacacaacatacga |
| | | gccggaagcataaagtgtaaagcctggggtgcctaatgagtgagctaactcacattaa |
| | | ttgcgttgcgctcactgcccgctttccagtcgggaaacctgtcgtgccagctgcatta |
| | | atgaatcggccaacgcgcggggagaggcggtttgcgtattgggcgctcttccgcttcc |
| | | tcgctcactgactcgctgcgctcggtcgttcggctgcggcgagcggtatcagctcact |
| | | caaaggcggtaatacggttatccacagaatcaggggataacgcaggaaagaacatgtg |
| | | agcaaaaggccagcaaaaggccaggaaccgtaaaaaggccgcgttgctggcgtttttc |
| | | cataggctccgcccccctgacgagcatcacaaaaatcgacgctcaagtcagaggtggc |
| | | gaaacccgacaggactataaagataccaggcgtttccccctggaagctccctcgtgcg |
| | | ctctcctgttccgaccctgccgcttaccggatacctgtccgcctttctcccttcggga |
| | | agcgtggcgctttctcatagctcacgctgtaggtatctcagttcggtgtaggtcgttc |
| | | gctccaagctgggctgtgtgcacgaaccccccgttcagcccgaccgctgcgccttatc |
| | | cggtaactatcgtcttgagtccaacccggtaagacacgacttatcgccactggcagca |
| | | gccactggtaacaggattagcagagcgaggtatgtaggcggtgctacagagttcttga |
| | | agtggtggcctaactacggctacactagaagaacagtatttggtatctgcgctctgct |
| | | gaagccagttaccttcggaaaaagagttggtagctcttgatccggcaaacaaaccacc |
| | | gctggtagcggtggtttttttgtttgcaagcagcagattacgcgcagaaaaaaaggat |
| | | ctcaagaagatcctttgatcttttctacggggtctgacgctcagtggaacgaaaactc |
| | | acgttaagggattttggtcatgagattatcaaaaaggatcttcacctagatcctttta |
| | | aattaaaaatgaagttttaaatcaatctaaagtatatatgagtaaacttggtctgaca |
| | | gttagaaaaactcatcgagcatcaaatgaaactgcaatttattcatatcaggattatc |
| | | aataccatatttttgaaaaagccgtttctgtaatgaaggagaaaactcaccgaggcag |
| | | ttccataggatggcaagatcctggtatcggtctgcgattccgactcgtccaacatcaa |
| | | tacaacctattaatttcccctcgtcaaaaataaggttatcaagtgagaaatcaccatg |
| | | agtgacgactgaatccggtgagaatggcaaaagtttatgcatttctttccagacttgt |
| | | tcaacaggccagccattacgctcgtcatcaaaatcactcgcatcaaccaaaccgttat |
| | | tcattcgtgattgcgcctgagcgagacgaaatacgcgatcgctgttaaaaggacaatt |
| | | acaaacaggaatcgaatgcaaccggcgcaggaacactgccagcgcatcaacaatattt |
| | | tcacctgaatcaggatattcttctaatacctggaatgctgtttttccggggatcgcag |
| | | tggtgagtaaccatgcatcatcaggagtacggataaaatgcttgatggtcggaagagg |
| | | cataaattccgtcagccagtttagtctgaccatctcatctgtaacatcattggcaacg |
| | | ctacctttgccatgtttcagaaacaactctggcgcatcgggcttcccatacaagcgat |
| | | agattgtcgcacctgattgcccgacattatcgcgagcccatttatacccatataaatc |
| | | agcatccatgttggaatttaatcgcggcctcgacgtttcccgttgaatatggctcata |
| | | ctcttcctttttcaatattattgaagcatttatcagggttattgtctcatgagcggat |
| | | acatatttgaatgtatttagaaaaataaacaaataggggttccgcgcacatttccccg |
| | | aaaagtgccacctgacgtctaagaaaccattattatcatgacattaacctataaaaat |
| | | aggcgtatcacgaggccctttcgtc |
| pAAV2IT | 8 | tcgcgcgtttcggtgatgacggtgaaaacctctgacacatgcagctcccggagacggt |
| R-EF1a- | | cacagcttgtctgtaagcggatgccgggagcagacaagcccgtcagggcgcgtcagcg |
| hGALT | | ggtgttggcgggtgtcggggctggcttaactatgcggcatcagagcagattgtactga |
| plasmid | | gagtgcaccatatgcggtgtgaaataccgcacagatgcgtaaggagaaaataccgcat |
| | | caggcgccattcgccattcaggctgcgcaactgttgggaagggcgatcggtgcgggcc |
| | | tcttcgctattacgccagctggcgaaagggggatgtgctgcaaggcgattaagttggg |
| | | taacgccagggttttcccagtcacgacgttgtaaaacgacggccagtaggcgcgccca |
| | | aaggctatcaggtcattgcctgagagtctggagcaaacaagagaatcgttggccactc |
| | | cctctctgcgcgctcgctcgctcactgaggccgcccgggcaaagcccgggcgtcgggc |
| | | gacctttggtcgcccggcctcagtgagcgagcgagcgcgcagagagggagtggccaac |
| | | tccatcactaggggttcctgatgtaatacgactcactagtgctccggtgcccgtcagt |
| | | gggcagagcgcacatcgcccacagtccccgagaagttggggggaggggtcggcaattg |
| | | aaccggtgcctagagaaggtggcgcggggtaaactgggaaagtgatgtcgtgtactgg |
| | | ctccgcctttttcccgagggtgggggagaaccgtatataagtgcagtagtcgccgtga |
| | | acgttctttttcgcaacgggtttgccgccagaacacaggtaagtgccgtgtgtggttc |
| | | ccgcgggcctggcctctttacgggttatggcccttgcgtgccttgaattacttccacg |
| | | cccctggctgcagtacgtgattcttgatcccgagcttcgggttggaagtgggtgggag |
| | | agttcgaggccttgcgcttaaggagccccttcgcctcgtgcttgagttgaggcctggc |
| | | ctgggcgctggggccgccgcgtgcgaatctggtggcaccttcgcgcctgtctcgctgc |
| | | tttcgataagtctctagccatttaaaatttttgatgacctgctgcgacgctttttttc |
| | | tggcaagatagtcttgtaaatgcgggccaagatctgcacactggtatttcggtttttg |
| | | gggccgcgggcggcgacggggcccgtgcgtcccagcgcacatgttcggcgaggcgggg |
| | | cctgcgagcgcggccaccgagaatcggacgggggtagtctcaagctggccggcctgct |
| | | ctggtgcctggcctcgcgccgccgtgtatcgccccgccctgggcggcaaggctggccc |
| | | ggtcggcaccagttgcgtgagcggaaagatggccgcttcccggccctgctgcagggag |
| | | ctcaaaatggaggacgcggcgctcgggagagcgggcgggtgagtcacccacacaaagg |
| | | aaaagggcctttccgtcctcagccgtcgcttcatgtgactccacggagtaccgggcgc |
| | | cgtccaggcacctcgattagttctcgagcttttggagtacgtcgtctttaggttgggg |
| | | ggaggggttttatgcgatggagtttccccacactgagtgggtggagactgaagttagg |
| | | ccagcttggcacttgatgtaattctccttggaatttgccctttttgagtttggatott |
| | | ggttcattctcaagcctcagacagtggttcaaagtttttttcttccatttcaggtgtc |
| | | gtgagccaccatgtcgcgcagtggaaccgatcctcagcaacgccagcaggcgtcagag |
| | | gcggacgccgcagcagcaaccttccgggcaaacgaccatcagcatatccgctacaacc |
| | | cgctgcaggatgagtgggtgctggtgtcagctcaccgcatgaagcggccctggcaggg |
| | | tcaagtggagccccagcttctgaagacagtgccccgccatgaccctctcaaccctctg |
| | | tgtcctggggccatccgagccaacggagaggtgaatccccagtacgatagcaccttcc |
| | | tgtttgacaacgacttcccagctctgcagcctgatgcccccagtccaggacccagtga |
| | | tcatccccttttccaagcaaagtctgctcgaggagtctgtaaggtcatgtgcttccac |
| | | ccctggtcggatgtaacgctgccactcatgtcggtccctgagatccgggctgttgttg |
| | | atgcatgggcctcagtcacagaggagctgggtgcccagtacccttgggtgcagatctt |
| | | tgaaaacaaaggtgccatgatgggctgttctaacccccacccccactgccaggtatgg |
| | | gccagcagtttcctgccagatattgcccagcgtgaggagcgatctcagcaggcctata |
| | | agagtcagcatggagagcccctgctaatggagtacagccgccaggagctactcaggaa |
| | | ggaacgtctggtcctaaccagtgagcactggttagtactggtccccttctgggcaaca |
| | | tggccctaccagacactgctgctgccccgtcggcatgtgcggcggctacctgagctga |
| | | cccctgctgagcgtgatgatctagcctccatcatgaagaagctcttgaccaagtatga |
| | | caacctctttgagacgtcctttccctactccatgggctggcatggggctcccacagga |
| | | tcagaggctggggccaactggaaccattggcagctgcacgctcattactaccctccgc |
| | | tcctgcgctctgccactgtccggaaattcatggttggctacgaaatgcttgctcaggc |
| | | tcagagggacctcacccctgagcaggctgcagagagactaagggcacttcctgaggtt |
| | | cattaccacctggggcagaaggacagggagacagcaaccatcgcctgacggcgcgcca |
| | | agcttatcgataatcaacctctggattacaaaatttgtgaaagattgactggtattct |
| | | taactatgttgctccttttacgctatgtggatacgctgctttaatgcctttgtatcat |
| | | gctattgcttcccgtatggctttcattttctcctccttgtataaatcctggtagctgt |
| | | ctctttatgaggagttgtggcccgttgtcaggcaacgtggcgtagtgtgcactgtgtt |
| | | tgctgacgcaacccccactggttggggcattgccaccacctgtcagctcctttccggg |
| | | actttcgctttccccctccctattgccacggcggaactcatcgccgcctgccttgccc |
| | | gctgctggacaggggctcggctgttgggcactgacaattccgtggtgttgtcggggaa |
| | | atcatcgtcctttccttggctgctcgcctgtgttgccacctggattctgcgcgggacg |
| | | tccttctgctacgtcccttcggccctcaatccagcggaccttccttcccgcggcctgc |
| | | tgccggctctgcggcctcttccgcgtcttcgccttcgccctcagacgagtcggatctc |
| | | cctttgggccgcctccccgcatcgataccgagcgctgctcgagagatctacctgtgcc |
| | | ttctagttgccagccatctgttgtttgcccctcccccgtgccttccttgaccctggaa |
| | | ggtgccactcccactgtcctttcctaataaaatgaggaaattgcatcgcattgtctga |
| | | gtaggtgtcattctattctggggggtggggtggggcaggacagcaagggggaggattg |
| | | ggaagacaatagcaggcatgctggggatgcggtgggctctatggaggcctaggtgagc |
| | | tctggtaccctctagtcaaggatcaggaacccctagtgatggagttggccactccctc |
| | | tctgcgcgctcgctcgctcactgaggccgggcgaccaaaggtcgcccgacgcccgggc |
| | | tttgcccgggcggcctcagtgagcgagcgagcgcgcagagagggagtggccaactgca |
| | | ttaatgaatcggccaacgcgcggggagaggcggtttgcgtattggttaattaacatgg |
| | | tcatagctgtttcctgtgtgaaattgttatccgctcacaattccacacaacatacgag |
| | | ccggaagcataaagtgtaaagcctggggtgcctaatgagtgagctaactcacattaat |
| | | tgcgttgcgctcactgcccgctttccagtcgggaaacctgtcgtgccagctgcattaa |
| | | tgaatcggccaacgcgcggggagaggcggtttgcgtattgggcgctcttccgcttcct |
| | | cgctcactgactcgctgcgctcggtcgttcggctgcggcgagcggtatcagctcactc |
| | | aaaggcggtaatacggttatccacagaatcaggggataacgcaggaaagaacatgtga |
| | | gcaaaaggccagcaaaaggccaggaaccgtaaaaaggccgcgttgctggcgtttttcc |
| | | ataggctccgcccccctgacgagcatcacaaaaatcgacgctcaagtcagaggtggcg |
| | | aaacccgacaggactataaagataccaggcgtttccccctggaagctccctcgtgcgc |
| | | tctcctgttccgaccctgccgcttaccggatacctgtccgcctttctcccttcgggaa |
| | | gcgtggcgctttctcatagctcacgctgtaggtatctcagttcggtgtaggtcgttcg |
| | | ctccaagctgggctgtgtgcacgaaccccccgttcagcccgaccgctgcgccttatcc |
| | | ggtaactatcgtcttgagtccaacccggtaagacacgacttatcgccactggcagcag |
| | | ccactggtaacaggattagcagagcgaggtatgtaggcggtgctacagagttcttgaa |
| | | gtggtggcctaactacggctacactagaagaacagtatttggtatctgcgctctgctg |
| | | aagccagttaccttcggaaaaagagttggtagctcttgatccggcaaacaaaccaccg |
| | | ctggtagcggtggtttttttgtttgcaagcagcagattacgcgcagaaaaaaaggatc |
| | | tcaagaagatcctttgatcttttctacggggtctgacgctcagtggaacgaaaactca |
| | | cgttaagggattttggtcatgagattatcaaaaaggatcttcacctagatccttttaa |
| | | attaaaaatgaagttttaaatcaatctaaagtatatatgagtaaacttggtctgacag |
| | | ttagaaaaactcatcgagcatcaaatgaaactgcaatttattcatatcaggattatca |
| | | ataccatatttttgaaaaagccgtttctgtaatgaaggagaaaactcaccgaggcagt |
| | | tccataggatggcaagatcctggtatcggtctgcgattccgactcgtccaacatcaat |
| | | acaacctattaatttcccctcgtcaaaaataaggttatcaagtgagaaatcaccatga |
| | | gtgacgactgaatccggtgagaatggcaaaagtttatgcatttctttccagacttgtt |
| | | caacaggccagccattacgctcgtcatcaaaatcactcgcatcaaccaaaccgttatt |
| | | cattcgtgattgcgcctgagcgagacgaaatacgcgatcgctgttaaaaggacaatta |
| | | caaacaggaatcgaatgcaaccggcgcaggaacactgccagcgcatcaacaatatttt |
| | | cacctgaatcaggatattcttctaatacctggaatgctgtttttccggggatcgcagt |
| | | ggtgagtaaccatgcatcatcaggagtacggataaaatgcttgatggtcggaagaggc |
| | | ataaattccgtcagccagtttagtctgaccatctcatctgtaacatcattggcaacgc |
| | | tacctttgccatgtttcagaaacaactctggcgcatcgggcttcccatacaagcgata |
| | | gattgtcgcacctgattgcccgacattatcgcgagcccatttatacccatataaatca |
| | | gcatccatgttggaatttaatcgcggcctcgacgtttcccgttgaatatggctcatac |
| | | tcttcctttttcaatattattgaagcatttatcagggttattgtctcatgagcggata |
| | | catatttgaatgtatttagaaaaataaacaaataggggttccgcgcacatttccccga |
| | | aaagtgccacctgacgtctaagaaaccattattatcatgacattaacctataaaaata |
| | | ggcgtatcacgaggccctttcgtc |
| CAG | 9 | ccattgacgtcaataatgacgtatgttcccatagtaacgccaatagggactttccatt |
| promoter | | gacgtcaatgggtggagtatttacggtaaactgcccacttggcagtacatcaagtgta |
| | | tcatatgccaagtacgccccctattgacgtcaatgacggtaaatggcccgcctggcat |
| | | tatgcccagtacatgaccttatgggactttcctacttggcagtacatctacgtattag |
| | | tcatcgctattaccatggtcgaggtgagccccacgttctgcttcactctccccatctc |
| | | ccccccctccccacccccaattttgtatttatttattttttaattattttgtgcagcg |
| | | atgggggcggggggggggggggggcgcgcgccaggcggggcggggcggggcgaggggc |
| | | ggggcggggcgaggcggagaggtgcggcggcagccaatcagagcggcgcgctccgaaa |
| | | gtttccttttatggcgaggcggcggcggcggcggccctataaaaagcgaagcgcgcgg |
| | | cgggcgggagtcgctgcgcgctgccttcgccccgtgccccgctccgccgccgcctcgc |
| | | gccgcccgccccggctctgactgaccgcgttactcccacaggtgagcgggcgggacgg |
| | | cccttctcctccgggctgtaattagcgcttgg |
| EF1alpha | 10 | gctccggtgcccgtcagtgggcagagcgcacatcgcccacagtccccgagaagttggg |
| promoter | | gggaggggtcggcaattgaaccggtgcctagagaaggtggcgcggggtaaactgggaa |
| (including | | agtgatgtcgtgtactggctccgcctttttcccgagggtgggggagaaccgtatataa |
| Intron A) | | gtgcagtagtcgccgtgaacgttctttttcgcaacgggtttgccgccagaacacaggt |
| | | aagtgccgtgtgtggttcccgcgggcctggcctctttacgggttatggcccttgcgtg |
| | | ccttgaattacttccacgcccctggctgcagtacgtgattcttgatcccgagcttcgg |
| | | gttggaagtgggtgggagagttcgaggccttgcgcttaaggagccccttcgcctcgtg |
| | | cttgagttgaggcctggcctgggcgctggggccgccgcgtgcgaatctggtggcacct |
| | | tcgcgcctgtctcgctgctttcgataagtctctagccatttaaaatttttgatgacct |
| | | gctgcgacgctttttttctggcaagatagtcttgtaaatgcgggccaagatctgcaca |
| | | ctggtatttcggtttttggggccgcgggcggcgacggggcccgtgcgtcccagcgcac |
| | | atgttcggcgaggcggggcctgcgagcgcggccaccgagaatcggacgggggtagtct |
| | | caagctggccggcctgctctggtgcctggcctcgcgccgccgtgtatcgccccgccct |
| | | gggcggcaaggctggcccggtcggcaccagttgcgtgagcggaaagatggccgcttcc |
| | | cggccctgctgcagggagctcaaaatggaggacgcggcgctcgggagagcgggcgggt |
| | | gagtcacccacacaaaggaaaagggcctttccgtcctcagccgtcgcttcatgtgact |
| | | ccacggagtaccgggcgccgtccaggcacctcgattagttctcgagcttttggagtac |
| | | gtcgtctttaggttggggggaggggttttatgcgatggagtttccccacactgagtgg |
| | | gtggagactgaagttaggccagcttggcacttgatgtaattctccttggaatttgccc |
| | | tttttgagtttggatcttggttcattctcaagcctcagacagtggttcaaagtttttt |
| | | tcttccatttcaggtgtcgtga |
| 5′ AAV2 | 11 | ttggccactccctctctgcgcgctcgctcgctcactgaggccgcccgggcaaagcccg |
| ITR | | ggcgtcgggcgacctttggtcgcccggcctcagtgagcgagcgagcgcgcagagaggg |
| | | agtggccaactccatcactaggggttcct |
| 3′ AAV2 | 12 | aggaacccctagtgatggagttggccactccctctctgcgcgctcgctcgctcactga |
| ITR | | ggccgggcgaccaaaggtcgcccgacgcccgggctttgcccgggcggcctcagtgagc |
| | | gagcgagcgcgcagagagggagtggccaa |
| Self- | 13 | ccactccctctctgcgcgctcgctcgctcactgaggccgggcgaccaaaggtcgcccg |
| complement- | | acgcccgggctttgcccgggcggcctcagtgagcgagcgagcgcgcag |
| ary ITR | | |
| WPRE | 14 | aatcaacctctggattacaaaatttgtgaaagattgactggtattcttaactatgttg |
| element | | ctccttttacgctatgtggatacgctgctttaatgcctttgtatcatgctattgcttc |
| | | ccgtatggctttcattttctcctccttgtataaatcctggtagctgtctctttatgag |
| | | gagttgtggcccgttgtcaggcaacgtggcgtagtgtgcactgtgtttgctgacgcaa |
| | | cccccactggttggggcattgccaccacctgtcagctcctttccgggactttcgcttt |
| | | ccccctccctattgccacggcggaactcatcgccgcctgccttgcccgctgctggaca |
| | | ggggctcggctgttgggcactgacaattccgtggtgttgtcggggaaatcatcgtcct |
| | | ttccttggctgctcgcctgtgttgccacctggattctgcgcgggacgtccttctgcta |
| | | cgtcccttcggccctcaatccagcggaccttccttcccgcggcctgctgccggctctg |
| | | cggcctcttccgcgtcttcgccttcgccctcagacgagtcggatctccctttgggccg |
| | | cctccccgc |
| bGH poly | 15 | ctgtgccttctagttgccagccatctgttgtttgcccctcccccgtgccttccttgac |
| A signal | | cctggaaggtgccactcccactgtcctttcctaataaaatgaggaaattgcatcgcat |
| | | tgtctgagtaggtgtcattctattctggggggtggggtggggcaggacagcaaggggg |
| | | aggattgggaagacaatagcaggcatgctggggatgcggtgggctctatgg |
| SV40 poly | 16 | aacttgtttattgcagcttataatggttacaaataaagcaatagcatcacaaatttca |
| A signal | | caaataaagcatttttttcactgcattctagttgtggtttgtccaaactcatcaatgt |
| | | atctta |
| Chimeric | 17 | ggagtcgctgcgttgccttcgccccgtgccccgctccgcgccgcctcgcgccgcccgc |
| intron | | cccggctctgactgaccgcgttactcccacaggtgagcgggcgggacggcccttctcc |
| | | tccgggctgtaattagcgcttggtttaatgacggctcgtttcttttctgtggctgcgt |
| | | gaaagccttaaagggctccgggagggccctttgtgcgggggggagcggctcggggggt |
| | | gcgtgcgtgtgtgtgtgcgtggggagcgccgcgtgcggcccgcgctgcccggcggctg |
| | | tgagcgctgcgggcgcggcgcggggctttgtgcgctccgcgtgtgcgcgaggggagcg |
| | | cggccgggggcggtgccccgcggtgcgggggggctgcgaggggaacaaaggctgcgtg |
| | | cggggtgtgtgcgtgggggggtgagcagggggtgtgggcgcggcggtcgggctgtaac |
| | | ccccccctgcacccccctccccgagttgctgagcacggcccggcttcgggtgcggggc |
| | | tccgtgcggggcgtggcgcggggctcgccgtgccgggcgggcgggggcggcaggtggg |
| | | ggtgccgggcggggcggggccgcctcgggccggggagggctcgggggaggggcgcggc |
| | | ggccccggagcgccggcggctgtcgaggcgcggcgagccgcagccattgccttttatg |
| | | gtaatcgtgcgagagggcgcagggacttcctttgtcccaaatctggcggagccgaaat |
| | | ctgggaggcgccgccgcaccccctctagcgggcgcgggcgaagcggtgcggcgccggc |
| | | aggaaggaaatgggcggggagggccttcgtgcgtcgccgcgccgccgtccccttctcc |
| | | atctccagcctcggggctgccgcagggggacggctgccttcgggggggacggggcagg |
| | | gcggggttcggcttctggcgtgtgaccggcggctctagagcctctgctaaccatgttc |
| | | atgccttcttctttttcctacag |
| AAV9 | 18 | MAADGYLPDW LEDNLSEGIR EWWALKPGAP QPKANQQHQD NARGLVLPGY |
| capsid | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDENRFH CHFSPRDWQR |
| | | LINNNWGFRP KRLNFKLFNI QVKEVIDNNG VKTIANNLTS TVQVFTDSDY |
| | | QLPYVLGSAH EGCLPPFPAD VFMIPQYGYL TLNDGSQAVG RSSFYCLEYF |
| | | PSQMLRTGNN FQFSYEFENV PFHSSYAHSQ SLDRLMNPLI DQYLYYLSKT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSAQAQ AQTGWVQNQG |
| | | ILPGMVWQDR DVYLQGPIWA KIPHTDGNFH PSPLMGGFGM KHPPPQILIK |
| | | NTPVPADPPT AFNKDKLNSF ITQYSTGQVS VEIEWELQKE NSKRWNPEIQ |
| | | YTSNYYKSNN VEFAVNTEGV YSEPRPIGTR YLTRNL |
| pscAAV- | 19 | ccattgacgtcaataatgacgtatgttoccatagtaacgccaatagggactttccatt |
| CAG- | | gacgtcaatgggtggagtatttacggtaaactgcccacttggcagtacatcaagtgta |
| hGALT | | tcatatgccaagtacgccccctattgacgtcaatgacggtaaatggcccgcctggcat |
| expression | | tatgcccagtacatgaccttatgggactttcctacttggcagtacatctacgtattag |
| cassette | | tcatcgctattaccatggtcgaggtgagccccacgttctgcttcactctccccatctc |
| (not | | ccccccctccccacccccaattttgtatttatttattttttaattattttgtgcagcg |
| including | | atgggggcggggggggggggggggcgcgcgccaggcggggcggggcggggcgaggggc |
| flanking | | ggggcggggcgaggcggagaggtgcggcggcagccaatcagagcggcgcgctccgaaa |
| ITRs) | | gtttccttttatggcgaggcggcggcggcggcggccctataaaaagcgaagcgcgcgg |
| | | cgggcgggagtcgctgcgcgctgccttcgccccgtgccccgctccgccgccgcctcgc |
| | | gccgcccgccccggctctgactgaccgcgttactcccacaggtgagcgggcgggacgg |
| | | cccttctcctccgggctgtaattagcgcttgggaattcgccaccatgagcagaagcgg |
| | | caccgaccctcagcagagacagcaggcctctgaagccgatgccgccgctgccaccttc |
| | | agagccaatgaccaccagcacatccggtacaaccccctgcaggacgagtgggtgctgg |
| | | tgtccgcccacagaatgaagaggccttggcagggccaggtggaaccccagctgctgaa |
| | | aaccgtgcccagacacgaccccctgaaccctctgtgtcctggcgccattagagccaac |
| | | ggcgaagtgaacccccagtacgacagcaccttcctgttcgacaacgacttccccgccc |
| | | tgcagcctgatgccccatctcctggacctagcgaccaccctctgttccaggccaagtc |
| | | tgccagaggcgtgtgcaaagtgatgtgcttccacccttggagcgacgtgaccctgccc |
| | | ctgatgagcgtgccagagatcagagccgtggtggatgcctgggccagcgtgacagaag |
| | | aactgggagcccagtacccctgggtgcagatcttcgagaacaagggcgccatgatggg |
| | | ctgcagcaacccccaccctcactgtcaagtgtgggccagcagcttcctgcccgatatc |
| | | gcccagcgggaagagagaagccagcaggcttacaagagccagcacggcgagcccctgc |
| | | tgatggaatactccagacaggaactgctgcggaaagaacggctggtgctgaccagcga |
| | | gcactggctggtgctggtgcctttttgggccacatggccctaccagaccctgctgctg |
| | | cctagaaggcacgtgcggagactgcctgagctgacacccgccgagagagatgacctgg |
| | | ccagcatcatgaagaaactgctgaccaaatacgacaacctgttcgagaccagcttccc |
| | | ctacagcatgggctggcacggcgctcctacaggatctgaggctggcgccaactggaac |
| | | cactggcagctgcacgcccactactaccccccactgctgagatctgccaccgtgcgga |
| | | agttcatggtgggatacgagatgctggctcaggcccagagagatctgacccctgaaca |
| | | ggccgccgaacggctgagagcactgcccgaagtgcactaccacctgggacagaaggac |
| | | agagagacagccacaatcgcctgaAGCGCTGCTCGAGAGATCTACAACTTGTTTATTG |
| | | CAGCTTATAATGGTTACAAATAAAGCAATAGCATCACAAATTTCACAAATAAAGCATT |
| | | TTTTTCACTGCATTCTAGTTGTGGTTTGTCCAAACTCATCAATGTATCTTA |
| pAAV2IT | 20 | CGTTACATAACTTACGGTAAATGGCCCGCCTGGCTGACCGCCCAACGACCCCCGCCCA |
| R-CAG- | | TTGACGTCAATAATGACGTATGTTCCCATAGTAACGCCAATAGGGACTTTCCATTGAC |
| hGALT | | GTCAATGGGTGGACTATTTACGGTAAACTGCCCACTTGGCAGTACATCAAGTGTATCA |
| expression | | TATGCCAAGTACGCCCCCTATTGACGTCAATGACGGTAAATGGCCCGCCTGGCATTAT |
| cassette | | GCCCAGTACATGACCTTATGGGACTTTCCTACTTGGCAGTACATCTACGTATTAGTCA |
| (not | | TCGCTATTACCATGGGTCGAGGTGAGCCCCACGTTCTGCTTCACTCTCCCCATCTCCC |
| including | | CCCCCTCCCCACCCCCAATTTTGTATTTATTTATTTTTTAATTATTTTGTGCAGCGAT |
| flanking | | GGGGGCGGGGGGGGGGGGGGCGCGCGCCAGGCGGGGCGGGGCGGGGCGAGGGGCGGGG |
| ITRs) | | CGGGGCGAGGCGGAGAGGTGCGGCGGCAGCCAATCAGAGCGGCGCGCTCCGAAAGTTT |
| | | CCTTTTATGGCGAGGCGGCGGCGGCGGCGGCCCTATAAAAAGCGAAGCGCGCGGCGGG |
| | | CGGGAGTCGCTGCGTTGCCTTCGCCCCGTGCCCCGCTCCGCGCCGCCTCGCGCCGCCC |
| | | GCCCCGGCTCTGACTGACCGCGTTACTCCCACAGGTGAGCGGGCGGGACGGCCCTTCT |
| | | CCTCCGGGCTGTAATTAGCGCTTGGTTTAATGACGGCTCGTTTCTTTTCTGTGGCTGC |
| | | GTGAAAGCCTTAAAGGGCTCCGGGAGGGCCCTTTGTGCGGGGGGGAGCGGCTCGGGGG |
| | | GTGCGTGCGTGTGTGTGTGCGTGGGGAGCGCCGCGTGCGGCCCGCGCTGCCCGGCGGC |
| | | TGTGAGCGCTGCGGGCGCGGCGCGGGGCTTTGTGCGCTCCGCGTGTGCGCGAGGGGAG |
| | | CGCGGCCGGGGGCGGTGCCCCGCGGTGCGGGGGGGCTGCGAGGGGAACAAAGGCTGCG |
| | | TGCGGGGTGTGTGCGTGGGGGGGTGAGCAGGGGGTGTGGGCGCGGCGGTCGGGCTGTA |
| | | ACCCCCCCCTGCACCCCCCTCCCCGAGTTGCTGAGCACGGCCCGGCTTCGGGTGCGGG |
| | | GCTCCGTGCGGGGCGTGGCGCGGGGCTCGCCGTGCCGGGGGGGGGGTGGCGGCAGGTG |
| | | GGGGTGCCGGGGGGGGGGGGGCCGCCTCGGGCCGGGGAGGGCTCGGGGGAGGGGCGCG |
| | | GCGGCCCCGGAGCGCCGGCGGCTGTCGAGGCGCGGCGAGCCGCAGCCATTGCCTTTTA |
| | | TGGTAATCGTGCGAGAGGGCGCAGGGACTTCCTTTGTCCCAAATCTGGCGGAGCCGAA |
| | | ATCTGGGAGGCGCCGCCGCACCCCCTCTAGCGGGCGCGGGCGAAGCGGTGCGGCGCCG |
| | | GCAGGAAGGAAATGGGGGGGGAGGGCCTTCGTGCGTCGCCGCGCCGCCGTCCCCTTCT |
| | | CCATCTCCAGCCTCGGGGCTGCCGCAGGGGGACGGCTGCCTTCGGGGGGGACGGGGCA |
| | | GGGCGGGGTTCGGCTTCTGGCGTGTGACCGGCGGCTCTAGAGCCTCTGCTAACCATGT |
| | | TCATGCCTTCTTCTTTTTCCTACAGGCCACCATGTCGCGCAGTGGAACCGATCCTCAG |
| | | CAACGCCAGCAGGCGTCAGAGGCGGACGCCGCAGCAGCAACCTTCCGGGCAAACGACC |
| | | ATCAGCATATCCGCTACAACCCGCTGCAGGATGAGTGGGTGCTGGTGTCAGCTCACCG |
| | | CATGAAGCGGCCCTGGCAGGGTCAAGTGGAGCCCCAGCTTCTGAAGACAGTGCCCCGC |
| | | CATGACCCTCTCAACCCTCTGTGTCCTGGGGCCATCCGAGCCAACGGAGAGGTGAATC |
| | | CCCAGTACGATAGCACCTTCCTGTTTGACAACGACTTCCCAGCTCTGCAGCCTGATGC |
| | | CCCCAGTCCAGGACCCAGTGATCATCCCCTTTTCCAAGCAAAGTCTGCTCGAGGAGTC |
| | | TGTAAGGTCATGTGCTTCCACCCCTGGTCGGATGTAACGCTGCCACTCATGTCGGTCC |
| | | CTGAGATCCGGGCTGTTGTTGATGCATGGGCCTCAGTCACAGAGGAGCTGGGTGCCCA |
| | | GTACCCTTGGGTGCAGATCTTTGAAAACAAAGGTGCCATGATGGGCTGTTCTAACCCC |
| | | CACCCCCACTGCCAGGTATGGGCCAGCAGTTTCCTGCCAGATATTGCCCAGCGTGAGG |
| | | AGCGATCTCAGCAGGCCTATAAGAGTCAGCATGGAGAGCCCCTGCTAATGGAGTACAG |
| | | CCGCCAGGAGCTACTCAGGAAGGAACGTCTGGTCCTAACCAGTGAGCACTGGTTAGTA |
| | | CTGGTCCCCTTCTGGGCAACATGGCCCTACCAGACACTGCTGCTGCCCCGTCGGCATG |
| | | TGCGGCGGCTACCTGAGCTGACCCCTGCTGAGCGTGATGATCTAGCCTCCATCATGAA |
| | | GAAGCTCTTGACCAAGTATGACAACCTCTTTGAGACGTCCTTTCCCTACTCCATGGGC |
| | | TGGCATGGGGCTCCCACAGGATCAGAGGCTGGGGCCAACTGGAACCATTGGCAGCTGC |
| | | ACGCTCATTACTACCCTCCGCTCCTGCGCTCTGCCACTGTCCGGAAATTCATGGTTGG |
| | | CTACGAAATGCTTGCTCAGGCTCAGAGGGACCTCACCCCTGAGCAGGCTGCAGAGAGA |
| | | CTAAGGGCACTTCCTGAGGTTCATTACCACCTGGGGCAGAAGGACAGGGAGACAGCAA |
| | | CCATCGCCTGACGGCGCGCCAAGCTTATCGATAATCAACCTCTGGATTACAAAATTTG |
| | | TGAAAGATTGACTGGTATTCTTAACTATGTTGCTCCTTTTACGCTATGTGGATACGCT |
| | | GCTTTAATGCCTTTGTATCATGCTATTGCTTCCCGTATGGCTTTCATTTTCTCCTCCT |
| | | TGTATAAATCCTGGTAGCTGTCTCTTTATGAGGAGTTGTGGCCCGTTGTCAGGCAACG |
| | | TGGCGTAGTGTGCACTGTGTTTGCTGACGCAACCCCCACTGGTTGGGGCATTGCCACC |
| | | ACCTGTCAGCTCCTTTCCGGGACTTTCGCTTTCCCCCTCCCTATTGCCACGGCGGAAC |
| | | TCATCGCCGCCTGCCTTGCCCGCTGCTGGACAGGGGCTCGGCTGTTGGGCACTGACAA |
| | | TTCCGTGGTGTTGTCGGGGAAATCATCGTCCTTTCCTTGGCTGCTCGCCTGTGTTGCC |
| | | ACCTGGATTCTGCGCGGGACGTCCTTCTGCTACGTCCCTTCGGCCCTCAATCCAGCGG |
| | | ACCTTCCTTCCCGCGGCCTGCTGCCGGCTCTGCGGCCTCTTCCGCGTCTTCGCCTTCG |
| | | CCCTCAGACGAGTCGGATCTCCCTTTGGGCCGCCTCCCCGCATCGATACCGAGCGCTG |
| | | CTCGACTGTGCCTTCTAGTTGCCAGCCATCTGTTGTTTGCCCCTCCCCCGTGCCTTCC |
| | | TTGACCCTGGAAGGTGCCACTCCCACTGTCCTTTCCTAATAAAATGAGGAAATTGCAT |
| | | CGCATTGTCTGAGTAGGTGTCATTCTATTCTGGGGGGTGGGGTGGGGCAGGACAGCAA |
| | | GGGGGAGGATTGGGAAGACAATAGCAGGCATGCTGGGGATGCGGTGGGCTCTATGG |
| pAAV2IT | 21 | gctccggtgcccgtcagtgggcagagcgcacatcgcccacagtccccgagaagttggg |
| R-EF1a- | | gggaggggtcggcaattgaaccggtgcctagagaaggtggcgcggggtaaactgggaa |
| hGALT | | agtgatgtcgtgtactggctccgcctttttcccgagggtgggggagaaccgtatataa |
| expression | | gtgcagtagtcgccgtgaacgttctttttcgcaacgggtttgccgccagaacacaggt |
| cassette | | aagtgccgtgtgtggttcccgcgggcctggcctctttacgggttatggcccttgcgtg |
| (not | | ccttgaattacttccacgcccctggctgcagtacgtgattcttgatcccgagcttcgg |
| including | | gttggaagtgggtgggagagttcgaggccttgcgcttaaggagccccttcgcctcgtg |
| flanking | | cttgagttgaggcctggcctgggcgctggggccgccgcgtgcgaatctggtggcacct |
| ITRs) | | tcgcgcctgtctcgctgctttcgataagtctctagccatttaaaatttttgatgacct |
| | | gctgcgacgctttttttctggcaagatagtcttgtaaatgcgggccaagatctgcaca |
| | | ctggtatttcggtttttggggccgcgggcggcgacggggcccgtgcgtcccagcgcac |
| | | atgttcggcgaggcggggcctgcgagcgcggccaccgagaatcggacgggggtagtct |
| | | caagctggccggcctgctctggtgcctggcctcgcgccgccgtgtatcgccccgccct |
| | | gggcggcaaggctggcccggtcggcaccagttgcgtgagcggaaagatggccgcttcc |
| | | cggccctgctgcagggagctcaaaatggaggacgcggcgctcgggagagcgggcgggt |
| | | gagtcacccacacaaaggaaaagggcctttccgtcctcagccgtcgcttcatgtgact |
| | | ccacggagtaccgggcgccgtccaggcacctcgattagttctcgagcttttggagtac |
| | | gtcgtctttaggttggggggaggggttttatgcgatggagtttccccacactgagtgg |
| | | gtggagactgaagttaggccagcttggcacttgatgtaattctccttggaatttgccc |
| | | tttttgagtttggatcttggttcattctcaagcctcagacagtggttcaaagtttttt |
| | | tcttccatttcaggtgtcgtgaGccaccATGTCGCGCAGTGGAACCGATCCTCAGCAA |
| | | CGCCAGCAGGCGTCAGAGGCGGACGCCGCAGCAGCAACCTTCCGGGCAAACGACCATC |
| | | AGCATATCCGCTACAACCCGCTGCAGGATGAGTGGGTGCTGGTGTCAGCTCACCGCAT |
| | | GAAGCGGCCCTGGCAGGGTCAAGTGGAGCCCCAGCTTCTGAAGACAGTGCCCCGCCAT |
| | | GACCCTCTCAACCCTCTGTGTCCTGGGGCCATCCGAGCCAACGGAGAGGTGAATCCCC |
| | | AGTACGATAGCACCTTCCTGTTTGACAACGACTTCCCAGCTCTGCAGCCTGATGCCCC |
| | | CAGTCCAGGACCCAGTGATCATCCCCTTTTCCAAGCAAAGTCTGCTCGAGGAGICTGT |
| | | AAGGTCATGTGCTTCCACCCCTGGTCGGATGTAACGCTGCCACTCATGTCGGTCCCTG |
| | | AGATCCGGGCTGTTGTTGATGCATGGGCCTCAGTCACAGAGGAGCTGGGTGCCCAGTA |
| | | CCCTTGGGTGCAGATCTTTGAAAACAAAGGTGCCATGATGGGCTGTTCTAACCCCCAC |
| | | CCCCACTGCCAGGTATGGGCCAGCAGTTTCCTGCCAGATATTGCCCAGCGTGAGGAGC |
| | | GATCTCAGCAGGCCTATAAGAGTCAGCATGGAGAGCCCCTGCTAATGGAGTACAGCCG |
| | | CCAGGAGCTACTCAGGAAGGAACGTCTGGTCCTAACCAGTGAGCACTGGTTAGTACTG |
| | | GTCCCCTTCTGGGCAACATGGCCCTACCAGACACTGCTGCTGCCCCGTCGGCATGTGC |
| | | GGCGGCTACCTGAGCTGACCCCTGCTGAGCGTGATGATCTAGCCTCCATCATGAAGAA |
| | | GCTCTTGACCAAGTATGACAACCTCTTTGAGACGTCCTTTCCCTACTCCATGGGCTGG |
| | | CATGGGGCTCCCACAGGATCAGAGGCTGGGGCCAACTGGAACCATTGGCAGCTGCACG |
| | | CTCATTACTACCCTCCGCTCCTGCGCTCTGCCACTGTCCGGAAATTCATGGTTGGCTA |
| | | CGAAATGCTTGCTCAGGCTCAGAGGGACCTCACCCCTGAGCAGGCTGCAGAGAGACTA |
| | | AGGGCACTTCCTGAGGTTCATTACCACCTGGGGCAGAAGGACAGGGAGACAGCAACCA |
| | | TCGCCTGACGGCGCGCCAAGCTTATCGATAATCAACCTCTGGATTACAAAATTTGTGA |
| | | AAGATTGACTGGTATTCTTAACTATGTTGCTCCTTTTACGCTATGTGGATACGCTGCT |
| | | TTAATGCCTTTGTATCATGCTATTGCTTCCCGTATGGCTTTCATTTTCTCCTCCTTGT |
| | | ATAAATCCTGGTAGCTGTCTCTTTATGAGGAGTTGTGGCCCGTTGTCAGGCAACGTGG |
| | | CGTAGTGTGCACTGTGTTTGCTGACGCAACCCCCACTGGTTGGGGCATTGCCACCACC |
| | | TGTCAGCTCCTTTCCGGGACTTTCGCTTTCCCCCTCCCTATTGCCACGGCGGAACTCA |
| | | TCGCCGCCTGCCTTGCCCGCTGCTGGACAGGGGCTCGGCTGTTGGGCACTGACAATTC |
| | | CGTGGTGTTGTCGGGGAAATCATCGTCCTTTCCTTGGCTGCTCGCCTGTGTTGCCACC |
| | | TGGATTCTGCGCGGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCATCGATACCGAGCGCTGCTC |
| | | GAGAGATCTACCTGTGCCTTCTAGTTGCCAGCCATCTGTTGTTTGCCCCTCCCCCGTG |
| | | CCTTCCTTGACCCTGGAAGGTGCCACTCCCACTGTCCTTTCCTAATAAAATGAGGAAA |
| | | TTGCATCGCATTGTCTGAGTAGGTGTCATTCTATTCTGGGGGGTGGGGTGGGGCAGGA |
| | | CAGCAAGGGGGAGGATTGGGAAGACAATAGCAGGCATGCTGGGGATGCGGTGGGCTCT |
| | | ATGG |

### EXAMPLES

### Example 1

The GALT null mouse is a genetic model of galactosemia. C57Bl/6J female mice were allowed to mate with infertile males to prepare them hormonally for implantation of a genetically modified blastocyst. The GALT gene in these blastocysts was trapped by replacing critical exons with an inactive but easily traceable sequence. The resulting mice were bred for homozygous trapping of the GALT gene. All GALT null mice have Type 1 galactosemia before birth, since they are bred from other GALT null mice. The neonatal timing of intervention at P9 is intended to target the period of disease onset before extended organ damage and represent a comparable stage of development to a young human pediatric patient population.

This animal model recapitulates many characteristics of galactosemia seen in humans, such as a lack of GALT enzyme activity and elevated Gal-1P levels in RBCs, lower fertility rates in females, motor deficit demonstrated via rotarod performance, and high mortality rate of pups (≥70%) if dams are fed a diet high in galactose (similar to increases in neonatal death seen in galactosemia patients if dietary restrictions are not implemented). See Table 2. Further, as seen in galactosemia patients, growth rates were slower in GALT null mice than in wild type counterparts (especially if fed galactose) (Tang et al 2014).

TABLE 2
Comparison Mouse Model Phenotypes to Human Phenotype

| Phenotype | Progression of Phenotype | Similarities to Human |
|---|---|---|
| Biochemical | Elevated galactosemia biomarkers | Biochemical changes, including |
| | from birth (Gal-1P, galactitol and | increased levels of Gal-1P, |
| | galactose) | galactitol and galactose |
| Growth Delay | Mild growth delay present from birth | Growth delay in patients |
| | until puberty (6 weeks old) | |
| Behavior | Motor deficit present in adult animals | Motor deficits in patients¹ |
| | and presence of cognitive deficit being | |
| | explored in ongoing PoC study² | |
| Fertility | Subfertility present post-puberty (from | Increased ovarian insufficiency in |
| | 6 weeks old) | female patients |

¹Presence of tremor and/or anxiety phenotype not reported in rodent model
²Cognitive deficits have not been previously characterized

The primary objectives were to evaluate the efficacy of AAV9-CAG-hGalt at lowering blood and tissue metabolites and to evaluate biodistribution of AAV9-CAG-hGalt in a mouse model of galactosemia. Two doses of AAV9-CAG-hGalt were examined. A total number of 39 animals (32 GALT null; 7 wild type) were administered AAV9-CAG-hGalt at two doses (3.74×10¹³ vg/kg and 1.15×10¹⁴ vg/kg) via IV dosing (tail vein) at Day P9. AAV9-CAG-hGalt—formulation buffer (vehicle)-treated mice and wild type mice served as the control arms. The endpoints of this study comprise improvement in GALT function (enzyme activity) (Table 3, below), attenuation of Type 1 galactosemia biomarkers (i.e., GALT, Gal-1P, galactitol, and galactose; evaluated using non-qualified test methods) and biodistribution in various organs that was measured at 4 and 12 weeks after treatment via GALT immunohistochemistry with PCR data pending.

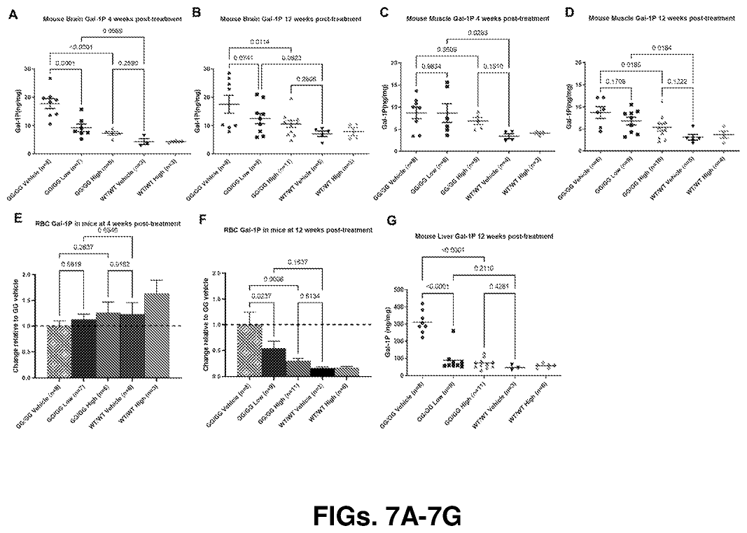

Evaluation of Gal-1P levels in GALT null mouse pups upon treatment with AAV9-CAG-hGalt has shown a significant reduction in the brain tissue at both 4 weeks (low dose: 48% and high dose: 59%) and 12 weeks (low dose: 28% and high dose: 40%) as compared to GALT null vehicle-treated mice (FIG. 7 A, B). Although no changes in skeletal muscle Gal-1P levels were observed at 4 weeks post-treatment at either dose (FIG. 7 C), a significant 38% reduction in skeletal muscle Gal-1P levels in high dosed AAV9-CAG-hGalt animals has been observed at 12 weeks post-treatment (FIG. 7 D). Similarly, no changes in RBC Gal-1P levels were observed at 4 weeks at either dose (FIG. 7 E), however a significant reduction in RBC Gal-1P levels in AAV9-CAG-hGalt animals (low dose: 45.8% and high dose 70.3%) was observed at 12 weeks post-treatment (FIG. 7 F). Liver showed highly significant reductions in Gal-1P at 12 weeks (72% and 77% reductions from vehicle for low and high doses, respectively) with 4 week data still pending.

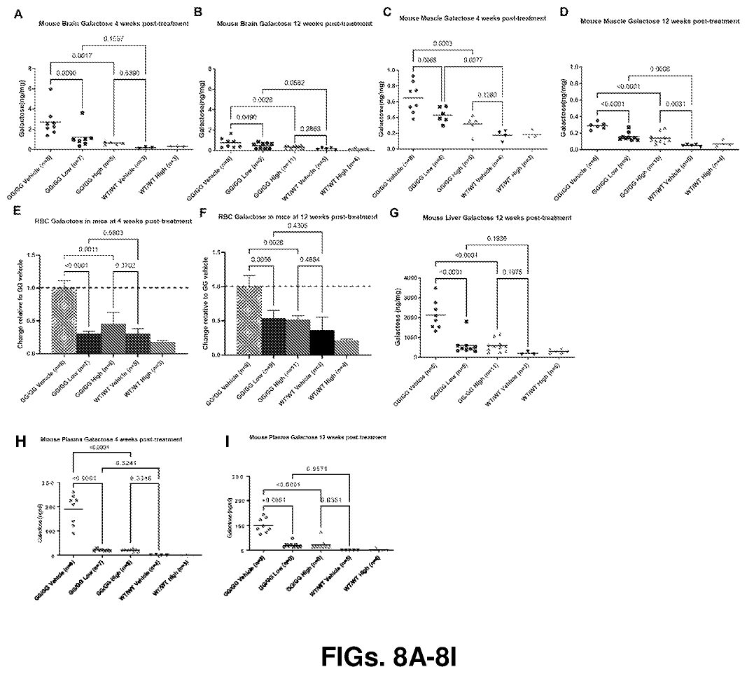

In line with the observed reduction in Gal-1P, significant reduction in galactose levels was observed in the brain tissue at both 4 weeks (low dose: 55% and high dose: 76%) and 12 weeks (low dose: 37% and high dose 56%) as compared to GALT null vehicle-treated mice (FIG. 8 A, B). Significant reduction in skeletal muscle galactose levels was also observed at both 4 weeks (low dose: 34% and high dose: 51%) and 12 weeks (low dose: 45% and high dose 52%) post-treatment (FIGS. 8 C, D). Galactose levels in RBC's were significantly reduced at both 4 weeks (low dose: 70% and high dose 54%) and at 12 weeks (low dose: 46.4% and high dose 48.8%) post-treatment (FIGS. 8 E, F). Galactose levels in plasma were significantly reduced at both 4 weeks (low dose: 86% and high dose 89%) and at 12 weeks (low dose: 80% and high dose 78%) post-treatment (FIGS. 8 H, I). Liver showed highly significant reductions in galactose at 12 weeks (72% and 73% reductions from vehicle for low and high doses, respectively) (FIG. 8G) with 4 week data still pending.

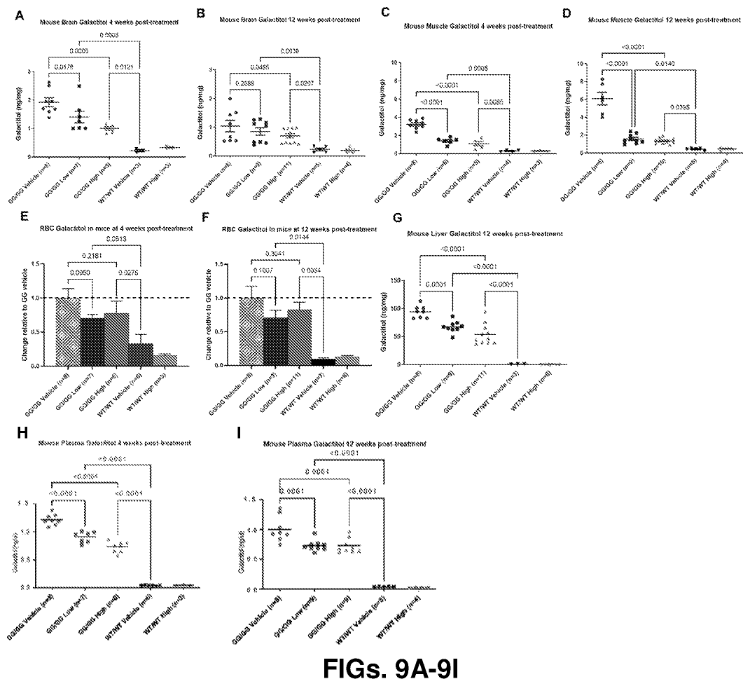

Significant reduction in galactitol levels was observed in the brain tissue at both 4 weeks (low dose: 27% and high dose: 48%) and 12 weeks (high dose 33%) as compared to GALT null vehicle-treated mice (FIGS. 9 A, B). Skeletal muscle galactitol levels were also significantly reduced at both 4 weeks (low dose 58% and high dose: 67%) and 12 weeks (low dose 74% and high dose 78%) post-dosing (FIGS. 9 C, D). Plasma galactitol levels were also significantly reduced at both 4 weeks (low dose 25% and high dose: 39%) and 12 weeks (low dose 27% and high dose 37%) post-dosing (FIGS. 9 H, I). However, no significant changes in RBC galactitol were observed at either 4 weeks or 12 weeks post-dosing (FIGS. 9 E, F). Liver showed highly significant reductions in galactitol at 12 weeks (28% and 57% reductions from vehicle for low and high doses, respectively) (FIG. 9G) with 4 week data still pending.

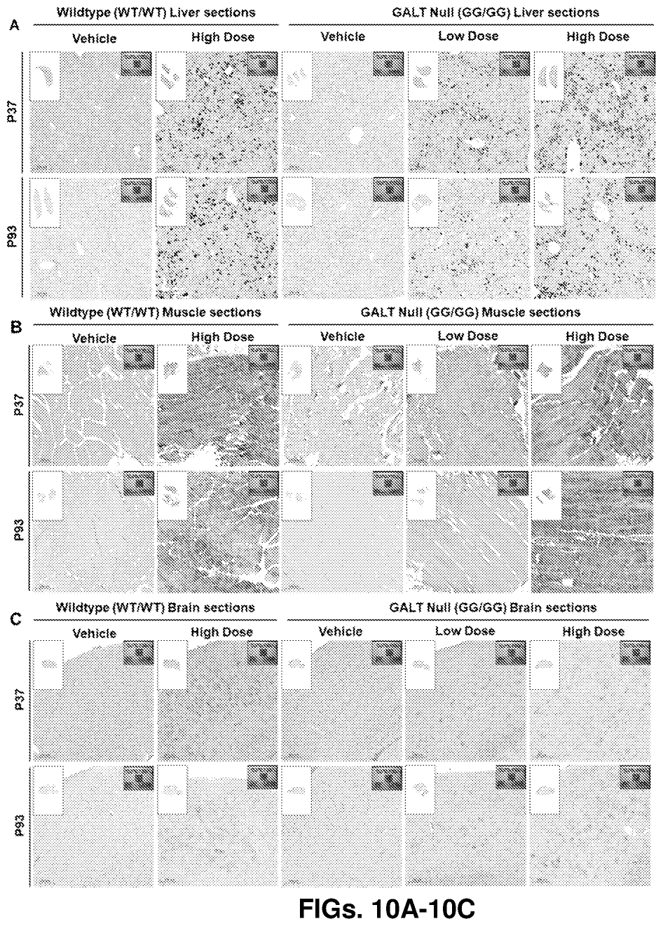

Assessment of GALT enzyme expression in multiple tissues by immunohistochemistry showed robust induction of GALT protein expression following AAV9-CAG-hGalt treatment in GALT null mice at P37 and P93 (4- and 12-weeks post-treatment with AAV9-CAG-hGalt, respectively), compared to the absence of GALT protein staining in vehicle treated GALT null animals (FIG. 10). Within the liver, GALT protein staining of AAV9-CAG-hGalt treated wild type and GALT null animals demonstrates increased GALT protein expression compared to respective vehicle treated controls (FIG. 10 A). Both low dose and high dose treatment of GALT null animals with AAV9-CAG-hGalt results in strong GALT protein expression at P37, with significant GALT staining persisting at P93. Within skeletal muscle, GALT protein staining shows robust induction of GALT protein expression following treatment of GALT null animals with AAV9-CAG-hGalt at P37, with robust GALT staining persisting at P93 (FIG. 10 B). Within the brain, GALT protein staining of GALT null mice demonstrates AAV9-CAG-hGalt-induced GALT protein expression at P37, which is sustained to P93 (FIG. 10 C). Higher levels of GALT protein staining are observed in the high dose treated animals, compared to the lower levels in low dose treated, demonstrating a dose dependent increase in AAV9-CAG-hGalt brain transduction. Transduction of neural and glial cells are observed throughout the brain at different efficiencies depending on structure, as exhibited by representative data in the cortex (FIG. 10 C).

Assessment of GALT enzyme activity (e.g., by quantifying the conversion of Gal-1P to UDP-gal via HPLC) in liver, brain and muscle showed robust induction of GALT enzyme activity following AAV9-CAG-hGalt treatment in GALT null mice at P93 (12-weeks post-treatment with AAV9-CAG-hGalt, respectively), compared to the absence of GALT enzyme activity in vehicle treated GALT null animals (Table 3). Within the liver, GALT enzyme activity of GALT null animals demonstrates increased GALT enzyme activity in both low dose (53.4%) and high dose (159%) treated animals compared to vehicle treated controls (1.3%). Within the brain, GALT enzyme activity of GALT null animals demonstrates increased GALT enzyme activity in both low dose (50%) and high dose (28%) treated animals compared to vehicle treated controls (2.6%). Within the muscle, GALT enzyme activity of GALT null animals demonstrates increased GALT enzyme activity in both low dose (147%) and high dose (177%) treated animals compared to vehicle treated controls (8%).

TABLE 3
Administration of AAV9-CAG-hGalt Increases GALT Activity in the Liver,
Brain, and Skeletal Muscle of GALT-null Mice at 12 Weeks Post-Treatment

| | % of Wild Type GALT Enzyme Activity | | | | |
|---|---|---|---|---|---|
| | (pmol UDPgal/mg protein/min) | | | | |
| | GG/GG | GG/GG | GG/GG | WT/WT | WT/WT |
| Tissue | Vehicle | Low | High | Vehicle | High |
| Liver | 1.3 ± 1.3% | 53.4 ± 18% | 159 ± 42% | 100 ± 13% | 178 ± 34% |
| Brain | 2.6 ± 2% | 50 ± 26% | 28 ± 10% | 100 ± 49% | 176 ± 44% |
| Muscle | 8 ± 5% | 147 ± 74% | 177 ± 65% | 100 ± 8% | 384 ± 33% |

A one-time intravenous delivery of AAV9-CAG-hGalt to mice results in robust transgene expression in key tissues throughout the body through the last time point examined (12 weeks post dosing. The presence of increased GALT enzyme in tissues accompanies metabolite reductions in those tissues as reductions in Gal-1P, galactose and galactitol were widely observed in AAV9-CAG-hGalt treated animals also through the 12 weeks. An approximately 3× difference between low and high dose did not result in a similar fold change in metabolite levels suggesting a similar potential prospect of benefit among the doses. Finally, the transduction of RBCs has been demonstrated elsewhere (Pasi et al 2020), though there is little expectation of sustained transgene expression in the non-nucleated cells. In the context of this experiment, findings suggest that changes in RBC metabolites are likely products of their environment and lifespan. These data suggest that while RBCs can be used to measure metabolites they are likely more indicative of changes in tissue galactose metabolism, particularly Gal-1P, instead of changes to galactose metabolism function within RBCs.

### Example 2

Previous work (Tang et al., 2014; Balakrishnan et al., 2020) documents that the—GalT gene-trapped mice to be used here demonstrate both relevant biochemical/metabolic markers of classic galactosemia and many of the long-term complications experienced by patients, including growth delay, motor impairment, and subfertility. Previous study (Balakrishnan et al., 2020) also documents the ability of intravenously administered GALT mRNA in restoration of hepatic GALT activity that led to a decrease in galactose-1 phosphate (Gal-1P) and plasma galactose in the mouse model.

The purpose of this study in GALT-deficient mice is to determine the effectiveness of AAV9-CAG-hGalt, an AAV9-based GALT gene therapy, to correct the disease-relevant phenotypes in the mouse model of Galactosemia. This study will be conducted using material manufactured with a clinical process (non-GMP) designed to enhance potency while reducing impurities at 200 L scale.

This work will be divided into three arms
i. Behavioral (Neuromuscular and Cognitive)
ii. Safety (Clinically relevant blood and tissue pathology)
iii. Fertility (Reproductive hormones and gestation)

Each arm will have a follow-up period of up to 6 months. In-life assessments with GALT activity measured in tissues at euthanasia and longitudinal blood and end point tissue levels of CG-associated metabolites: galactitol, Gal-1P, and galactose will be evaluated. Tissue histology and GALT expression in tissues defined by immunohistochemistry will also be followed in end point samples. CT scan for bone density and clinical chemistry will also be conducted in the same mouse model.

AAV9-CAG-hGalt is a suspension of an adeno-associated viral vector-based gene therapy for parenteral administration. It is a recombinant nonreplicating AAV9 vector containing a self-complementary transgene encoding for the human Galactose-1-phosphate uridylyltransferase (GALT) protein, under the control of a cytomegalovirus enhancer/chicken-β-actin hybrid promoter. AAV9-CAG-hGalt is supplied in sterile multi-use preservative-free cryovials at 3 separate concentrations. For the purposes of this study, the control article for AAV9-CAG-hGalt is supplied in sterile multi-use, preservative free vials. The solution filled in vials consists of the same excipient mixture utilized to formulate & fill the test article. Each test article vial contains sufficient material to dose a maximum of 2 mice

This proof-of-concept study will be conducted as a blinded study, where personnel will be blinded towards the treatment group and dosage. Table 4 below outlines the blinded test and control articles where the dose group will be omitted and will be labelled with a random blinded identifier, such as FRM-X representing “Formulation X”:

TABLE 4

| Test Article | | Nominal Fill | # of |
|---|---|---|---|
| Description | Excipient | Volume | Vials |
| Mouse (WT) | 10 mM Tris, 1 mM magnesium chloride (MgCl2), 150 mM | 63 μl | 23 |
| Vehicle | sodium chloride (NaCl) and 0.02% poloxamer 188, pH 8.0 | | |
| Mouse (WT) | 10 mM Tris, 1 mM magnesium chloride (MgCl2), 150 mM | 63 μl | 23 |
| High Dose | sodium chloride (NaCl) and 0.02% poloxamer 188, pH 8.0 | | |
| Mouse (GG) | 10 mM Tris, 1 mM magnesium chloride (MgCl12), 150 mM | 63 μl | 23 |
| Vehicle | sodium chloride (NaCl) and 0.02% poloxamer 188, pH 8.0 | | |
| Mouse (GG) | 10 mM Tris, 1 mM magnesium chloride (MgCl2), 150 mM | 63 μl | 23 |
| Low Dose | sodium chloride (NaCl) and 0.02% poloxamer 188, pH 8.0 | | |
| Mouse (GG) | 10 mM Tris, 1 mM magnesium chloride (MgCl2), 150 mM | 63 μl | 23 |
| Medium Dose | sodium chloride (NaCl) and 0.02% poloxamer 188, pH 8.0 | | |
| Mouse (GG) | 10 mM Tris, 1 mM magnesium chloride (MgCl2), 150 mM | 63 μl | 23 |
| High Dose | sodium chloride (NaCl) and 0.02% poloxamer 188, pH 8.0 | | |

The articles will be stored at ≤−60° C. Each vial will be thawed just before use at room temperature and article will be administered within a maximum of 2 hours post-thawing. Thawed vials will not be refrozen, and any unused material in opened vials will be discarded with biohazard waste.

Both behavioral and fertility cohorts are designed as dose-finding and efficacy studies. High, medium, and low doses, along with a vehicle control will be tested in all 3 study arms. All animals upon weaning will be placed on 2.5% galactose diet.

The studies will be conducted as blinded studies, where personnel at the study site involved in the performance of the studies (including treatment administration procedures and in-life and post-life assessments and analyses) will be blinded towards the treatment and dosage. The blinding will be lifted after completion of all in-life activities and post-life assessments. In addition to blinding, vehicle control cohorts will be included throughout the study to minimize the effects of confounding variables and compare against AAV9-CAG-hGalt treatment cohorts.

### Behavioral Study Arm

The full list of mice animal numbers for the behavioral arm, genotypes, treatment groups and sacrifice time points are outlined in Table 5 below:

TABLE 5

| Genotype | Treatment | 3 Month | 6 Month | Total |
|---|---|---|---|---|
| WT (GalT+) | Mouse WT - W | 10 | 10 | 20 |
| | Mouse WT - D | 10 | 10 | 20 |
| GG (GalT-null) | Mouse GG - T | 10 | 10 | 20 |
| | Mouse GG - C | 10 | 10 | 20 |
| | Mouse GG - B | 10 | 10 | 20 |
| | Mouse GG - A | 10 | 10 | 20 |

| | | | |
|---|---|---|---|
| Total | 60 | 60 | 120 |

n ≥ 3M and n ≥ 3F, will be designated for each time point.

The primary focus of this study will be expression of GALT and rescue of behavioral phenotypes. The study will test whether AAV9-CAG-hGalt reduces incidence and prevents neurological complications and cognitive deficits. To investigate the effect of AAV9-CAG-hGalt on behavioral improvements, each animal will receive one intravenous treatment of AAV9-CAG-hGalt at P9 (9 days after estimated time of birth). AAV9-CAG-hGalt-formulation buffer (vehicle/control article) treated mice will serve as the control arm. Three endpoints are planned for the behavioral study. The assessments may be performed+/−1 week.

### The Rotarod Test

The Rotarod test will be conducted to assess changes in motor/neuromuscular coordination and will be performed every month for a total of 6 assessments through 6 months of age. The rotarod assessment will be initiated at 1 month after IV administration of test or control article in the following order.

Acclimation: Subjects will be moved from home cage to the testing room at least 30 minutes before either training or testing to minimize effects of stress on behavior during testing

Training: ⋅Subjects in their home cage will be moved to the testing room and allowed to acclimatize for 15 minutes to minimize effects of stress on behavior during testing. To confirm that all subjects are able to walk forward on the rotating rod, subjects will undergo training the day before the first round of testing. Subjects will be placed on the rotarod rotating at a constant speed of 4 RPM for a minimum of 180 seconds. If a subject falls off the rotarod during the training trial, the subject will be placed back onto the rotarod. At the end of the training trial the subject will be returned to the home cage for testing on the next day.

Testing: The rotarod will be set to accelerate from 4 to 40 RPM over a total of 300 seconds (approximate increase of 1 RPM every 8 seconds), where the top speed of 40 RPM is reached at 300 seconds. The subjects will be placed on the rotarod with the starting speed of 4 RPM. If a subject falls off the rotarod prior to test start, the subject will be placed back on the rotarod. Once all subjects are placed onto the rotarod, the assay will be started and latency to fall and speed (RPM) at fall will be recorded. Subjects will be allowed to undergo one passive rotation (where they grip the rod and perform a full rotation without walking) and continue the test if they recover. A second passive rotation will be recorded as a failure, where latency to and speed at fall will be recorded. The subjects will then be returned to the home cage and the apparatus between trials. A total of 3 trials will be conducted, separated by 5-minute rest intervals. The average and peak latency to fall over the 3 trials will be calculated and plotted.

### The Inverted Screen Test

The inverted screen test, conducted to assess changed in motor strength/coordination, will be performed every month for a total of 6 assessments through 6 months of age. The inverted screen test will be initiated 1 month after IV administration of test or control article. The assessments may be performed+/−1 week.

Acclimation: Subjects will be moved from home cage to the testing room at least 30 minutes before either training or testing to minimize effects of stress on behavior during testing.

Testing: ⋅Subjects in their home cage will be moved to the testing room and allowed to acclimatize for 15 or 30 minutes to minimize effects of stress on behavior during testing. ⋅Subject will be placed in the center of the wire mesh screen and the screen rotated to an inverted position in a single motion with the mouse's head declining first. The testing and timer are started once the screen is completely inverted. ⋅Screen is steadily placed a minimum of 40 cm-50 cm above a surface to ensure subjects are incentivized to not jump off the inverted screen and grip as long as possible. The time is recorded when the mouse falls off from the mesh (latency to fall) onto a padded surface below.

### The Morris Water Maze Test

The Morris water maze test will be performed to assess improvements in spatial learning in GALT gene-trapped mice. Animals will be assessed at both 3 months and 6 months after IV administration of test or control article. The assessments may be performed+/−1 week.

Acclimation: ⋅Subjects will be moved from home cage to the testing room at least 30 minutes before either training or testing to minimize effects of stress on behavior during testing

Training: Phase 1: Cued swimming (2-3 trials, Day 1, platform 5 mm above water level). Subject will be lowered into the desired pre-set quadrant in the pool and the trial will be performed for 60 seconds each. Training will be repeated 2 times. Video capture will begin once the subject is lowered into the pool. If platform is found and mounted, subject will be left to sit for 20 seconds, then rescued, dried with towel, and transferred to heated cage. If platform is not found or mounted, subject will gently be guided to and onto platform with gloved hand, let to sit for 20 seconds, then rescued and dried with towel and transferred to heated cage. After transferring the mouse, an aquarium net will be used to remove any debris in between trials

Training: Phase 2: Non-cued swimming (2 trials, Day 2-4, platform 5 mm under water level). Subject will be lowered into the desired quadrant in the pool and trial will be performed for 60 seconds each. Trials will be repeated by randomizing mouse quadrant placement as shown in the Table 6 below.

TABLE 6
Non-Cued Swimming Quadrant Selection

| Phase 2: Quadrant order | Day 1 | Day 2 | Day 3 |
|---|---|---|---|
| First quadrant | Q4 | Q2 | Q1 |
| Second quadrant | Q2 | Q1 | Q4 |

Video capture will begin once the subject is lowered into the pool. If the platform is found and mounted, subject will be left to sit for 20 seconds, then rescued, dried with towel, and transferred to heated cage. If the platform is not found or mounted, subject will gently be guided to and onto platform with a gloved hand, let to sit for 20 seconds, then rescued and dried with towel and transferred to heated cage. After transferring the mouse, an aquarium net will be used to remove any debris in between trials.

Testing: Phase 3: Probe trial (1 trial, Day 5, platform removed). Subject will be lowered into random quadrant (1, 2, 4) in the pool and trial will be performed for 180 seconds each. Video capture will begin once the subject is lowered into the pool. After 180 seconds have elapsed, the subject will be rescued, dried with a towel, and transferred to a heated cage. A mouse, an aquarium net will be used to remove any debris in between trials.

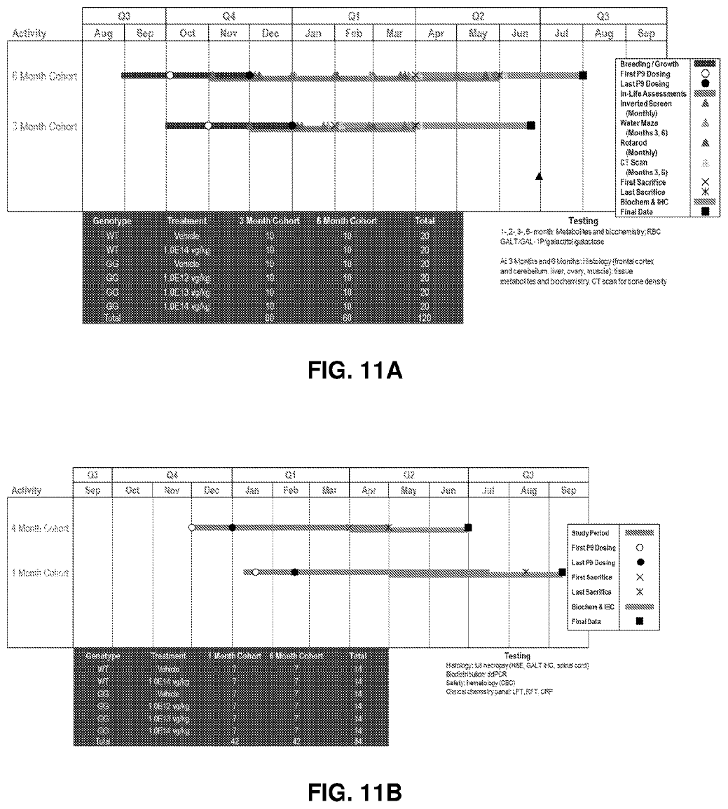

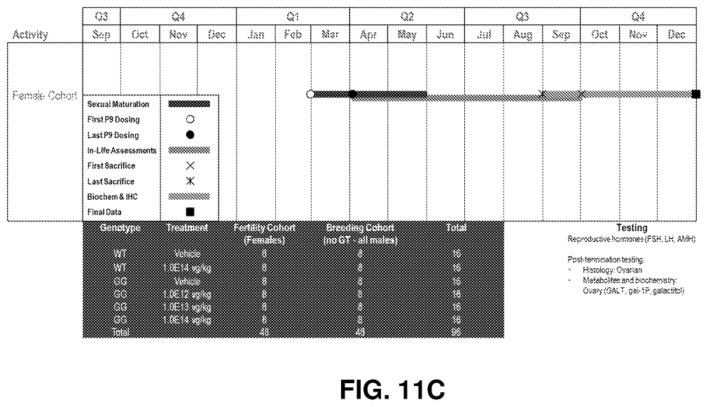

FIG. 11 is a chart showing the experimental design and data collection points. In-life assessment of metabolites for animals will be measured at 1-, 2-, 3-, and 6-months post-treatment for RBC GALT, RBC galactitol, RBC galactose, plasma galactose, plasma galactitol and plasma Gal-1P. 50% of the mice from all groups will be euthanized at 3 months after treatment, and the remaining 50% will be euthanized at 6 months after treatment. Post-termination collected tissue samples (brain, liver, ovary, muscle), collected at 3- and 6-month post-treatment, will be histologically assessed to confirm positive GALT expression: at each time point, n≥3. RBCs and collected tissues (brain, liver, ovary, muscle), will also be assessed for both ddPCR and metabolite biochemistry: at each time point, n≥6, 3M/3F, where available.

Growth of all the animals as assessed by body weights will be measured since birth. Mice body weights will be collected every 3 days starting at P9 till 1 month (P12, P15, P18, P21, P24, P27, P30, P33, P36, P39) and thereafter weekly i.e., P46, P53, P60, etc.) weight measurements. Under circumstances where weights are captured+/−1d than the specified date, then the weights will be grouped together (i.e., P21+/−1d). Computed tomography scans for bone density will be performed on the euthanized animals (n=6/group at 3- and 6-months).

### Fertility Study Arm

The full list of mice animal numbers for the fertility arm, genotypes, treatment groups and sacrifice time points are outlined in Table 7 below:

TABLE 7
Fertility Arm Parameters

| | | | Breeding- NO | |
|---|---|---|---|---|
| Genotype | Treatment | Fertility | Gene Therapy | Total |
| WT (GalT+) | Mouse WT - W | 8F | 8M | 16 |
| | Mouse WT - D | 8F | 8M | 16 |
| GG (GalT-null) | Mouse GG - T | 8F | 8M | 16 |
| | Mouse GG - C | 8F | 8M | 16 |
| | Mouse GG - B | 8F | 8M | 16 |
| | Mouse GG - A | 8F | 8M | 16 |

| | | | |
|---|---|---|---|
| Total | 48 | 48 | 96 |

The primary focus of this study will be reducing and rescuing ovarian failure/fertility in females. To investigate the effects, each animal will receive one IV treatment of test- or control article at P9 (9 days after estimated time of birth). The endpoints of this study will comprise changes in reproductive hormones (follicle-stimulating hormone, luteinizing hormone, and anti-Müllerian hormone) that will be assessed at the start and termination of the study.

Breeding setup will be followed using procedures described in Balakrishnan et al., 2019 (Section 1.7). Determination of the estrus cycle using cytology will be evaluated for the last two weeks prior to breeding and at the termination of the study to ensure that the blood and tissue samples collected for analysis are synchronized. Females will be moved to breeder cages and paired with one WT male (no treatment). Females will be then continuously mated from 11 weeks of age to 6 months of age or a minimum of three litters are achieved. Females in the breeder cage will be followed for visual or palpable signs of pregnancy. The planned study will allow determining the positive increases in pregnancy, time to pregnancy, litter size post-delivery, and pup survival. Post-termination collected ovarian tissue samples will be histologically assessed: n≥3. Collected ovary will also be assessed for attenuation in metabolite biochemistry: n≥5.

### Safety Dose Study Arm

The full list of mice animal numbers for the safety arm, genotypes, treatment groups and sacrifice time points are outlined in Table 8 below:

TABLE 8
Safety Arm Parameters

| Genotype | Treatment | T30 | T180 | Total |
|---|---|---|---|---|
| WT (GalT+) | Mouse WT - W | 7 | 7 | 14 |
| | Mouse WT - D | 7 | 7 | 14 |
| GG (GalT-null) | Mouse GG - T | 7 | 7 | 14 |
| | Mouse GG - C | 7 | 7 | 14 |
| | Mouse GG - B | 7 | 7 | 14 |
| | Mouse GG - A | 7 | 7 | 14 |

| | | | |
|---|---|---|---|
| Total | 42 | 42 | 84 |

n ≥ 3M and n ≥ 3F, will be designated for each time point.

The primary focus of this study arm will be to determine the preclinical safety profile in a preclinical pharmacology model of galactosemia. To investigate the effects, each animal will receive one IV treatment of test- or control article at Day P9. Animals will be euthanized at approximately 30 days and approximately 180 days after treatment. Euthanasia may be performed up to +/−6 days to ensure overnight shipment and delivery of collected samples for clinical chemistry. The endpoints of this study comprises full necropsy and histology, and biodistribution on a comprehensive list of organs (listed under tissue collection table). Safety of AAV9-CAG-hGalt will also be assessed by hematology and clinical chemistry panels.

### Enrollment

Entire litters will be enrolled because the breeding pairs are kept in single cage, and they are from homozygous parents. There is no planned cross fostering. Not all the animals from the same litter will receive the same treatments. Using procedures described in Balakrishnan et al., 2019 (Section 1.7), individual pups from the same litter will be identified and differentiated. Pups will be randomly assigned within a litter to a dose level by study personnel. The total number of pups to be enrolled across various arms is provided in Table 9.

TABLE 9
Mouse Enrollment

| Genotype | Treatment | Total | Grand total |
|---|---|---|---|
| WT | Behavioral | 40 | 100 |
| | Fertility | 32 | |
| | Safety | 28 | |
| GG | Behavioral | 80 | 200 |
| | Fertility | 64 | |
| | Safety | 56 | |

| | |
|---|---|
| Total | 300 |

To ensure the correct volume of AAV vector is administered, all pups will be weighed, and will be injected with the required dose of 5 μL per g bodyweight. The mice pups that appear to be sick will not be injected as part of the study. The study will be conducted as a blinded study.

### Endpoints

For unscheduled sacs, section 5.5 will be followed and collect organs listed under tissue collection table.

For scheduled necropsy in the behavioral and fertility arm, mice will be randomly assigned to outcome measures. Animals designated for metabolite analysis will be deeply anesthetized and blood collected via transcardiac puncture. The collected whole blood will be separated into plasma and RBC by centrifugation. Plasma will be aliquoted for metabolite analysis (described below) and reproductive hormone analysis (females only). At each time point, n≥6 (3M/3F, where available for behavioral arm) will be designated for metabolite/biochemical analysis and GALT activity within tissues, plasma and RBCs.

The remaining animals in the group (n≥3, where available) will be assigned to histology, GALT IHC and ddPCR. For scheduled necropsy in the fertility cohort, animals will additionally undergo transcardiac perfusion with PBS following completion of blood collection via transcardiac puncture. For the fertility arm, ovarian tissue samples will be histologically assessed: n≥3. Collected ovary will also be assessed for attenuation in metabolite biochemistry: n≥5. For scheduled necropsy in the safety cohort, animals will be deeply anesthetized, and blood collected via transcardiac puncture for complete blood count and clinical chemistry.

Additionally, at scheduled necropsy blood samples will be taken from GG and WT “Safety” treatment groups for hematology and clinical chemistry assessments at IDEXX Bioanalytics (Sacramento, CA) as described below.

Comprehensive CBC with reticulocyte HGB will be analyzed. The endpoints are: White Blood Cells (WBC), Neutrophils (% and absolute), Band (% and absolute), Lymphocytes (% and absolute), Monocytes (% and absolute), Eosinophils (% and absolute), Basophils (% and absolute), Red Blood Cells (RBC), Hematocrit (HCT), Total Hemoglobin (HGB), Mean Corpuscular Volume (MCV), Mean Corpuscular Hemoglobin Concentration (MCHC), Mean Corpuscular Hemoglobin (MCH), Reticulocyte Count (% and absolute), Reticulocyte Hemoglobin Content, Platelet Count, and Platelet Estimate.

The clinical chemistry endpoints are: Alanine Aminotransferase (ALT), Albumin (ALB), Albumin: Globulin Ratio, Alkaline Phosphatase (ALP), Aspartate Aminotransferase (AST), Bicarbonate, Bilirubin (Conjugated), Bilirubin (Unconjugated), Total Bilirubin (TBIL), Blood Urea Nitrogen (BUN), BUN: Creatinine Ratio, Calcium (Ca), Chloride (Cl), Cholesterol (CHOL), Creatine Kinase (CK), Creatinine (CREA), Globulin (GLB), Glucose (GLU), Phosphorous (PHOS), Potassium (K), Sodium (Na), Sodium:Potassium Ratio, and Total Protein (TP).

For all the study arms, samples of the tissues will be collected and snap-frozen or placed into 10% neutral buffered formalin for a minimum of 48 hours and up to 72 hours. Tissues harvested for fixation will be collected in histology cassettes and placed into 125 ml screw-cap containers with sufficient 10% neutral buffered formalin to completely submerge the cassette. Fixed tissues will be sent for histological processing, GALT IHC, H&E staining and anatomic pathology. Frozen tissues will be cut into three aliquots approximately 25 mg each then placed into three round bottom 2 ml tubes. The tubes will be labeled with the animal ID and tissue. The tubes will be snap-frozen in liquid nitrogen then maintained at ≤−60° C. until analysis.

Mice body weights will be collected every 3 days starting at P9 until 1 month (P12, P15, P18, P21, P24, P27, P30, P33, P36, P39) and thereafter weekly i.e., P46, P53, P60, etc.) weight measurements.

GALT activity analyses in selected tissues (RBC/Brain/Liver/Ovary/Muscle/Plasma) will be performed using LCMS-MS.

GALT metabolite (galactose, galactitol, Gal-1P) analyses in selected tissues (RBC/Brain/Liver/Ovary/Muscle/Plasma) will be performed using LC-MS/MS. Each sample will be analyzed once, and the results will be treated as single data point. Samples will not be pooled for analysis.

Samples will be immersed and fixed in 10% neutral buffered formalin for a minimum of 48 hours and up to 72 hours at room temperature, then transferred to 70% ethanol and stored at room temperature until shipping. Following fixation, tissues will be shipped to Invicro for histological processing, H&E, and GALT immunohistochemistry.

Biodistribution of transgene DNA and RNA will be assessed by ddPCR on frozen tissues. All frozen tissue samples for Vector DNA and RNA ddPCR analysis will be shipped in batches on dry ice to Bioagylitix. Retain samples will be stored until the end of the project at ≤−60° C. Upon completion of the study.

Reproductive hormones (AMH, LH, FSH) analysis of plasma samples from the fertility arm will be run using ELISA kits.

CT scans of selected animals (n=6 per group) will be conducted at the Preclinical Imaging Core Facility at the University of Utah.

≥0.200 mL of whole blood will be collected for hematology assessment (LTT-MJNJ Top tube 0.5 ml), mixed end over end and stored at approximately 4° C. For clinical chemistry, ≥0.5 mL of whole blood will be added to serum collection tubes (SST-MJNJ Gold Top tube 0.8 ml), then gently inverted 5-10× times post collection, and samples allowed to clot at room temperature for 15-20 minutes, centrifuged at 2,500 rpm for 10 minutes, with off clot serum removed and combined into a new non-additive sample transfer tube (yielding >200 μL of serum), and stored at approximately 4° C.

The full list of organs to be collected for the safety arm and unscheduled deaths are outlined in the Table 10 below:

TABLE 10
Safety Arm Parameters

| Tissue | Frozen | Fixed |
|---|---|---|
| Adrenal gland | | X |
| Aorta | | X |
| Brain* | X | X |
| Cecum | | X |
| Colon | | X |
| Duodenum | | X |
| Epididymus | | X |
| Esophagus | | X |
| Eye | | X |
| Femur | | X |
| Gall bladder | | X |
| Heart | X | X |
| Ileum | | X |
| Jejunum | | X |
| Kidney | X | X |
| Liver | X | X |
| Lung | X | X |
| Lymph node | | X |
| Mammary gland | | X |
| Ovary | X | X |
| Pancreas | X | X |
| Parathyroid gland | | X |
| Pituitary | | X |
| Salivary gland | | X |
| Sciatic nerve | X | X |
| Seminal vesicle | | X |
| Skeletal muscle | X | X |
| Skin | | X |
| Spinal Cord | X | X |
| Spleen | X | X |
| Stomach | | X |
| Testis | X | X |
| Thymus | | X |
| Thyroid gland | | X |
| Trachea | | X |
| Urinary bladder | | X |
| Uterus | | X |
| Vagina | | X |

*Frozen brain sections will be taken from the left hemisphere. The left hemisphere will be divided into three sections. The most rostral section will go for metabolite analysis, the second and third most rostral sections will go for vector genome and transgene mRNA analyses, respectively. The right hemisphere will be fixed.

### Example 3

A study was conducted to assess the efficacy of AAV 2/9 expressing human GALT gene (AAV9-CAG-hGalt) at reducing cataracts in a rat model of classic galactosemia and correlate cataracts with tissue levels of GALT activity and both tissue and blood levels of CG-associated metabolites: galactitol, galactose-1-phosphate (Gal-1P), and galactose.

The GALT null rat is a genetic model of Type 1 galactosemia. Using clusters of regularly interspaced short palindromic repeats (CRISPR) gene editing, a 2-base pair insertion mutation (GaltM3) was introduced into exon 6 of the Galt locus in Sprague Dawley rats. Rats with homozygous GaltM3 mutations were then bred to produce the GALT null rat.

Absent treatment, GaltM3 homozygotes lack GALT enzyme activity at all ages. Pups are also born with lower birth weights and remain smaller than their wild type or heterozygous counterparts until puberty, at which point, similar to humans with Type 1 galactosemia they “catch up”. This rat genetic model has a 100% penetrance rate in terms of absent GALT activity and abnormal accumulation of galactose metabolites, so all GALT null rats have the basis for disease before birth similar to humans. The neonatal timing of intervention at P2 for this study is intended to target the period of disease onset before extended organ damage and represent a comparable stage of development to a young human pediatric patient population. GALT null rats also mimic characteristics of galactosemia seen in humans, such as the mild growth delay mentioned above, cataracts, and motor deficit and cognitive impairment, which are detected in mature animals. As in humans, these phenotypic outcomes show reduced penetrance and variable expressivity among individual rats but are clearly present when comparing cohorts of animals. Within 24 hours of birth, birthweights and levels of toxic metabolites associated with Type 1 galactosemia are present in GALT null rat pups (Rasmussen et al 2020).

An overview of the rat model and its relevance to the human presentation is provided in Table 11.

TABLE 11
Comparison of Rat Model Phenotype to Human Phenotype

| Phenotype | Progression of Phenotype | Similarities to Human |
|---|---|---|
| Biochemical | Elevated galactosemia biomarkers from birth | Biochemical changes, including increased |
| | (Gal-1P, galactitol and galactose) | levels of Gal-1P, galactitol and galactose |
| Growth Delay | Mild growth delay present from birth until | Pre-pubertal growth delay in patients |
| | puberty (6 weeks old) | |
| Behavior | Motor and cognitive deficits present in adult | Motor and cognitive deficits in patients¹ |
| | animals | |
| Cataracts | Bilateral cataracts present upon opening of | Increased occurrence of cataracts in patients |
| | eyes (at 2 weeks old) | exposed to milk |

The primary objectives of this study were to evaluate the efficacy of AAV9-CAG-hGalt at lowering blood and tissue metabolites, determine biodistribution and GALT enzymatic activity levels in tissues and assess end-organ damage in the lens of a rat model of galactosemia. GALT null mice have low GALT activity and high levels of galactose and its metabolites (galactose, galactitol and Gal-1P) in blood, liver, and brain. Mirroring human patients with galactosemia, GALT null rats additionally develop cataracts. Notably, the appearance of metabolic perturbation at birth and cataracts established by age P17 in the rat model supports early intervention to target this critical period of disease onset. Thus, P2 rats were dosed to target the period of disease after recognition of defective galactose metabolism but prior to onset of extended organ damage; thereby representing a comparable stage of development to a young human pediatric patient population. Furthermore, rats at P2 provide an early window after disease onset to perform IV dosing.

AAV9-CAG-hGalt was administered to wildtype and GALT null rats via IV dosing in tail vein at Day P2 using 2 doses (3.82×10¹³ vg/kg and 1.16×10¹⁴ vg/kg). AAV9-CAG-hGalt-formulation buffer (vehicle)-treated rats served as the control arm. The endpoints of this study comprise biodistribution in selected organs that were measured at 14 days and 35 days after treatment via GALT IHC (described below) and ddPCR (data is pending), increase in GALT enzyme activity in tissues, attenuation of Type 1 galactosemia biomarkers (i.e., GALT, Gal-1P, galactitol, galactose; assessed using non-qualified test methods), and assessment of cataract rate and severity. Automated ddPCR methods are known in the art and include, for example, BioRad's droplet digital PCR System.

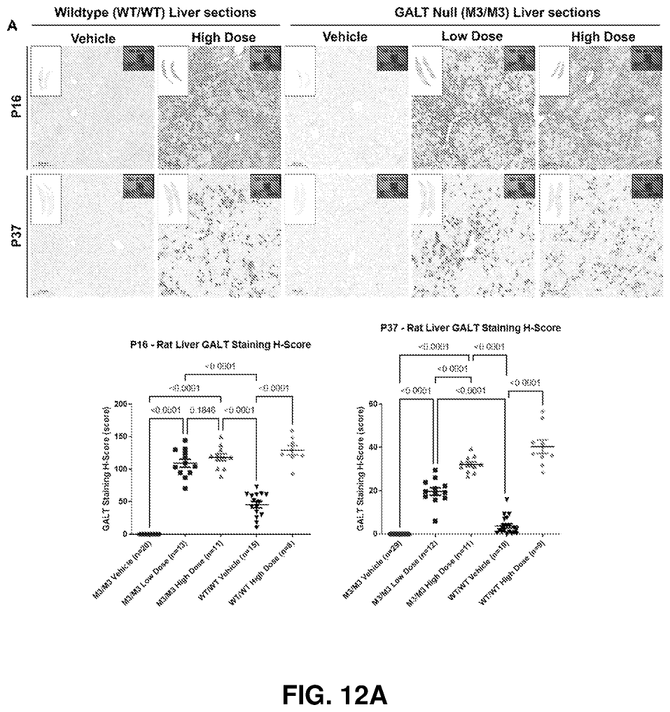

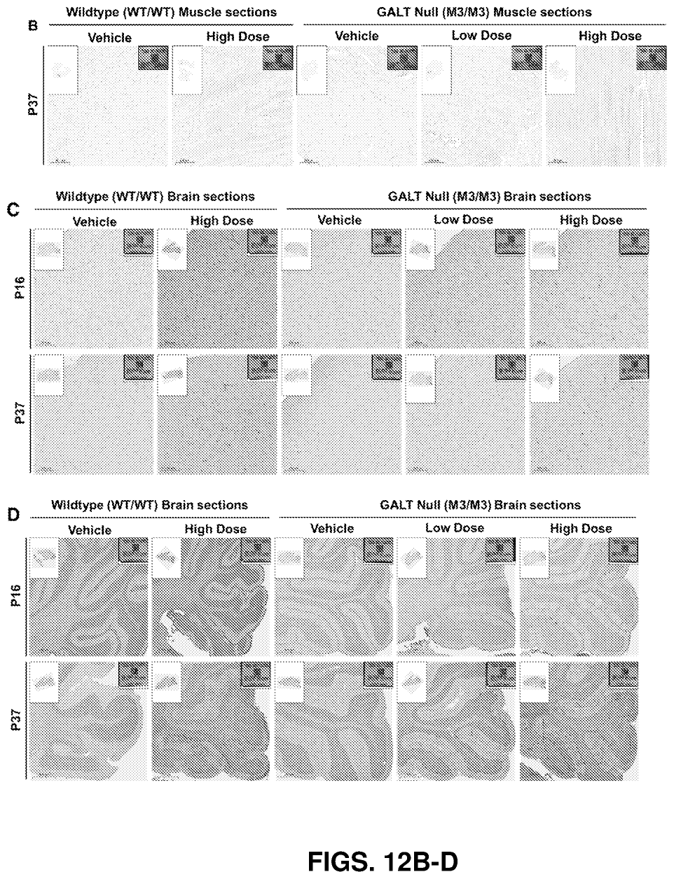

Assessment of GALT enzyme expression in multiple tissues by immunohistochemistry showed robust induction of GALT protein expression in GALT null rats P16 and P37 of age (14- and 35-days post-treatment with AAV9-CAG-hGalt, respectively), compared to the absence of GALT protein staining in vehicle treated GALT null animals (FIG. 12). Within the liver, anti-GALT immunohistochemistry (IHC) of AAV9-CAG-hGalt treated wildtype and GALT null animals demonstrated increased GALT protein expression compared to respective vehicle treated controls (FIG. 12A). Both low dose and high dose treatment of GALT null animals with AAV9-CAG-hGalt resulted in strong GALT protein expression at P16, with diminished but significant (as measured via H-Score) GALT staining persisting at P37. Interestingly, vehicle treated WT livers also saw a decrease in GALT immunoreactivity over the course of the study. Within skeletal muscle, GALT IHC shows induction of GALT protein expression following treatment of GALT null animals with AAV9-CAG-hGalt at P37 (FIG. 12B). Skeletal muscle at P16 was not examined. Within the brain, GALT IHC demonstrated AAV9-CAG-hGalt-induced GALT protein expression throughout the brain at P16 and persisting to P37, demonstrated by representative data in the cortex and cerebellum (FIG. 12 C and D). Higher levels of GALT protein staining were observed in the high dose treated animals, compared to the low dose treated animals, demonstrating a dose dependent increase in AAV9-CAG-hGalt brain transduction. Co-staining of the GALT protein in brown with the astrocytic glial cell marker GFAP (Glial Fibrillary Acid Protein) in purple demonstrated robust transduction of both neural and glial cells in the cortex (FIG. 12D). A dose dependent increase in Purkinje neuron transduction throughout the cerebellum was also observed following AAV9-CAG-hGalt administration (FIG. 12D). Ovaries from vehicle and AAV9-CAG-hGalt treated null rats were negative for GALT immunoreactivity (not shown) while signal was observed in wild type tissue.

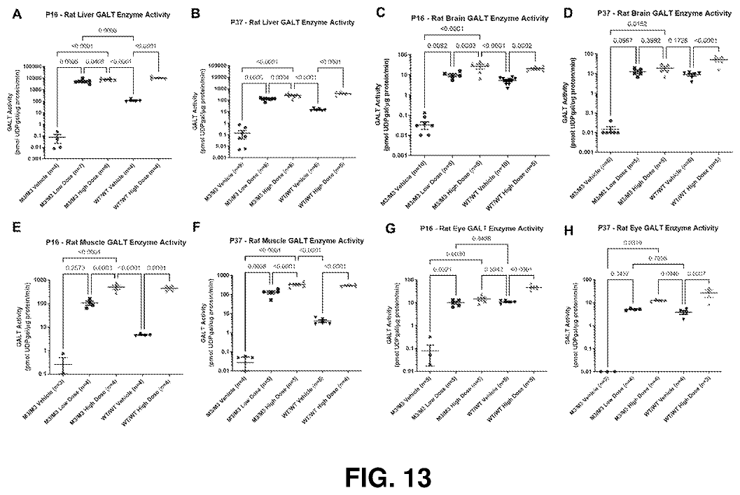

Increased GALT immunoreactivity in tissues corresponded to increased enzymatic activity as well. GALT enzyme activity data as measured by quantifying the conversion of Gal-1P to UDP-gal via HPLC in examined tissues (FIG. 13) at both P16 and P37 shows a highly significant increase as compared to GALT null rats. In the liver (FIG. 13 A,B) GALT enzyme activity was significantly increased 14 days post-treatment (low dose: 47-fold increase over wildtype and high dose: 70-fold increase over wildtype), with high levels maintained 35 days post-treatment (low dose: 6.4-fold increase over wildtype and high dose: 13.7-fold increase over wildtype). GALT enzyme activity was also significantly increased in brain tissue (FIG. 13 C,D) at 14 days post-treatment (low dose: 1.9-fold increase and high dose: 5.1-fold over wild type), which was sustained at 35 days post-treatment (low dose: 1.4-fold increase over wildtype and high dose: 2.1-fold increase over wildtype). Similarly, skeletal muscle tissue also demonstrated significant increase in GALT enzyme activity (FIG. 13 E,F) at 14 days post-treatment (low dose: 23-fold increase over wildtype and high dose: 108-fold increase over wildtype), which was sustained at 35 days post-treatment (low dose: 48.5-fold increase over wildtype and high dose: 84-fold increase over wildtype). Finally, GALT enzyme activity reached approximately wildtype levels within the eyes of AAV9-CAG-hGalt dosed animals at both 14 and 35 days post injection (FIG. 13 G,H).

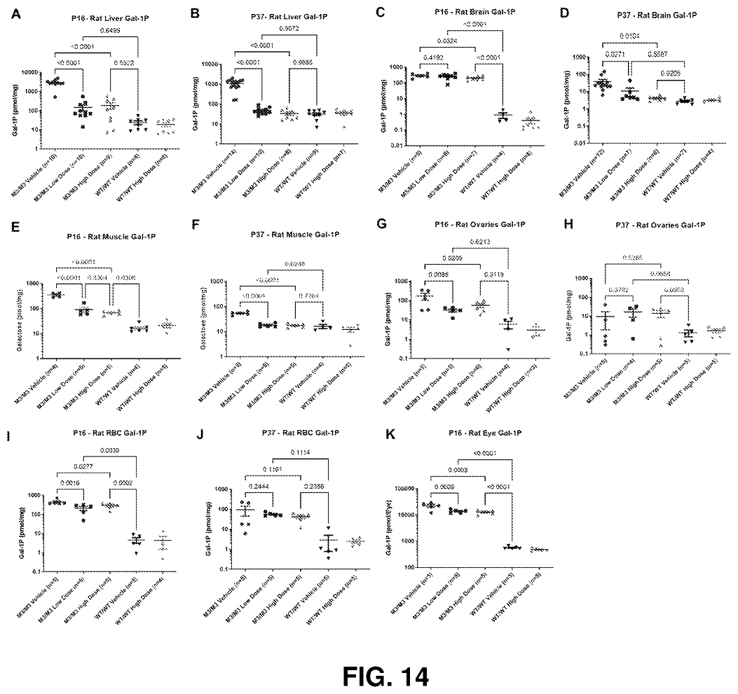

The positive expression of GALT confirmed biochemically by GALT enzyme activity assay (liver, brain, skeletal muscle and eye), and histologically (liver, muscle and brain) could subsequently lead to the reduction of toxic metabolic drivers (galactose, galactitol, and Gal-1P) of galactosemia. To test this hypothesis, metabolites were examined in tissues of AAV9-CAG-hGalt and vehicle treated M3 and WT rats. Levels in GALT null rat pups (M3) upon treatment with AAV9-CAG-hGalt show a significant reduction in Gal-1P within liver tissue at P16 (low dose: 95% and high dose: 93%). The lowering in liver Gal-1P was maintained through at least P37 (low dose: 95% and high dose: 97%) as compared to GALT null vehicle-treated rats (FIG. 14 A,B). No changes in brain Gal-1P levels were observed at P16 following treatment with low dose AAV9-CAG-hGalt although, high dose led to a significant reduction in brain Gal-1P at P16 (FIG. 14 C). Furthermore, a significant reduction in brain Gal-1P levels, in AAV9-CAG-hGalt treated rats, at both low and high dose (low dose: 72% and high dose: 89%) was observed at P37 (FIG. 14 D). Interestingly, despite a lack of overt GALT expression in AAV9-CAG-hGalt treated ovaries by IHC, there were 82% and 67% reductions in Gal-1P in ovaries from AAV9-CAG-hGalt low and high dose animals, respectively, from vehicle levels at P16 (FIG. 14 G). At P37, Gal-1P levels reduced in ovaries of vehicle treated null animals (FIG. 14 H) relative to P16 vehicle without a corresponding lowering of Gal-1P in vector treated null animals. A significant reduction in RBC Gal-1P levels (low dose: 52% and high dose 34%) was observed in vector treated null rats at P16 (FIG. 14 I). However, no significant reduction in RBC Gal-1P levels were seen at P37 null rats (FIG. 14 J). Notably Gal-1P levels in the eyes of vector treated null rats was significantly reduced at P16 for both doses of AAV9-CAG-hGalt (low dose: 37% and high dose 42%). Gal-1P data from eyes are pending for the P37 time point.

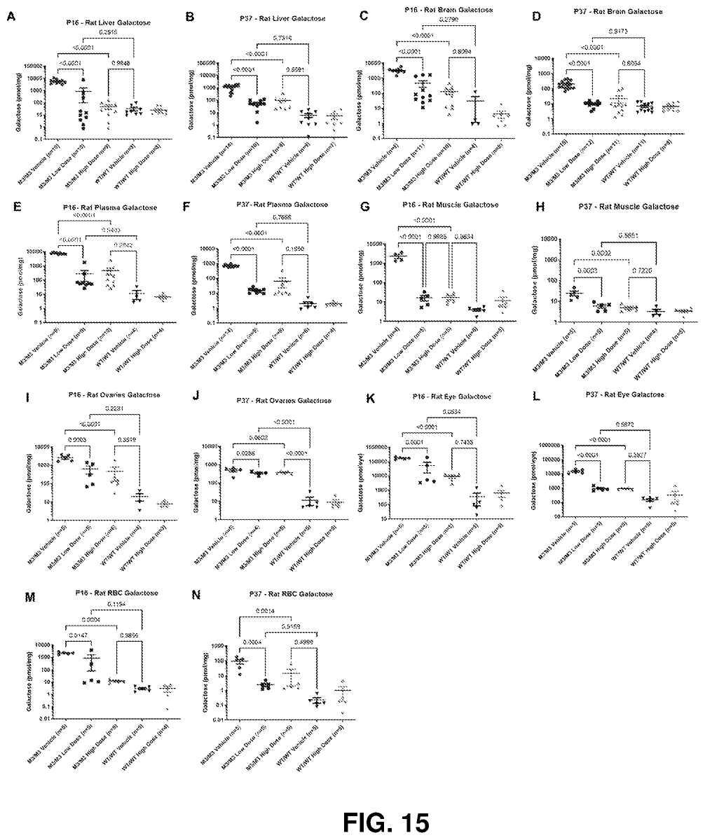

Galactose levels were also evaluated to determine whether positive observation in GALT expression corresponded to a reduction in galactose. The data (FIG. 15) show that while vehicle treated GALT null pups had high liver (FIG. 15 A, B), brain (FIG. 15 C, D), plasma (FIG. 15 E, F), muscle (FIG. 15 G, H), ovary (FIG. 15 I, J) eye (FIG. 15 K, L), and RBC galactose levels, nearly all tissues from animals treated with AAV9-CAG-hGalt showed significantly reduced levels of galactose at both P16 and P37, while ovary from null animals that received high dose AAV9-CAG-hGalt trended towards significance (p=0.0602).

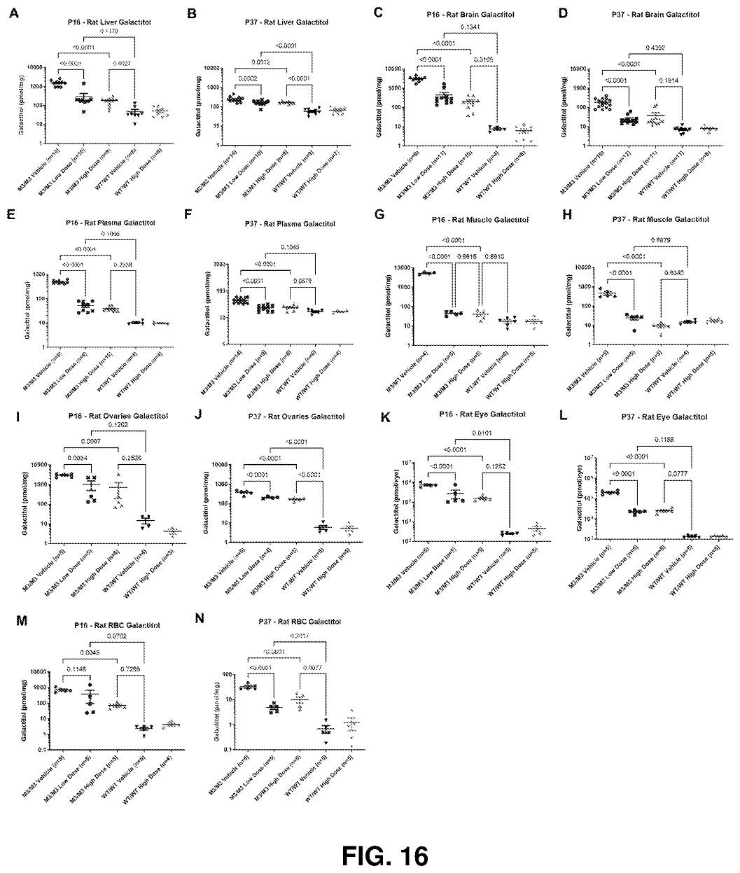

The effect of AAV9-CAG-hGalt on galactitol, a key metabolite responsible for cataract formation in galactosemia patients, was also evaluated. Similar to observed reduction in Gal-1P and galactose levels, galactitol levels were also reduced upon treatment with AAV9-CAG-hGalt. Galactitol levels (FIG. 16) showed statistically significant reductions in all tissues examined (liver, brain, plasma, muscle, ovary and eyes) from null animals that received AAV9-CAG-hGalt treatment compared to vehicle at both timepoints examined (14 and 35 days) post-injection. Significant reductions in RBC galactitol levels were observed in high dose at 14 days post-treatment and in both low and high dose at 14 and 35 days post-treatment with AAV9-CAG-hGalt (FIG. 16).

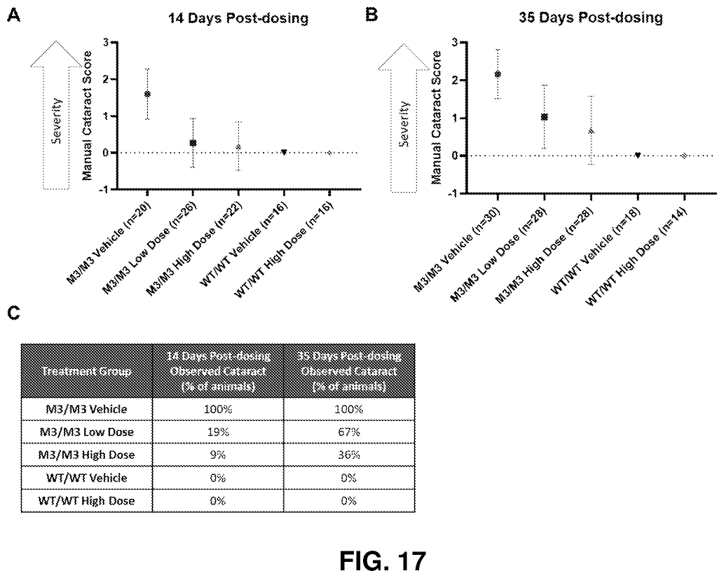

Assessment of GALT null rat pups at P16 and P37 that were treated with AAV9-CAG-hGalt presented considerably lower severity (FIG. 17 A,B) and incidence of cataracts (FIG. 17 C).

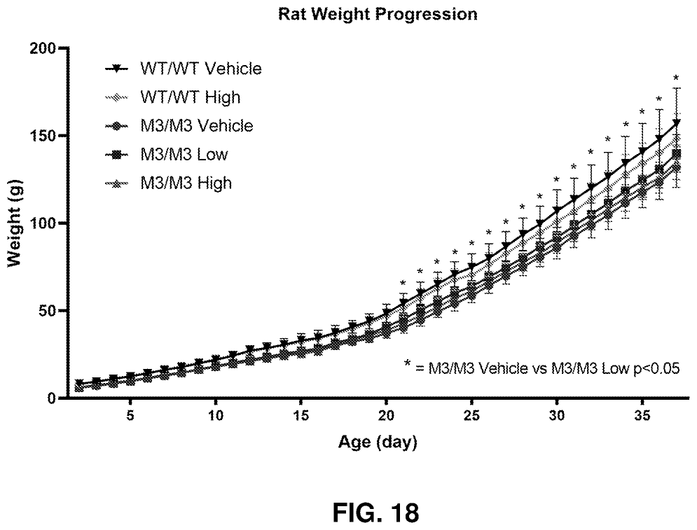

Furthermore, an overall increase in weights of the AAV9-CAG-hGalt treated GALT null rat pups has also been observed (FIG. 18).

Systemic delivery of AAV9-CAG-hGalt in GALT null rats resulted in widespread transgene expression in tissues that are known to be transduced by AAV9. Importantly, expression levels remained robust through the timepoints examined in the experiment with staining intensity and enzymatic activity at or greater than wild type levels. Increased GALT enzyme corresponded to a lowering of galactosemia associated metabolites in AAV9-CAG-hGalt treated null rats. Notably, metabolite lowering in eye was associated with reduced cataract incidence and severity within null rats that received AAV9-CAG-hGalt. Vehicle metabolite levels spontaneously decreased with increasing age. The reduction was likely due to multiple factors including the transition from mother's milk to low galactose chow also likely reduced endogenous production of galactose with age and the upregulation of alternative galactose metabolic pathways. Overall, these data show AAV9-CAG-hGalt treatment is highly impactful at lowering galactosemia related metabolites within well and poorly transduced tissues and resulted in improvements in rate and severity of cataracts.

TABLE 16
Rat Pre-PoC: Data at 14 days and 35 days post-treatment

| | Table 16 | | | | |
|---|---|---|---|---|---|
| | % of Wild Type GALT enzyme activity (pmol UDPgal/ug protein/min) | | | | |
| | M3/M3 | M3/M3 | M3/M3 | WT/WT | WT/WT |
| | Vehicle | Low | High | Vehicle | High |
| Liver P16 | 1.5% | 4,968% | 7,676% | 100% | 12,291% |
| Liver P37 | 0% | 866% | 1,733% | 100% | |
| Brain P16 | 1% | 188% | 509% | 100% | 387% |
| Brain P37 | 0% | 142% | 215% | 100% | 573% |
| Skeletal Muscle P16 | 6% | 2,285% | 10,808% | 100% | 9,555% |
| Skeletal Muscle P37 | 1% | 3,198% | 8,150% | 100% | 7,196% |
| Eye P16 | 1% | 92% | 135% | 100% | 429% |
| Eye P37 | 0% | 132% | 308% | 100% | 596% |

### Example 4

The purpose of the proposed work is to conduct a proof-of-concept study using material made at scale with a clinical grade process in GALT-null rats to assess efficacy of AAV9-CAG-hGalt at three dose levels in preventing long-term cataract development and attenuation of other phenotypes including pre-pubertal growth delay, visceral neuromuscular deficit via grip strength measurements, and neurological or socioemotional deficits via open field and forced swim assessments. The aim is to correlate these in-life assessments with GALT activity measured in tissues at euthanasia and longitudinal blood and end point tissue levels of CG-associated metabolites: galactitol, Gal-1P, and galactose. Tissue histology and GALT expression in tissues defined by immunohistochemistry will also be followed in end point samples.

AAV9-CAG-hGalt is a suspension of an adeno-associated viral vector-based gene therapy for parenteral administration. It is a recombinant nonreplicating AAV9 vector containing a self-complementary transgene encoding for the human Galactose-1-phosphate uridylyltransferase (GALT) protein, under the control of a cytomegalovirus enhancer/chicken-β-actin hybrid promoter. For the purpose of this study, AAV9-CAG-hGalt was produced and will be supplied in sterile, multi-use, preservative-free cryovials at 3 separate concentrations. The vehicle control is also supplied in sterile, multi-use, preservative-free vials. The solution filled in the vehicle control vials consists of the same excipient mixture utilized to formulate & fill AAV9-CAG-hGalt. Each vial contains sufficient material to dose up to 4 neonatal rat pups.

This study will be conducted as a blinded study, where personnel will be blinded towards the treatment group and dosage. Table 12 below outlines the blinded test and control articles where the dose group will be omitted and will be labelled with a random blinded identifier, such as FRM-X representing “Formulation X”:

TABLE 12

| Test and Control | | Nominal | |
|---|---|---|---|
| Article | | Fill | # of |
| Description | Excipient | Volume | Vials |
| Rat WT Vehicle | 10 mM Tris, 1 mM magnesium chloride (MgCl₂), 150 mM | 259 μL | 6 |
| | sodium chloride (NaCl) and 0.02% poloxamer 188, pH 8.0 | | |
| Rat WT High Dose | 10 mM Tris, 1 mM magnesium chloride (MgCl₂), 150 mM | 259 μL | 6 |
| | sodium chloride (NaCl) and 0.02% poloxamer 188, pH 8.0 | | |
| Rat M3 Vehicle | 10 mM Tris, 1 mM magnesium chloride (MgCl₂), 150 mM | 259 μL | 6 |
| | sodium chloride (NaCl) and 0.02% poloxamer 188, pH 8.0 | | |
| Rat M3 High Dose | 10 mM Tris, 1 mM magnesium chloride (MgCl₂), 150 mM | 259 μL | 6 |
| | sodium chloride (NaCl) and 0.02% poloxamer 188, pH 8.0 | | |
| Rat M3 Medium | 10 mM Tris, 1 mM magnesium chloride (MgCl₂), 150 mM | 259 μL | 6 |
| Dose | sodium chloride (NaCl) and 0.02% poloxamer 188, pH 8.0 | | |
| Rat M3 Low Dose | 10 mM Tris, 1 mM magnesium chloride (MgCl₂), 150 mM | 259 μL | 6 |
| | sodium chloride (NaCl) and 0.02% poloxamer 188, pH 8.0 | | |

The vials will be stored at ≤−60° C. Each vial is thawed just before use at room temperature and active agent will be administered within a maximum of 2 hours post-thawing.

### Study Plan

### Experimental Design:

The full list of rat animal numbers, genotypes, treatment groups and sacrifice timepoints are outlined in the Table 13 below:

TABLE 13

| Genotype | Treatment | T14 | T35 | T180 | Total |
|---|---|---|---|---|---|
| WT | None | 8 (4M/4F) | 8 (4M/4F) | 8 (4M/4F) | 24 |
| (GALT+) | Vehicle | 8 (4M/4F) | 8 (4M/4F) | 8 (4M/4F) | 24 |
| | 1.0E14 vg/kg | 8 (4M/4F) | 8 (4M/4F) | 8 (4M/4F) | 24 |
| M3 | None | 8 (4M/4F) | 8 (4M/4F) | 8 (4M/4F) | 24 |
| (GALT-null) | Vehicle | 8 (4M/4F) | 8 (4M/4F) | 8 (4M/4F) | 24 |
| | 1.0E12 vg/kg | 8 (4M/4F) | 8 (4M/4F) | 8 (4M/4F) | 24 |
| | 1.0E13 vg/kg | 8 (4M/4F) | 8 (4M/4F) | 8 (4M/4F) | 24 |
| | 1.0E14 vg/kg | 8 (4M/4F) | 8 (4M/4F) | 8 (4M/4F) | 24 |

| | | | | |
|---|---|---|---|---|
| Total | 64 | 64 | 64 | 192 |

14 days post-dose = T14;
35 days post-dose = T35;
180 days post-dose = T180;
male = M;
female = F;
wildtype = WT;
pathogenic frame-shift mutation at the active site of the rat Galt gene = M3;
vector genome = vg

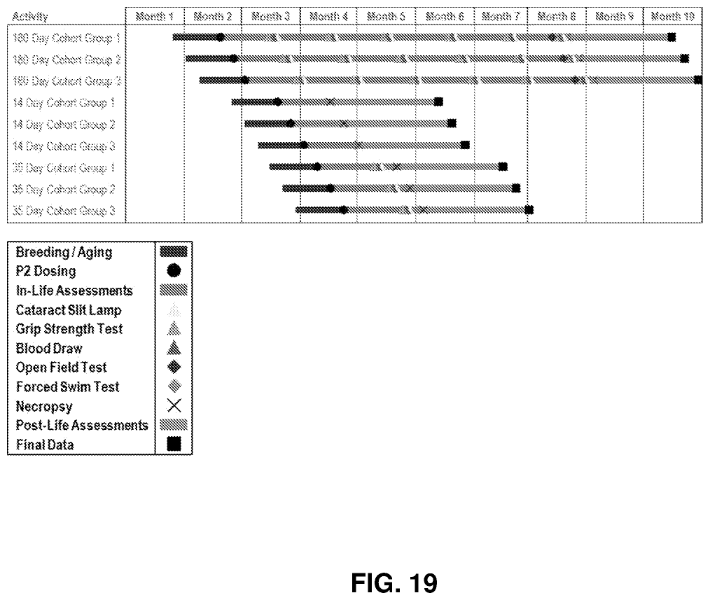

Rats will be dosed at P2 (24-48 hours after estimated time of birth) following weight measurements to calculate the correct dosing volume (7 μL material/gram pup mass). Rats will undergo in-life assessments at monthly intervals and collected at either 14-, 35- or approximately 180-days post-dosing for post-life assessments. The experimental plan for both in-life and post-life assessments are outlined in FIG. 19.

### Breeding:

All rats used in this study will be Sprague Dawley (outbred). GALT-null pups (M3) will derive from crosses of confirmed GALT-null parents; wild-type (WT) pups will derive from crosses of confirmed WT parents. Galt genotypes of all experimental animals will be confirmed by sending tissue samples (tail snip, ear punch, or other tissue sample) to Transnetyx (https://www.transnetyx.com) which performs all rat genotyping. The relevant mutant Galt allele is M3 (Rasmussen et al 2020).

### Enrollment:

Pups must weigh at least 5 grams, must look healthy (pink and “wiggly”), and must be warm and show a visible milk spot (evidence of nursing) to enroll in the study. Pups of both sexes will be included in each comparison group in as close to a 1:1 ratio as possible. Pups from each litter will be assigned randomly to relevant treatment groups, such that each group will include pups of the correct genotype from more than one litter. Comparison group numbers are listed in Table 13 above. Between 24 to <48 hours after birth (P2), pups will be weighed and marked (ink numbers on back+ink on paws for a 2-factor ID), scored visually for presence of a milk spot, and scored visually as male or female. If they meet enrollment criteria, each pup will be assigned to a treatment group, where sex and genotype will be unblinded but treatment groups, except for “untreated,” will remain blinded.

Pups assigned to treatment groups (other than “untreated”) will be administered the appropriate treatment. Following injection and recovery, each pup will be returned to the nest with its littermates and mother. On each subsequent day, each pup will be weighed and remarked with ink. Sex determination will be confirmed at approximately post-natal day 10 once the signs become more evident (e.g., females develop ventral nipple buds; males do not). Just prior to weaning, pups will be ear-punched for identification.

All pups will be weaned, in same-sex pairs or triples, on post-natal day 24 to cages with Lab Diet 5053 chow and water, both available ad libitum.

### Endpoints:

Collection of Cataract Data: While alive, rats will be examined by slit lamp at monthly timepoints post-dosing (approximately 30-day intervals), where both eyes will be dilated and photographed. Immediately post-euthanasia, eyes will also be manually scored using a 4-point scale as defined in Rasmussen et al., JIMD 2020 for the absence or severity of cataract.

Collection of Weight Data: Rats will be weighed daily up until 1 month of age, and weekly (±1 day) thereafter until euthanasia.

Collection of Grip Strength Data: Rats will be assessed for grip strength at monthly timepoints (approximately 30-day intervals) post-dosing, using a custom grip strength testing device. Specifically, each rat is placed on a clean metal wire grid, face forward, supported on low-friction roller bearings (Skelang 1″; zs1818) and sitting on a smooth “runway”. One end of the grid is attached to a Force Meter Spring Scale (QWORK WD3854) so that as the grid is displaced the spring is stretched, registering force on the scale. For each rat, a specific spring scale is used such that the anticipated reading will be near the middle of the range of the scale, e.g., for older rats a 20 Newton scale, while for younger (weaker) rats we use a 5 or ION scale. Once the rat has grasped the grid with all four paws, with the 2 front paws at the proximal edge of the grid, the tester pulls gently on the base of rat's the tail and continues to pull until the rat starts to slip. Grip strength is measured on the Newton spring as the maximum force registered just before the rat starts to lose its grip. At the respective monthly assessment, each rat will be tested once per day for 4 consecutive days.

At 6 months post-dosing a single additional grip strength assessment will be performed with a digital Grip Strength Meter with Rat Pull Bars attached (Columbus Instruments). Once the rat has grasped the Pull Bar the tester pulls gently on the base of rat's tail and continues to pull until the rat starts to slip. Peak grip strength is automatically measured on the digital Grip Strength Meter and recorded for data analysis.

Collection of Open Field Data: Rats will undergo 2 consecutive days of Open Field testing at 6 months (approximately 180 days) post-dosing, using a custom rectangular Open Field arena of approximately 3 ft×4 ft. Rats will be placed into the Open Field arena within the peripheral zone and allowed to freely explore the arena for a period of 10 minutes, which is video recorded from above for data analysis. The rat will be returned into their home cage at the end of the assessment. After each run, any feces are removed, and the arena is thoroughly wiped and cleaned to prevent scents impacting the next Open Field assessment.

Forced Swim Test: Rats will undergo Forced Swim Test assessment at 6 months (approximately 180 days) post-dosing, over a 2-day period. The forced swim test will be performed in cylindrical tanks half-filled with room temperature water, so that the rats while swimming at the surface are not able to reach the bottom or top of the tank. On Day 1 of the Forced Swim Test the rats are placed individually into the water filled tank and undergo a 15-minute acclimatization run, followed by on Day 2 (24 hours later) a second 5-minute testing run. Both Day 1 and Day 2 runs are video recorded and analyzed for activity and immobility time. After each run the wet rats are gently dried before returning to their home cage.

Tail vein blood draws: Up to about 500 μL of blood is collected on a monthly basis from the tail vein of each rat >1-month-old. The blood is collected into a sterile syringe and immediately delivered into a small sample tube pre-loaded with sodium heparin to prevent clotting. Tail vein blood samples are processed as described for post-euthanasia bloods.

Rats will be anesthetized with isoflurane and euthanized with PBS whole body perfusion for tissue harvest at ages 14, 35 or approximately 180 days after treatment (±1 day). Table 14.

TABLE 14
Tissues Collection at euthanasia:

| | | Snap frozen for | | Snap frozen for |
|---|---|---|---|---|
| | Snap frozen for | ddPCR and RNA | Fixed for | Retain Sample |
| | Biochemistry | (not homogenized) | Histology/IHC | (not homogenized) |
| Brain (Left hemisphere) | X | X | — | X |
| Brain (Right hemisphere) | — | — | X | — |
| Eyes | X | — | — | — |
| Liver | X | X | X | X |
| Ovary or Testis | X | — | X^{a} | — |
| Skeletal Muscle | X | X | X | X |
| Blood (plasma) | X | — | — | — |
| Blood (washed RBC) | X | X | — | — |

^{a}= not collected from T14 sacrificed animals

### Collection Method for Vector DNA/RNA/Retain Samples:

All frozen tissue samples for Vector DNA and RNA ddPCR analysis will be shipped in batches on dry ice to Bioagylitix. Retain samples will be at ≤−60° C.

Brain (Left hemisphere)—2 mm thick slice collected from the brain left hemisphere starting at 2/10 of the total distance from the anterior end and divided into three approximately equal pieces each at least 10-25 mg: the dorsal-most piece for Vector DNA, the next piece for RNA, and the ventral-most piece for retain. Each piece will be placed in a pre-labeled round bottom 2 ml microcentrifuge tube then snap-frozen and held at ≤−60° C.

Liver—2 mm thick slice collected from immediately proximal to the slice collected for histology/IHC (see below) from the largest lobe and divided into three pieces each at least 25 mg: one each for Vector DNA, RNA and retain. Each piece will be placed in a pre-labeled round bottom 2 mL microcentrifuge tube, then snap frozen and held at ≤−60° C.

Skeletal Muscle—2 mm thick slice collect from immediately proximal to the slice collected for histology/IHC (see below) from the gastrocnemius muscle in the hind limbs and divided into three pieces each of approximately 10-25 mg: one each for Vector DNA, RNA and retain. Each piece will be placed in a pre-labeled round bottom 2 mL microcentrifuge tube, then snap frozen and held at ≤−60° C.

Blood—Washed Red blood cell pellet should be retained for ddPCR analysis and held at ≤−60° C.

Collection method for Histology/IHC:

Samples will be immersion fixed in 10% neutral buffered formalin for up to 72 hours at room temperature, then transferred to 70% ethanol and stored at room temperature until shipping. Following fixation, tissues will be shipped for histological processing, H&E, and GALT immunohistochemistry. Tissues to be collected include:
Intact brain (right hemisphere)
2-5 mm thick slice of liver from the largest lobe
2-5 mm thick slice of gastrocnemius muscle
Right ovary or testis (from T35 and T180 animals only)

Collection method for biochemical analyses (GALT activity and galactose, galactitol, Gal-1p quantitation):

Tissues for biochemical analyses will be homogenized, aliquoted, snap-frozen, and stored at ≤−60° C. Biochemical analyses will be performed. Samples will include:
Blood (plasma and washed RBC)
Brain (left hemisphere)
Liver (sections not harvested for Vector DNA/RNA/retain or histology/IHC)
Ovary or testis (both combined from younger animals, left ovary or testis from older animals)
Eyes

In the event that a rat previously enrolled into the study has to be sacrificed early or is found dead, blood samples (plasma and washed RBC) will be collected and the necropsy procedure will be performed, if possible. If an animal found dead demonstrates the onset of rigor mortis, then the necropsy procedure should be performed if possible, solely for fixed tissue collection.

Metabolite data from the mouse and rat studies described above are found in Table 15.

TABLE 15
Summary of Metabolite data in Mouse and Rat

| | | | Average Percent Reduction vs | | | |
|---|---|---|---|---|---|---|
| Species | Metabolite | Tissue | Vehicle Control | | | |
| | Timepoint | | P37 | | P93 | |
| | Dose level | | L | H | L | H |
| Mouse | Galactose | Liver | Pending | | 72 | 73 |
| | | Muscle | 34 | 51 | 45 | 52 |
| | | Brain | 55 | 76 | 37 | 56 |
| | | Plasma | Pending | | Pending | |
| | | RBC | 70 | 54 | 46 | 49 |
| | Gal-1P | Liver | Pending | | 72 | 77 |
| | | Muscle | NS | NS | NS | 38 |
| | | Brain | 48 | 59 | NS | 40 |
| | | Plasma | Pending | | Pending | |
| | | RBC | NS | NS | 46 | 70 |
| | Galactitol | Liver | Pending | | 28 | 57 |
| | | Muscle | 58 | 67 | 74 | 78 |
| | | Brain | 27 | 48 | NS | 33 |
| | | Plasma | Pending | | Pending | |
| | | RBC | NS | NS | NS | NS |
| | Timepoint | | P16 | | P37 | |
| | Dose Level | | L | H | L | H |
| Rat | Galactose | Liver | 85 | 99 | 95 | 92 |
| | | Brain | 86 | 96 | 95 | 89 |
| | | Muscle | 99 | 99 | 76 | 80 |
| | | Ovary | 77 | 83 | 30 | NS |
| | | Eye | 70 | 96 | 94 | 94 |
| | | Plasma | 96 | 93 | 97 | 91 |
| | | RBC | 62 | 99 | 97 | 84 |
| | Gal-1P | Liver | 95 | 93 | 95 | 97 |
| | | Brain | NS | 30 | 72 | 89 |
| | | Muscle | 74 | 81 | 66 | 77 |
| | | Ovary | 82 | 67 | NS | NS |
| | | Eye | 37 | 42 | Pending | |
| | | RBC | 52 | 34 | NS | NS |
| | Galactitol | Liver | 81 | 88 | 35 | 32 |
| | | Brain | 85 | 93 | 87 | 79 |
| | | Muscle | 99 | 99 | 95 | 98 |
| | | Ovary | 34 | 76 | 46 | 56 |
| | | Eye | 63 | 79 | 89 | 87 |
| | | Plasma | 89 | 92 | 50 | 46 |
| | | RBC | NS | 90 | 85 | 70 |

Abbreviations: NS = not significant;
L = low dose;
H = high dose
Mouse: P37 = 37 days of age or 4 weeks post-treatment at P2; P93 = 93 days of age or 12 weeks post-treatment at P2
Rat: P16 = 16 days of age or 14 days post-treatment at P9; P37 = 37 days of age or 35 days post-treatment at P9

### REFERENCES

All publications, patent applications, patents, and other references mentioned herein (e.g., sequence database reference numbers) are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of the filing date of this application. When one gene or protein references a plurality of sequence accession numbers, all of the sequence variants are encompassed.
US Patent Application Publication Number US 2003/0138772;
Sambrook et al, Molecular Cloning: A Laboratory Manual, Cold Spring Harbor Press, Cold Spring Harbor, N.Y.;
K. Fisher et al, J. Virol., 70:520-532 (1993);
U.S. Pat. No. 5,478,745;
U.S. Pat. Nos. 6,001,650;
U.S. Pat. No. 6,156,303;
Graham et al. (1973) Virology, 52:456;
Davis et al. (1986) Basic Methods in Molecular Biology, Elsevier;
Chu et al. (1981) Gene 13:197;
Wright F R, et al., Molecular Therapy (2005) 12, 171-178;
U.S. Pat. Nos. 5,543,158;
U.S. Pat. Nos. 5,641,515;
U.S. Pat. No. 5,399,363;
“Remington's Pharmaceutical Sciences” 15th Edition, pages 1035-1038 and 1570-1580;
U.S. Pat. No. 5,741,516;
U.S. Pat. No. 5,567,434;
U.S. Pat. No. 5,552,157;
U.S. Pat. No. 5,565,213;
U.S. Pat. No. 5,738,868;
U.S. Pat. No. 5,795,587;
U.S. Pat. No. 5,656,016;
U.S. Pat. No. 5,779,708;
U.S. Pat. No. 5,797,898;
U.S. Pat. No. 5,770,219;
U.S. Pat. No. 6,083,716;
U.S. Pat. No. 5,871,982;
U.S. Pat. No. 6,251,677;
U.S. Pat. No. 5,783,208; and
U.S. Pat. No. 5,697,899.
Holton (1995) Eur J Pediatr. 154(7 Suppl 2):S77-81.
Berry (2021) GeneReviews®[Internet].
Rubio-Gozalbo et al (2019) Orphanet J Rare Dis, 14(1):86.
Kotb et al (2019) Int J Gen Med. 12:193-205.
Welling et al (2017) J Inherit Metab Dis. 40(2):171-176.
Bosch (2006) J Inherit Metab Dis. 29(4):516-525.
Ozgun et al (2019) Int J of Dev Neurosci. 78:92-97.
Waggoner et al (1990) J Inherit Metab Dis. 13(6):802-818.
Hughes et al (2009) J Pediatr. 154(5):721-726.
Shriberg et al (2011)a J Autism Dev Disord. 41:405-426.
Waisbren et al (2012) J Inherit Metab Dis. 35(2):279-286.
Webb et al (2003) Pediatr Res. 53:396-402.
Potter et al (2008) J Inherit Metab Dis. 31:524-532.
Shriberg et al (2011)b J Speech Lang Hear Res. 54(2):487-519.
Duffy (2005) Motor Speech Disorders: Substrates. Differential Diagnosis, and Management. 2nd Edition. Elsevier Mosby.
American Speech-Language-Hearing Association (2007) Childhood Apraxia of Speech. Technical Report [Internet]
Potter (2011) Potter N L. Voice disorders in children with classic galactosemia. J Inherit Metab Dis. 34(2):377-385
Norbury et al (2016) J Child Psychol Psychiatry. 57(11):1247-1257.
Rasmussen et al (2020) J Inherit Metab Dis. 43(3):518-528.
Tang et al. (2014) Eur J Hum Genet. 22(10):1172-1179.
Pasi et al. (2020) N Engl J Med. 382:29-40.
Balakrishnan et al., (2020) Mol Ther. 28(1):304-312.

We claim:

1. A method for treating galactosemia in a subject in need thereof, the method comprising administering to the subject a therapeutically effective amount of a recombinant AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

2. A method of increasing galactose metabolism in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

3. A method of reducing a disease condition in a subject suffering from galactosemia, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector,
wherein the disease condition comprises jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency.

4. A method of reducing galactose levels in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

5. A method of reducing galactitol levels in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

6. A method of reducing galactose-1-phosphate (Gal-1P) in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

7. A method of increasing GALT protein expression in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

8. A method of increasing GALT enzyme activity in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

9. A method of improving motor and/or neuromuscular coordination comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

10. A method of improving motor strength comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

11. A method of improving spatial learning comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

12. A method of reducing and/or rescuing ovarian failure comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

13. A method of regulating follicle-stimulating hormone, luteinizing hormone and/or anti-Müllerian hormone comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

14. A method of inhibiting cataract formation or promoting cataract resorption in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

15. A method of limiting the severity of cataract formation in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

16. A method of reducing a pre-pubertal growth delay in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

17. A method of improving weight gain in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

18. A method of reducing the severity of a visceral neuromuscular deficit in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

19. A method of reducing the severity of neurological and/or socioemotional deficits in a subject in need thereof, the method comprising administering to the subject a therapeutically effective amount of the AAV virion comprising: 1) an AAV capsid; and 2) a recombinant AAV vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences; and wherein the AAV capsid protein encapsidates the recombinant AAV vector.

20. The method of any one of claims 1 through 19, wherein the hGALT comprises the amino acid sequence of SEQ ID NO.: 1

21. The method of any one of claims 1 through 20, wherein the nucleic acid that encodes GALT comprising a nucleic acid having at least 85% identity to the nucleotide sequence of SEQ ID NO.: 2 or a sequence reverse complementary thereto.

22. The method of any one of claims 1 through 21, wherein the nucleic acid that encodes GALT comprising or consisting of the nucleotide sequence of SEQ ID NO.: 2 or a sequence reverse complementary thereto.

23. The method of any one of claims 1 through 22, wherein the promoter has a nucleotide sequence of SEQ ID NO: 9 or SEQ ID NO: 10, or the reverse complement thereof, and the polyA signal sequence has a nucleotide sequence of SEQ ID NO: 15 or SEQ ID NO: 16, or the reverse complement thereof.

24. The method of any one of claims 1 through 23, which comprises a WPRE element.

25. The method of any one of claims 1 through 24, wherein the ITRs comprise a 5′ AAV2 ITR having a nucleotide sequence of SEQ ID NO: 11 and a 3′ AAV2 ITR having a nucleotide sequence of SEQ ID NO: 12, or the reverse complement thereof.

26. The method of any one of claims 1 through 25, wherein the ITRs comprise a 5′ ITR having a nucleotide sequence of SEQ ID NO: 11 and a modified self-complementary 3′ ITR having a nucleotide sequence of SEQ ID NO: 13, or reverse complement thereof.

27. The method of any one of claims 1 through 26, comprising an expression cassette comprising a nucleic acid that has at least 85% identity to the nucleotide sequence of SEQ ID NO.: 3, SEQ ID NO.: 4, or SEQ ID NO.: 5, or a sequence reverse complementary thereto, and encodes a human GALT.

28. The method of any one of claims 1 through 27, comprising an expression cassette comprising a nucleic acid that has at least 85% identity to the nucleotide sequence of SEQ ID NO.: 19, SEQ ID NO.: 20, or SEQ ID NO.: 21, or a sequence reverse complementary thereto, and encodes a human GALT.

29. The method of any one of claims 1 through 28, comprising an expression cassette comprising a nucleic acid having a nucleotide sequence of SEQ ID NO.: 3, SEQ ID NO.: 4, or SEQ ID NO.: 5, or a sequence reverse complementary thereto.

30. The method of any one of claims 1 through 29, comprising an expression cassette comprising a nucleic acid having a nucleotide sequence of SEQ ID NO.: 19, SEQ ID NO.: 20, or SEQ ID NO.: 21, or a sequence reverse complementary thereto.

31. The method of any one of claims 1 through 30, wherein the recombinant AAV vector is a self-complementary (scAAV).

32. The method of any one of claims 1 through 31, wherein the AAV capsid has an amino acid sequence at least 85% identical to SEQ ID NO: 18 (AAV9).

33. The method of any one of claims 1 through 32, wherein the AAV capsid has an amino acid sequence of SEQ ID NO: 18.

34. The method of any one of claims 1 through 33, said administering comprising intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation.

35. The method of any one of claims 1 through 34, the administering comprising intravenous administration, or intrathecal administration.

36. The method of any one of claims 4-6 or 20-35, wherein galactose levels, galactitol levels and/or Gal-1P levels are reduced in liver, muscle, brain, eye, ovary, red blood cells and/or blood plasma.

37. The method of any one of claims 4-6 or 20 through 36, wherein galactose and galactitol levels are reduced in blood plasma.

38. The method of any one of claims 4-6 or 20 through 37, wherein galactose, galactitol and Gal-1P levels are reduced in red blood cells.

39. The method of any one of claims 7-8 or 20-35, wherein GALT protein expression and/or GALT enzyme activity is increased in liver, muscle, brain, eye, red blood cells and/or blood plasma.

40. The method of any one of claims 7-8, 20-35 or 39, wherein GALT protein expression and/or GALT enzyme activity is increased in brain.

41. The method of any one of claims 7-8, 20-35 or 39-40, wherein GALT protein expression and/or GALT enzyme activity is increased in cortex and/or cerebral tissues.

42. The method of any one of claims 7-8, 20-35 or 39-41, wherein GALT protein expression and/or GALT enzyme activity is increased in neural and/or glial cells.

43. The method of any one of claims 7-8, 20-35 or 39-42, wherein GALT protein expression and/or GALT enzyme activity is increased in Purkinje neurons.

44. The method of any one of claims 14-15 or 20-35, wherein said therapeutically effective amount of the AAV virion is administered prior to the development of cataracts.

45. The method of any of claims 19-35, wherein the neurological and/or socioemotional deficit is locomotor function.

46. The method of claim 45, wherein the locomotor function is tremor and/or ataxia.

47. The method of any one of claims 19-35, wherein the neurological and/or socioemotional deficit is anxiety.

48. The method of any one of claims 19-35, wherein the neurological and/or socioemotional deficit is depression.

49. The method of any one of claims 1-48, wherein the AAV virion is administered prior to onset of puberty.

50. The method of claim 49, wherein the AAV virion is administered to a young pediatric subject or a pediatric subject.

51. The method of any one of claims 1 through 50, further comprising the step of monitoring levels of galactose, GAL-1P and/or galactitol.
